(19) United States
(12) Patent Application Publication
(10) Pub. No.: US 2022/0365070 A1
(43) Pub. Date: Nov. 17, 2022

(54) COMPOSITIONS, SYSTEMS, AND METHODS FOR IDENTIFYING A COMPOUND THAT MODULATES ONE OR MORE CHARACTERISTICS ASSOCIATED WITH A RBM20 CONDENSATE AND/OR A RBM20 POLYPEPTIDE

(71) Applicant: Dewpoint Therapeutics, Inc., Boston, MA (US)

(72) Inventors: Avinash PATEL, Dresden (DE); Mark Andrew MURCKO, Holliston, MA (US); Stephen Paul HALE, Belmont, MA (US); Bruce Aaron BEUTEL, Needham, MA (US); Bozena KESIC, Dresden (DE); Juwina WIJAYA, Boston, MA (US); John C. MANTEIGA, Boston, MA (US); Peter Jeffrey DANDLIKER, Quincy, MA (US); Ann D. KWONG, Pepperell, MA (US)

(21) Appl. No.: 17/761,545

(22) PCT Filed: Sep. 17, 2020
(86) PCT No.: PCT/US2020/051329

§ 371 Date: Mar. 17, 2022

Related U.S. Application Data
(60) Provisional application No. 63/074,985, filed on Sep. 4, 2020.
(60) Provisional application No. 62/902,309, filed on Sep. 18, 2019.

(51) Int. Cl. G01N 33/50

(52) U.S. Cl. CPC G01N 33/5038; G01N 33/5044; G01N 2500/00

(57) ABSTRACT

The present disclosure, in some aspects, is directed to methods for screening and identifying compounds that modulate one or more characteristics associated with a condensate comprising a RBM20 polypeptide and/or the RBM20 polypeptide. In other aspects, the disclosure is directed to systems and composition components thereof, such as cellular models useful for the methods described herein.

### CROSS-REFERENCE TO RELATED APPLICATIONS

This application claims the priority benefit of U.S. Provisional Application No. 62/902,309, filed on Sep. 18, 2019, and U.S. Provisional Application No. 63/074,985, filed on Sep. 4, 2020, the contents of each of which are incorporated herein by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 185992000240SEQLIST.TXT, date recorded: Sep. 16, 2020, size: 11 KB).

### TECHNICAL FIELD

The present disclosure, in some aspects, is directed to methods for screening and identifying compounds that modulate one or more characteristics associated with a condensate comprising a RBM20 polypeptide and/or the RBM20 polypeptide. In other aspects, the disclosure is directed to systems and compositions thereof, such as cellular models useful for the methods described herein.

### BACKGROUND

There are numerous challenges associated with screening and identifying compounds useful for modulating a cellular process, especially when the role of a target biomolecule or a cellular component in a cellular process is not well understood. For example, RBM20 is a protein known to be responsible for the splicing of Titan, a sarcomeric protein that provides structural support and maintains tension during striated muscle stretching. Expression of mutant RBM20 polypeptides is linked with the expression of a pathological Titan isoform which causes Dilated Cardiomyopathy (DCM). Guo et al., Nat Med, 18, 2012. However, it is unclear how RBM20 leads to disease presentation and progression. Thus, development of methods for screening and identifying compounds useful for modulating RBM20-associated cellular processes has been hindered.

All references cited herein, including patent applications and publications, are incorporated by reference in their entirety.

### BRIEF SUMMARY

In some aspects, provided herein is a method of identifying a compound that modulates a characteristic associated with one or more condensates comprising a RBM20 polypeptide (“RBM20 condensates”) and/or the RBM20 polypeptide in the cytoplasm of a cell, the method comprising: (a) admixing the compound and a composition comprising the cell, wherein (i) the cell comprises the one or more RBM20 condensates, and/or (ii) the one or more RBM20 condensates form after the compound contacts the composition; and (b) determining the characteristic associated with the one or more RBM20 condensates and/or the RBM20 polypeptide, wherein a modulation in the characteristic, as compared to a reference, indicates that the compound modulates the characteristic associated with the one or more RBM20 condensates and/or the RBM20 polypeptide.

In some embodiments, the characteristic associated with the one or more RBM20 condensates and/or the RBM20 polypeptide is based on any one or more of the following: (i) location of the one or more RBM20 condensates; (ii) distribution of the one or more RBM20 condensates and/or the RBM20 polypeptide; (iii) number of the one or more RBM20 condensates; (iv) size of the one or more RBM20 condensates; (v) ratio of the amount of one or more RBM20 condensates and a reference condensate; (vi) a functional activity associated with the one or more RBM20 condensates; (vii) composition of the one or more RBM20 condensates; (viii) co-localization of the one or more RBM20 condensates with a biomolecule; (ix) diffusion coefficient of a component of the one or more RBM20 condensates; (x) stability of the one or more RBM20 condensates; (xi) dissolution or reduction in size of the one or more RBM20 condensates; (xii) surface area of the one or more RBM20 condensates; (xiii) sphericity of the one or more RBM20 condensates; (xiv) liquidity of the one or more RBM20 condensates; (xv) solidification of the one or more RBM20 condensates; (xvi) location of the RBM20 polypeptide; (xvii) amount of the RBM20 polypeptide or a precursor thereof; (xviii) condensate partitioning of the RBM20 polypeptide into the one or more RBM20 condensates; (xix) a functional activity associated with the RBM20 polypeptide; (xx) aggregation of the RBM20 polypeptide; (xxi) post-translational modification status of the RBM20 polypeptide; and (xxii) amount of a RBM20 polypeptide degradation product.

In some embodiments, the modulation in the characteristic is based on a decrease in the number of the one or more RBM20 condensates in the cytoplasm of the cell. In some embodiments, the modulation in the characteristic is based on a decrease in the amount of the RBM20 polypeptide or a precursor thereof in the cytoplasm of the cell. In some embodiments, the modulation in the characteristic is based on dissolution or reduction in size of the one or more RBM20 condensates in the cytoplasm of the cell. In some embodiments, the modulation in the characteristic is based on a decrease in the functional activity associated with the one and/or more RBM20 condensates or the RBM20 polypeptide in the cytoplasm of the cell.

In some embodiments, the characteristic associated with the one or more RBM20 condensates and/or the RBM20 polypeptide comprises location of the one or more RBM20 condensates, distribution of the one or more RBM20 condensates and/or the RBM20 polypeptide, number of the one or more RBM20 condensates, size of the one or more RBM20 condensates, and ratio of the amount of one or more RBM20 condensates and a reference condensate. In some embodiments, the characteristic associated with the one or more RBM20 condensates and/or the RBM20 polypeptide comprises composition of the one or more RBM20 condensates, and co-localization of the one or more RBM20 condensates with a biomolecule. In some embodiments, the characteristic associated with the one or more RBM20 condensates and/or the RBM20 polypeptide further comprises the functional activity associated with the one or more RBM20 condensates.

In some embodiments, the characteristic associated with the one or more RBM20 condensates and/or the RBM20 polypeptide comprises stability of the one or more RBM20 condensates, dissolution or reduction in size of the one or more RBM20 condensates, and surface area of the one or more RBM20 condensates.

In some embodiments, the characteristic associated with the one or more RBM20 condensates and/or the RBM20 polypeptide comprises sphericity of the one or more RBM20 condensates, liquidity of the one or more RBM20 condensates, and solidification of the one or more RBM20 condensates.

In some embodiments, the characteristic associated with the one or more RBM20 condensates and/or the RBM20 polypeptide comprises location of the RBM20 polypeptide, and amount of the RBM20 polypeptide or a precursor thereof. In some embodiments, the characteristic associated with the one or more RBM20 condensate and/or the RBM20 polypeptide further comprises post-translational modification status of the RBM20 polypeptide. In some embodiments, the characteristic associated with the one or more RBM20 condensate and/or the RBM20 polypeptide further comprises the functional activity associated with the RBM20 polypeptide.

In some embodiments, the characteristic associated with the one or more RBM20 condensates and/or the RBM20 polypeptide comprises co-localization of the one or more RBM20 condensates with a biomolecule, and diffusion coefficient of a component of the one or more RBM20 condensates.

In some embodiments, the characteristic associated with the one or more RBM20 condensates and/or the RBM20 polypeptide comprises stability of the one or more RBM20 condensates, and dissolution or reduction in size of the one or more RBM20 condensates.

In some embodiments, the characteristic associated with the one or more RBM20 condensates and/or the RBM20 polypeptide comprises surface area of the one or more RBM20 condensates, sphericity of the one or more RBM20 condensates, liquidity of the one or more RBM20 condensates, and solidification of the one or more RBM20 condensates.

In some embodiments, the RBM20 polypeptide is a wild type RBM20 polypeptide. In some embodiments, the RBM20 polypeptide is a mutant RBM20 polypeptide. In some embodiments, the mutant RBM20 polypeptide comprises a mutation in an intrinsically disorder region (IDR). In some embodiments, the mutant RBM20 polypeptide comprises a mutation in an RS-rich region. In some embodiments, the mutant RBM20 polypeptide comprises a mutation in one or more of the following positions: arginine 634, serine 635, arginine 636, serine 637, and proline 638. In some embodiments, the mutant RBM20 polypeptide comprises one or more of the following mutations: R636S, R636C, R636H, R634Q, S637G, P638L, S635A, S635E, and S637E. In some embodiments, the mutant RBM20 polypeptide comprises one or more of the following mutations: E913K, R716Q, and V535L.

In some embodiments, the RBM20 polypeptide is heterologously expressed in the cell. In some embodiments, the RBM20 polypeptide is homologously expressed in the cell.

In some embodiments, the cell is a model of a cardiac cell type. In some embodiments, the cell is a cardiomyocyte. In some embodiments, the cell is a Rat H9C2 cell. In some embodiments, the cell is a human AC-16 cell, a patient-derived cardiomyocyte, a human induced pluripotent stem cell differentiated to a cardiomyocyte, or a stem cell differentiated to a cardiomyocyte. In some embodiments, the cell is selected from the group consisting of: a HeLa cell, a U2OS cell, a human embryonic kidney cell, a human induced pluripotent stem cell, and a stem cell. In some embodiments, the cell is homozygous for alleles encoding the RBM20 polypeptide. In some embodiments, the cell is heterozygous for alleles encoding the RBM20 polypeptide.

In some embodiments, the reference comprises an aliquot of the composition comprising the cell admixed with a control agent.

In some embodiments, the method described herein further comprises imaging at least a portion of the composition or the cell.

In some embodiments, the method described herein further comprises determining one or more cellular features of the cell.

In some embodiments, the method described herein further comprises contacting at least a portion of the composition or the cell with a fixative.

In some embodiments, the method described herein further comprises contacting at least a portion of the composition or the cell with a stain.

In some embodiments, the method described herein further comprises assessing the identified compound using a second cell-based assay.

In some embodiments, the method described herein further comprises assessing the identified compound using an in vitro assay.

In another aspect, provided herein is a method of identifying a compound that reduces the size and/or number of condensates comprising a RBM20 polypeptide (“RBM20 condensates”) in the cytoplasm of a cell, the method comprising: (a) determining the size and/or number of the RBM20 condensates in at least a portion of the cytoplasm of the cell subjected to the compound; and (b) comparing the size and/or number of the RBM20 condensates with a reference, thereby identifying the compound that reduces the size and/or number of the RBM20 condensates in the cytoplasm of the cell.

In some embodiments, the compound reduces the number of RBM20 condensates. In some embodiments, the compound reduces the size of RBM20 condensates.

In another aspect, provided herein is a method of identifying a compound that prevents formation or growth of one or more condensates comprising a RBM20 polypeptide (“RBM20 condensates”) in the cytoplasm of a cell, the method comprising: (a) combining the compound and a composition comprising the cell, wherein (i) the cell comprises the one or more RBM20 condensates, and/or (ii) the one or more RBM20 condensates form after the compound contacts the composition; and (b) obtaining a first measurement of the size and/or number of the one or more RBM20 condensates in at least a portion of the cytoplasm of the cell; and (c) comparing the first measurement to a reference, thereby identifying a compound that prevents formation or growth of the one or more RBM20 condensates in the cytoplasm of the cell.

In some embodiments, the reference comprises an aliquot of the composition comprising the cell admixed with a control agent. In some embodiments, the reference is a second measurement of the size and/or number of the one or more RBM20 condensates in at least a portion of the cytoplasm of the cell, and wherein the second measurement is taken at a different time than the first measurement.

In some embodiments, the method described herein further comprises subjecting the cell to a condition that promotes formation of the one or more RBM20 condensates.

In another aspect, provided herein is a method of identifying a compound that decreases the amount of a RBM20 polypeptide in the cytoplasm of a cell, the method comprising: (a) combining the compound and a composition comprising the cell; and (b) obtaining a first measurement of the amount of the RBM20 polypeptide in at least a portion of the cytoplasm of the cell; and (c) comparing the first measurement to a reference, thereby identifying a compound that decreases the amount of the RBM20 polypeptide in the cytoplasm of the cell.

In some embodiments, the reference comprises an aliquot of the composition comprising the cell admixed with a control agent.

In some embodiments, the reference is a second measurement of the amount of the RBM20 polypeptide in at least a portion of the cytoplasm of the cell, and wherein the second measurement is taken at a different time than the first measurement.

In another aspect, provided herein is a method of identifying a compound that modulates a characteristic associated with one or more condensates comprising a RBM20 polypeptide (“RBM20 condensates”), the method comprising: (a) admixing the compound and a solution comprising the one or more RBM20 condensates and an extra-condensate solution; and (b) determining the characteristic associated with the one or more RBM20 condensates, wherein a modulation in the characteristic, as compared to a reference, indicates that the compound modulates the characteristic associated with the one or more RBM20 condensates.

In another aspect, provided herein is a method of identifying a compound that modulates a characteristic associated with one or more condensates comprising a RBM20 polypeptide (“RBM20 condensates”) and/or the RBM20 polypeptide, the method comprising: (a) combining an agent and a solution comprising the RBM20 polypeptide in the presence of the compound, wherein the agent is capable of causing the formation of the one or more RBM20 condensates and the one or more RBM20 condensates form after the agent contacts the solution; and (b) determining the characteristic associated with the one or more RBM20 condensates and/or the RBM20 polypeptide, wherein a modulation in the characteristic, as compared to a reference, indicates that the compound modulates the characteristic associated with the one or more RBM20 condensates and/or the RBM20 polypeptide.

In some embodiments, the characteristic associated with the one or more RBM20 condensates and/or the RBM20 polypeptide is based on any one or more of the following: (i) number of the one or more RBM20 condensates; (ii) composition of the one or more RBM20 condensates; (iii) size of the one or more RBM20 condensates; (iv) stability of the one or more RBM20 condensates; (v) dissolution or reduction in size of the one or more RBM20 condensates; (vi) surface area of the one or more RBM20 condensates; (vii) sphericity of the one or more RBM20 condensates; (viii) liquidity of the one or more RBM20 condensates; (ix) solidification of the one or more RBM20 condensates; (x) amount of the RBM20 polypeptide not in the one or more RBM20 condensates; (xi) partitioning of the RBM20 polypeptide into the one or more RBM20 condensates; and (xii) aggregation of the RBM20 polypeptide.

In another aspect, provided herein is a method of identifying a compound useful for treating a RBM20-associated disease, the method comprising identifying a compound according to any one of the methods described herein. In some embodiments, the RBM20-associated disease is a cardiomyopathy. In some embodiments, the cardiomyopathy is dilated cardiomyopathy.

In another aspect, provided herein is a method of identifying a compound that modulates the partitioning of a biomolecule for a condensate comprising a RBM20 polypeptide (“RBM20 condensate”), the method comprising: (a) admixing the compound and a composition comprising a cell, wherein (i) the cell comprises the RBM20 condensate, and/or (ii) the RBM20 condensate forms in the cell after the compound contacts the composition; and (b) determining the partitioning of the biomolecule for the RBM20 condensates. In some embodiments, the biomolecule is a non-RBM20 polypeptide. In some embodiments, the biomolecule is a wild type RBM20 polypeptide. In some embodiments, the RBM20 condensate comprising the RBM20 polypeptide comprises a mutant RBM20 polypeptide.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### BRIEF DESCRIPTION OF THE DRAWINGS

FIGS. 1A and 1B show fluorescent images of HeLa cells transiently transfected with a wild type RBM20 polypeptide (FIG. 1A) or a R636S mutant RBM20 polypeptide (FIG. 1B).

FIGS. 2A and 2B show fluorescent images of U2OS cells transiently transfected with a wild type RBM20 polypeptide (FIG. 2A) or a R636S mutant RBM20 polypeptide (FIG. 2B).

FIGS. 3A-3D show fluorescent images of U2OS cells transiently transfected with a wild type RBM20 polypeptide (FIG. 3A), a R636S mutant RBM20 polypeptide (FIG. 3B), a R636C mutant RBM20 polypeptide (FIG. 3C), or a R636H mutant RBM20 polypeptide (FIG. 3D).

FIGS. 4A-4H show fluorescent images of H9C2 cells transiently transfected with a wild type RBM20 polypeptide (FIG. 4A), a R636S mutant RBM20 polypeptide (FIG. 4B), a R636C mutant RBM20 polypeptide (FIG. 4C), a R636H mutant RBM20 polypeptide (FIG. 4D), a R634Q mutant RBM20 polypeptide (FIG. 4E), a S635A mutant RBM20 polypeptide (FIG. 4F), a S637G mutant RBM20 polypeptide (FIG. 4G), or a P638L mutant RBM20 polypeptide (FIG. 4H).

FIGS. 5A and 5B show fluorescent images of U2OS cells transiently transfected with a wild type RBM20 polypeptide.

FIGS. 6A-6D show fluorescent images of H9C2 cells transiently transfected with a wild type RBM20 polypeptide and treated with a control (DMSO) (FIG. 6A), lipoamide (30 μM) (FIG. 6B), mitoxantrone (20 μM) (FIG. 6C), or JQ1 (10 μM) (FIG. 6D).

FIG. 7A is a schematic illustrating select regions of the RBM20 polypeptide. FIG. 7B shows the results of an analysis of the RBM20 polypeptide sequence for ordered and disordered regions.

FIGS. 8A and 8B show fluorescent images of H9C2 cells transiently transfected to express a wild type RBM20 polypeptide.

FIG. 9 shows fluorescent images of H9C2 cells transiently transfected to express a R636S mutant RBM20 polypeptide with a C-terminus NLS fusion (upper panels) or without C-terminus NLS fusion (bottom panels).

FIG. 10 shows fluorescent images of H9C2 cells transiently transfected to express a phospho-mimetic mutant RBM20 polypeptide (R636S, S635E, S637E) linked to a Dendra2 label, or a mutant R636S RBM20 polypeptide linked to a Dendra2 label.

FIG. 11 shows fluorescent images of H9C2 cells transiently transfected to express various RBM20 polypeptide truncation forms.

FIGS. 12A-12D show fluorescent images and analyses of H9C2 cells transiently transfected to express (i) both a mutant R636S RBM20 polypeptide and a wild type RBM20 polypeptide; (ii) both a mutant R636S RBM20 polypeptide and a NEC polypeptide; (iii) a mutant R636S RBM20 polypeptide; or (iv) a wild type RBM20 polypeptide.

FIGS. 13A-13D show fluorescent images of H9C2 cells transiently transfected to express a wild type RBM20 polypeptide (FIG. 13A), an E913K mutant RBM20 polypeptide (FIG. 13B), a R716Q mutant RBM20 polypeptide (FIG. 13C), and a V535L mutant RBM20 polypeptide (FIG. 13D), each linked to a Dendra2 label.

FIG. 14 shows fluorescent images of H9C2 cells transiently transfected to express a wild type RBM20 polypeptide (upper panels) or a mutant RBM20 polypeptide (bottom panels). Cells were co-stained for DAPI (to show the nucleus) and DDX3X protein (using an IF technique).

FIG. 15 shows fluorescent images of H9C2 cells transiently transfected to express a wild type RBM20 polypeptide (upper panels) or a mutant RBM20 polypeptide (bottom panels). Cells were co-stained for DAPI (to show the nucleus) and PSPC1 protein (using an IF technique).

FIG. 16 shows fluorescent images of H9C2 cells transiently transfected to express a Dendra-labeled wild type RBM20 polypeptide or a Dendra-labeled R636S mutant RBM20 polypeptide. Cells were stained for various nuclear proteins PTBP1, SRRM1, U2AF65, and SC35.

FIG. 17 shows fluorescent images of H9C2 cells transiently transfected to express a mCherry-labeled wild type RBM20 polypeptide or a mCherry-labeled R636S mutant RBM20 polypeptide, in combination with a GFP-labeled DDX3X. Cells were co-stained for DAPI and stress granule marker G3BP1 protein.

FIG. 18 shows fluorescent and DIC images of H9C2 cells induced to express either a wild type RBM20 polypeptide linked to a Dendra2 label, or a R636S mutant RBM20 polypeptide linked to a Dendra2 label.

FIGS. 19A-19D show cell proliferation curves of H9C2 cells induced to express either a wild type RBM20 polypeptide or a R636S mutant RBM20 polypeptide, stained with Incucyte® NucLight Rapid Red Reagent. No addition of doxycycline served as controls.

FIGS. 20A-20E show apoptotic analyses H29C cells expressing either a wild type RBM20 polypeptide or a R636S mutant RBM20 polypeptide, with or without Staurosporine (STS) stress. The extent of apoptosis was indicated by luminescence (RLU) signal.

FIG. 21 shows fluorescent and DIC images of H29C cells (not treated with STS stress) induced to express either wild type or R636S mutant RBM20 polypeptides.

FIG. 22 shows fluorescent images of H9C2 cells induced to express R636S mutant RBM20 polypeptides labeled with Dendra2 that formed cytoplasmic condensates, and co-stained with DAPI and stress granule protein elF3e.

FIG. 23 shows fluorescent images of H9C2 cells transiently transfected to express a R636S mutant RBM20 polypeptide. DMSO, lithocholic acid, and Quinacrine 2HCl were added to cells to monitor cytoplasmic R636S mutant RBM20 condensate behavior. Non-transfected H9C2 cells served as control.

FIGS. 24A-24E show fluorescent images of H9C2 cells transiently transfected to express a R636S mutant RBM20 polypeptide. Compounds Triptolide (PG490) (FIG. 24A), BIO (FIG. 24B), Uprosertib (GSK2141795) (FIG. 24C), anisomycin (FIG. 24D), and WS6 (FIG. 24E) were all added to monitor cytoplasmic R636S mutant RBM20 condensate behavior.

### DETAILED DESCRIPTION

The present disclosure provides compositions, systems, and methods for identifying a compound that modulates one or more characteristics associated with one or more condensates comprising a RBM20 polypeptide (“RBM20 condensates”) and/or an RBM20 polypeptide. The disclosure described herein is based, at least in part, on the unexpected findings and the inventors' unique insights regarding disease mechanisms associated with dysfunctional and/or displaced RBM20 polypeptides and aberrant RBM20 condensates, and compositions, systems, and methods for screening and identifying compounds useful for modulating cellular pathways associated RBM20 condensantes and/or RBM20 polypeptides. Under normal physiological conditions, wild type RBM20 is produced in the cytoplasm and then transported to the nucleus where it performs biological functions, such as involved with the splicing of RNAs encoding proteins including Titan. As described herein, expression of certain mutant RBM20 polypeptides and/or overexpression of a RBM20 polypeptide (including a wild type RBM20 polypeptide) results in the presence and maintenance of the RBM20 polypeptide and RBM20 condensates in the cytoplasm of a cell. Without being bound by theory, the inventors believe that the presence of RBM20 polypeptides and/or RBM20 condensates in the cytoplasm leads to aberrant molecular processes and disease development and presentation, such as dilated cardiomyopathy (DCM). For example, cytoplasmic RBM20 condensates, such as those containing a mutant RBM20 polypeptide, may serve as a sink for certain biomolecules including proteins, such as wild type RBM20 polypeptides, and nucleic acids. This in turn may lead to a gain of toxic function impacting cell viability, cell cytotoxicity, and cell proliferation. The approaches described herein are useful for identifying and understanding characteristics associated with RMB20-associated disease mechanisms and such tools and knowledge further enable compositions, systems, and methods useful for screening for compounds having a desired impact on the one or more characteristics of a RBM20 condensate and/or a RBM20 polypeptide. Such compositions, systems, and methods may also be integrated in a drug discovery platform to enhance candidate identification and lead optimization. The identification of compounds that modulate a characteristic associated with one or more RBM20 condensates and/or the RBM20 polypeptide in the cytoplasm of a cell may lead to the identification of compounds useful for the treatment or prevention of aberrant molecular processes, diseases, and/or symptoms associated with the presence of cytoplasmic RBM20 condensates and/or a RBM20 polypeptide.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### I. DEFINITIONS

For purposes of interpreting the specification, the following definitions will apply and whenever appropriate, terms used in the singular will also include the plural and vice versa. In the event that any definition set forth below conflicts with any document incorporated herein by reference, the definition set forth shall control.

As used herein, “condensate” means a non-membrane-encapsulated compartment formed by phase separation of one or more of proteins and/or other macromolecules (including all stages of phase separation).

The terms “polypeptide” and “protein,” as used herein, may be used interchangeably to refer to a polymer comprising amino acid residues, and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

### II. METHODS OF IDENTIFYING A COMPOUND

The present disclosure provides, in some aspects, methods of identifying a compound (or a portion thereof) that modulates a characteristic associated with one or more condensates comprising a RBM20 polypeptide (“RBM20 condensates”) and/or a RBM20 polypeptide. In some embodiments, the one or more characteristics are associated with a cellular mechanism associated with RBM20, and modulation of the one or more characteristics results in a desired modulation of the cellular mechanism. Certain methods described herein focus on identifying/assessing/modulating the one or more characteristics associated with RBM20 condensates and/or RBM20 polypeptides in the cytoplasm, and such methods can also be used for identifying/assessing/modulating the one or more characteristics associated with RBM20 condensates and/or RBM20 polypeptides in any part of a cellular system, such as in the cytoplasm, nucleus, or other cellular organelles, or a biochemical system. In some embodiments, the RBM20 condensate and/or the RBM20 polypeptide (e.g., a wild type RBM20 polypeptide) is in the cell nucleus. In some embodiments, the RBM20 condensate and/or the RBM20 polypeptide (e.g., a mutant RBM20 polypeptide) is in the cytoplasm. Certain methods described herein can also be used for identifying/assessing/modulating one or more characteristics associated with non-RBM20 condensates and/or non-RBM20 polypeptides, for example, modulating a biomolecule that is not RBM20 by releasing it from the cytoplasmic RBM20 condensates back to its normal location (e.g., the location, such as a non-RBM20 condensate, where the biomolecule used to reside before getting sequestered in the cytoplasmic RBM20 condensates). Therefore, certain compounds (or a portion thereof) described herein modulate one or more characteristics associated with non-RBM20 condensates and/or non-RBM20 polypeptides, which in turn modulate the one or more characteristics associated with RBM20 condensates and/or RBM20 polypeptides described herein.

In some embodiments, the methods described herein may be used to identify, assess, screen, and/or design a compound that modulates the partitioning of a molecule (such as a biomolecule, including a protein or nucleic acid) in a RBM20 condensate. For example, in some embodiments, the RBM20 condensate sequesters a molecule (such as a biomolecule including wild type RBM20 or a non-RBM20 polypeptide), and the methods described herein may be used to identify, assess, screen, and/or design a compound that drives the molecule out of the RBM20 condensate. In some embodiments, the methods described herein may be used to identify, assess, screen, and/or design a compound that drives a wild type RBM20 polypeptide out of the RBM20 condensate. In some embodiments, the methods described herein may be used to identify, assess, screen, and/or design a compound that drives a non-RBM20 polypeptide out of the RBM20 condensate. In some embodiments, the molecule (such as a biomolecule including wild type RBM20 or a non-RBM20 polypeptide) functions normally (such as has a normal biological function and/or activity) once out of the RBM20 condensate.

In some embodiments, the methods described herein may be used to identify, assess, screen, and/or design a compound that drives the molecule into the RBM20 condensate. In some embodiments, the methods described herein may be used to identify, assess, screen, and/or design a compound that drives a non-RBM20 polypeptide into the RBM20 condensate.

In some embodiments, described herein is a method of identifying a compound (or a portion thereof) that modulates a characteristic associated with one or more condensates comprising a RBM20 polypeptide (“RBM20 condensates”) in the cytoplasm of a cell, the method comprising: (a) admixing the compound and a composition comprising the cell, wherein (i) the cell comprises the one or more RBM20 condensates, and/or (ii) the one or more RBM20 condensates form after the compound contacts the composition; and (b) determining the characteristic associated with the one or more RBM20 condensates, wherein a modulation in the characteristic, as compared to a reference, indicates that the compound modulates the characteristic associated with the one or more RBM20 condensates.

In some embodiments, described herein is a method of identifying a compound (or a portion thereof) that modulates a characteristic associated with a RBM20 polypeptide in the cytoplasm of a cell, the method comprising: (a) admixing the compound and a composition comprising the cell; and (b) determining the characteristic associated with the RBM20 polypeptide, wherein a modulation in the characteristic, as compared to a reference, indicates that the compound modulates the characteristic associated with the RBM20 polypeptide.

In some embodiments, described herein is a method of identifying a compound (or a portion thereof) that modulates one or more characteristics associated with one or more condensates comprising a RBM20 polypeptide (“RBM20 condensates”) and the RBM20 polypeptide in the cytoplasm of a cell, the method comprising: (a) admixing the compound and a composition comprising the cell, wherein (i) the cell comprises the one or more RBM20 condensates, and/or (ii) the one or more RBM20 condensates form after the compound contacts the composition; and (b) determining the one or more characteristics associated with the one or more RBM20 condensates and the RBM20 polypeptide, wherein a modulation in the characteristic, as compared to a reference, indicates that the compound modulates the one or more characteristics associated with the one or more RBM20 condensates and the RBM20 polypeptide.

In some embodiments, described herein is a method of identifying a compound (or a portion thereof) that modulates a characteristic associated with one or more condensates comprising a RBM20 polypeptide (“RBM20 condensates”) in the cytoplasm of a cell, the method comprising determining the characteristic associated with the one or more RBM20 condensates in a composition comprising the cell and the compound or a derivative thereof, wherein a modulation in the characteristic, as compared to a reference, indicates that the compound modulates the characteristic associated with the one or more RBM20 condensates, and wherein (i) the cell comprises the one or more RBM20 condensates, and/or (ii) the one or more RBM20 condensates form after the compound contacts the composition.

In some embodiments, described herein is a method of identifying a compound (or a portion thereof) that modulates a characteristic associated with a RBM20 polypeptide in the cytoplasm (or nucleus) of a cell, the method comprising determining the characteristic associated with the RBM20 polypeptide in a composition comprising the cell and the compound or a derivative thereof, wherein a modulation in the characteristic, as compared to a reference, indicates that the compound modulates the characteristic associated with the RBM20 polypeptide.

In some embodiments, described herein is a method of identifying a compound (or a portion thereof) that modulates one or more characteristics associated with one or more condensates comprising a RBM20 polypeptide (“RBM20 condensates”) and the RBM20 polypeptide in the cytoplasm of a cell, the method comprising determining the one or more characteristics associated with the one or more RBM20 condensates and the RBM20 polypeptide in a composition comprising the cell and the compound or a derivative thereof, wherein a modulation in the characteristic, as compared to a reference, indicates that the compound modulates the one or more characteristics associated with the one or more RBM20 condensates and the RBM20 polypeptide, and wherein (i) the cell comprises the one or more RBM20 condensates, and/or (ii) the one or more RBM20 condensates form after the compound contacts the composition.

In some embodiments, described herein is a method of identifying a compound (or a portion thereof) that modulates a characteristic associated with one or more condensates comprising a RBM20 polypeptide (“RBM20 condensates”) in a cell system, the method comprising: (a) admixing the compound and a composition comprising a cell, wherein (i) the cell comprises the one or more RBM20 condensates, and/or (ii) the one or more RBM20 condensates form after the compound contacts the composition; and (b) determining the characteristic associated with the one or more RBM20 condensates, wherein a modulation in the characteristic, as compared to a reference, indicates that the compound modulates the characteristic associated with the one or more RBM20 condensates.

In some embodiments, described herein is a method of identifying a compound (or a portion thereof) that modulates a characteristic associated with a RBM20 polypeptide in a cell system, the method comprising: (a) admixing the compound and a composition comprising a cell; and (b) determining the characteristic associated with the RBM20 polypeptide, wherein a modulation in the characteristic, as compared to a reference, indicates that the compound modulates the characteristic associated with the RBM20 polypeptide.

In some embodiments, described herein is a method of identifying a compound (or a portion thereof) that modulates one or more characteristics associated with one or more condensates comprising a RBM20 polypeptide (“RBM20 condensates”) and the RBM20 polypeptide in a cell system, the method comprising: (a) admixing the compound and a composition comprising a cell, wherein (i) the cell comprises the one or more RBM20 condensates, and/or (ii) the one or more RBM20 condensates form after the compound contacts the composition; and (b) determining the one or more characteristics associated with the one or more RBM20 condensates and the RBM20 polypeptide, wherein a modulation in the characteristic, as compared to a reference, indicates that the compound modulates the one or more characteristics associated with the one or more RBM20 condensates and the RBM20 polypeptide.

In some embodiments, described herein is a method of identifying a compound (or a portion thereof) that modulates a characteristic associated with one or more condensates comprising a RBM20 polypeptide (“RBM20 condensates”) in a cell system, the method comprising determining the characteristic associated with the one or more RBM20 condensates in a composition comprising a cell and the compound or a derivative thereof, wherein a modulation in the characteristic, as compared to a reference, indicates that the compound modulates the characteristic associated with the one or more RBM20 condensates, and wherein (i) the cell comprises the one or more RBM20 condensates, and/or (ii) the one or more RBM20 condensates form after the compound contacts the composition.

In some embodiments, described herein is a method of identifying a compound (or a portion thereof) that modulates a characteristic associated with a RBM20 polypeptide in a cell system, the method comprising determining the characteristic associated with the RBM20 polypeptide in a composition comprising a cell and the compound or a derivative thereof, wherein a modulation in the characteristic, as compared to a reference, indicates that the compound modulates the characteristic associated with the RBM20 polypeptide.

In some embodiments, described herein is a method of identifying a compound (or a portion thereof) that modulates one or more characteristics associated with one or more condensates comprising a RBM20 polypeptide (“RBM20 condensates”) and the RBM20 polypeptide in a cell system, the method comprising determining the one or more characteristics associated with the one or more RBM20 condensates and the RBM20 polypeptide in a composition comprising a cell and the compound or a derivative thereof, wherein a modulation in the characteristic, as compared to a reference, indicates that the compound modulates the one or more characteristics associated with the one or more RBM20 condensates and the RBM20 polypeptide, and wherein (i) the cell comprises the one or more RBM20 condensates, and/or (ii) the one or more RBM20 condensates form after the compound contacts the composition. In some embodiments, described herein is a method of identifying a compound (or a portion thereof) that modulates a characteristic associated with one or more condensates comprising a RBM20 polypeptide (“RBM20 condensates”), the method comprising: (a) admixing the compound and a solution comprising the one or more RBM20 condensates and an extra-condensate solution; and (b) determining the characteristic associated with the one or more RBM20 condensates, wherein a modulation in the characteristic, as compared to a reference, indicates that the compound modulates the characteristic associated with the one or more RBM20 condensates.

In some embodiments, described herein is a method of identifying a compound (or a portion thereof) that modulates a characteristic associated with one or more condensates comprising a RBM20 polypeptide (“RBM20 condensates”) and/or the RBM20 polypeptide, the method comprising: (a) combining an agent and a solution comprising the RBM20 polypeptide in the presence of the compound, wherein the agent is capable of causing the formation of the one or more RBM20 condensates and the one or more RBM20 condensates form after the agent contacts the solution; and (b) determining the characteristic associated with the one or more RBM20 condensates and/or the RBM20 polypeptide, wherein a modulation in the characteristic, as compared to a reference, indicates that the compound modulates the characteristic associated with the one or more RBM20 condensates and/or the RBM20 polypeptide.

In some embodiments, the characteristic associated with the one or more RBM20 condensates is based on any one or more of the following: (i) location of the one or more RBM20 condensates; (ii) distribution of the one or more RBM20 condensates and/or the RBM20 polypeptide; (iii) number of the one or more RBM20 condensates; (iv) size of the one or more RBM20 condensates; (v) ratio of the amount of one or more RBM20 condensates and a reference condensate; (vi) a functional activity associated with the one or more RBM20 condensates; (vii) composition of the one or more RBM20 condensates; (viii) co-localization of the one or more RBM20 condensates with a biomolecule; (ix) diffusion coefficient of a component of the one or more RBM20 condensates; (x) stability of the one or more RBM20 condensates; (xi) dissolution or reduction in size of the one or more RBM20 condensates; (xii) surface area of the one or more RBM20 condensates; (xiii) sphericity of the one or more RBM20 condensates; (xiv) liquidity of the one or more RBM20 condensates; and (xv) solidification of the one or more RBM20 condensates. In some embodiments, the characteristic associated with the RBM20 polypeptide is based on any one or more of the following: (i) location of the RBM20 polypeptide; (ii) amount of the RBM20 polypeptide or a precursor thereof; (iii) condensate partitioning of the RBM20 polypeptide into the one or more RBM20 condensates; (iv) a functional activity associated with the RBM20 polypeptide; (v) aggregation of the RBM20 polypeptide; (vi) post-translational modification status of the RBM20 polypeptide; and (vii) amount of a RBM20 polypeptide degradation product.

The methods described herein may take many forms (e.g., cell-based methods or biochemical methods, such as an in vitro method), in low-throughput or high-throughput, and the components used therein (e.g., a cell or a RBM20 polypeptide) can be used in numerous manners, including methods other than described herein. For example, in some embodiments, the methods described herein may be used, e.g., to identify a compound that modulates any one or more of: the nuclear translocation of a RBM20 polypeptide, a cytoplasmic RBM20 polypeptide, such as modulation in location or function, or a cytoplasmic RBM20 condensate, such as modulation in location or function. The description of certain embodiments of the methods, and components thereof, provided herein is not intended to limit the scope of the disclosure, and it will be readily understood by those skilled in the art that changes in the form and details of the implementations described herein may be made without departing from the scope of this disclosure.

### A. Cell-Based Methods

In some aspects, the method described herein is a cell-based method. One of ordinary skill in the art will readily recognize that cellular processes, including the state of a condensate and components thereof, are dynamic. The methods described herein thus encompass contacting a cell with a compound at any point in the life cycle of a RBM20 polypeptide and/or a RBM20 condensates. For example, the methods encompass contacting a cell with a compound when a RBM20 polypeptide is in any location of the cell, in any quantity, or has any post-translation modification status, such as the presence, absence, or level of a phosphorylated residue. In some aspects, the methods may also encompass, e.g., contacting a cell with a compound when a RBM20 condensates is in any location of the cell, is present in any quantity, including being absent, is undergoing a morphological change, such as a change in size or liquidity, or is changing in composition. In some embodiments, the method comprises admixing the compound and a composition comprising the cell, wherein (i) the cell comprises the one or more RBM20 condensates, and/or (ii) the one or more RBM20 condensates form after the compound contacts the composition. In some embodiments, the method comprises admixing the compound and a composition comprising the cell, wherein the cell comprises the one or more RBM20 condensates. In some embodiments, the method comprises admixing the compound and a composition comprising the cell, wherein the cell the one or more RBM20 condensates form after the compound contacts the composition. In some embodiments, the method comprises forming the one or more RBM20 condensates prior to admixing or contacting the compound and the composition comprising the cell. In some embodiments, the method comprises forming the one or more RBM20 condensates after admixing or contacting the compound and the composition comprising the cell. In some embodiments, admixing the compound and the composition comprising the cell leads to the formation of one or more RBM20 condensates. In some embodiments of the methods described herein, RBM20 condensate formation may be triggered, e.g., by inducing expression of a polypeptide, such as a RBM20 polypeptide or a condensate scaffold protein, or by adding a crowding agent. In some embodiments of the methods described herein, two or more, such as any of 2, 3, 4, or 5, compounds are admixed with the composition comprising the cell. In some embodiments of the methods described herein, the composition comprising the cell is contacted with the compound more than once, e.g., more than one aliquot of the compound is admixed with the composition comprising the cell at more than one point in time.

i. Characteristics Associated with One or More RBM20 Condensates and/or the RBM20 Polypeptide in a Cell-Based Method

In some aspects, described herein are methods of identifying a compound (or a portion thereof) that modulates a characteristic (including one or more, such as 1, 2, 3, 4, or 5 characteristics), associated with one or more condensates comprising a RBM20 polypeptide (“RBM20 condensates”) and/or the RBM20 polypeptide in the cytoplasm (or another component) of a cell. In some embodiments, the method identifies a compound that modulates a characteristic associated with one or more RBM20 condensates. In some embodiments, the method identifies a compound that modulates a characteristic associated with a RBM20 polypeptide. In some embodiments, the characteristic associated with the one or more RBM20 condensates and/or RBM20 polypeptides is based on any one or more of the following: (i) location of the one or more RBM20 condensates; (ii) distribution of the one or more RBM20 condensates and/or the RBM20 polypeptide; (iii) number of the one or more RBM20 condensates; (iv) size of the one or more RBM20 condensates; (v) ratio of the number of one or more RBM20 condensates and a reference condensate; (vi) a functional activity associated with the one or more RBM20 condensates; (vii) composition of the one or more RBM20 condensates; (viii) co-localization of the one or more RBM20 condensates with a biomolecule; (ix) diffusion coefficient of a component of the one or more RBM20 condensates; (x) stability of the one or more RBM20 condensates; (xi) dissolution or reduction in size of the one or more RBM20 condensates; (xii) surface area of the one or more RBM20 condensates; (xiii) sphericity of the one or more RBM20 condensates; (xiv) liquidity of the one or more RBM20 condensates; (xv) solidification of the one or more RBM20 condensates; (xvi) location of the RBM20 polypeptide; (xvii) amount of the RBM20 polypeptide or a precursor thereof; (xviii) condensate partitioning of the RBM20 polypeptide into the one or more RBM20 condensates; (xix) a functional activity associated with the RBM20 polypeptide; (xx) aggregation of the RBM20 polypeptide; (xxi) post-translational modification status of the RBM20 polypeptide; and (xxii) amount of a RBM20 polypeptide degradation product.

In some embodiments, the location of the one or more RBM20 condensates is in any aspect of the cytoplasm of the cell. In some embodiments, the location of the one or more RBM20 condensates is in an organelle, non-membrane-bound cellular compartments (e.g., in or co-localize with stress granules), or particles of the cytoplasm, or based on an association thereto. In some embodiments, the location of the one or more RBM20 condensates describes the association of one or more RBM20 condensates with another cellular feature, such as the nucleus or centroid. In some embodiments, the location of the one or more RBM20 condensates is relative to another cellular feature, such as the nucleus or centroid. In some embodiments, the location of the one or more RBM20 condensates is based on a distance to another cellular feature, such as the nucleus or centroid. In some embodiments, the location of the one or more RBM20 condensates is in any aspect of the nucleus of the cell. In some embodiments, the location of the one or more RBM20 condensates is in and/or co-localize with paraspeckles.

In some embodiments, the distribution of the one or more RBM20 condensates (and/or RBM20 polypeptides) is the distribution of the one or more RBM20 condensates (and/or RBM20 polypeptides) in a portion of the cytoplasm, such as a cellular feature of the cytoplasm or a field of view. In some embodiments, the distribution of the one or more RBM20 condensates is the distribution of the one or more RBM20 condensates (and/or RBM20 polypeptides) relative to a cellular feature, such as the nucleus, an organelle, or particle in the cytoplasm. In some embodiments, the distribution of the one or more RBM20 condensates (and/or RBM20 polypeptides) is the distribution of the one or more RBM20 condensates (and/or RBM20 polypeptides) in a portion of the cytoplasm, such as a field of view, relative to a position therein. In some embodiments, the distribution of the one or more RBM20 condensates (and/or RBM20 polypeptides) is based on the distance of each RBM20 condensate (and/or each RBM20 polypeptide) to a reference point. In some embodiments, the distribution of the one or more RBM20 condensates (and/or RBM20 polypeptides) is the distribution of the one or more RBM20 condensates (and/or RBM20 polypeptides) in a portion of the nucleus. The distribution can be uniform (e.g., uniform RBM20 polypeptides across the cytoplasm and/or nucleus) or not uniform.

In some embodiments, the number of the one or more RBM20 condensates is the total number of RBM20 condensate in an aspect or area of a cell. In some embodiments, the number of the one or more RBM20 condensates is the total number of RBM20 condensates in the cytoplasm. In some embodiments, the number of the one or more RBM20 condensates is an estimate of the total number of RBM20 condensates in the cytoplasm based on measurements of less than the total cytoplasm. In some embodiments, the number of the one or more RBM20 condensates is the number of RBM20 condensates in a portion of the cytoplasm, such as in a field of view or in association with a cellular feature. In some embodiments, the number of the one or more RBM20 condensates is the total number of RBM20 condensates in the nucleus. In some embodiments, the number of the one or more RBM20 condensates is an estimate of the total number of RBM20 condensates in the nucleus based on measurements of less than the total nucleus. In some embodiments, the number of the one or more RBM20 condensates is the number of RBM20 condensates in a portion of the nucleus, such as in a field of view or in association with a cellular feature.

In some embodiments, the size of the one or more RBM20 condensates is based on the largest condensate-crossing dimension measurement, such as diameter, of each of the one or more RBM20 condensates. In some embodiments, the size of the one or more RBM20 condensates is based on the perimeter of each of the one or more RBM20 condensates. In some embodiments, the size of the one or more RBM20 condensates is based the cross-sectional area of each of the one or more RBM20 condensates, or an imaged representation thereof, such as from a top-down view. In some embodiments, the size of the one or more RBM20 condensates is based on the volume of each of the one or more RBM20 condensates. In some embodiments, the characteristic associated with the one or more RBM20 condensates is based on the average size of the one or more RBM20 condensates. In some embodiments, the characteristic associated with the one or more RBM20 condensates is based on the size distribution (such as d5, d10, d90, or d95) of the one or more RBM20 condensates.

In some embodiments, the characteristic associated with the one or more RBM20 condensates is based on the amount of the one or more RBM20 condensates. In some embodiments, the amount of the one or more RBM20 condensates is based on the number and size of the one or more RBM20 condensates. In some embodiments, the amount of the one or more RBM20 condensates is based on the number and size of the one or more RBM20 condensates in a portion of the cell. In some embodiments, the amount of the one or more RBM20 condensates is based on the number and size of the one or more RBM20 condensates in a portion of the cytoplasm. In some embodiments, the amount of the one or more RBM20 condensates is based on the number and size of the one or more RBM20 condensates in a portion of the nucleus).

In some embodiments, the ratio of the number of one or more RBM20 condensates and a reference condensate is the ratio of the number of one or more RBM20 condensates and another condensate not comprising a RBM20 polypeptide. In some embodiments, the ratio of the number of one or more RBM20 condensates and a reference condensate is the ratio of the number of one or more RBM20 condensates in a portion of the cytoplasm and one or more RBM20 condensates in another portion of the cytoplasm. In some embodiments, the ratio of the number of one or more RBM20 condensates and a reference condensate is the ratio of the number of one or more RBM20 condensates and a condensate comprising a RBM20 polypeptide located in the nucleus. In some embodiments, the characteristic associated with the one or more RBM20 condensates is based on the ratio of the amount of one or more RBM20 condensates and a reference condensate, such as another condensate not comprising the RBM20 polypeptide or a RBM20 condensate in the nucleus.

In some embodiments, the functional activity associated with the one or more RBM20 condensates is a functional activity associated with the RBM20 polypeptide. In some embodiments, the functional activity associated with the one or more RBM20 condensates is based on the functional activity associated with another polypeptide or nucleic acid other than the RBM20 polypeptide. In some embodiments, the functional activity associated with the one or more RBM20 condensates is based on the functional activity associated with another polypeptide or nucleic acid other than the RBM20 polypeptide within or associated with the RBM20 condensate.

In some embodiments, the composition of the one or more RBM20 condensates is the amount of the RBM20 polypeptide relative to at least one other component, such as a biomolecule, of the one or more RBM20 condensates. In some embodiments, the composition of the one or more RBM20 condensates is the presence, level, or absence of at least one component other than the RBM20 polypeptide, such as a polypeptide, nucleic acid, or compound. In some embodiments, the composition of the one or more RBM20 condensates is the amount of a wild type RBM20 polypeptide relative to a mutant RBM20 polypeptide within the condensate. In some embodiments, the composition of the one or more RBM20 condensates is the amount of a first mutant RBM20 polypeptide relative to a second mutant RBM20 polypeptide within the condensate. In some embodiments, the composition of the one or more RBM20 condensates is the amount of a first RBM20 polypeptide relative to a second RBM20 polypeptide, wherein the first and second RBM20 polypeptide have a composition difference, such as a difference in a post-translation modification.

In some embodiments, the co-localization of the one or more RBM20 condensates with a biomolecule is the co-localization of the one or more RBM20 condensates and another polypeptide and/or nucleic acid. In some embodiments, the biomolecule comprises a polypeptide. In some embodiments, the biomolecule comprises a nucleic acid, such as a DNA or RNA. In some embodiments, the biomolecule is associated with another condensate without RBM20 polypeptide component before admixing the compound, and/or before formation of the one or more RBM20 condensates (e.g., cytoplasmic RBM20 condensate comprising a mutant RBM20 polypeptide).

In some embodiments, the diffusion coefficient of a component of the one or more RBM20 condensates is the diffusion coefficient of the RBM20 polypeptide, such as a wild type or mutant RBM20 polypeptide, out of the one or more RBM20 condensates. In some embodiments, the diffusion coefficient of a component of the one or more RBM20 condensates is the diffusion coefficient of a component that is not RBM20 polypeptide (e.g., other protein, nucleic acid, or compound) out of the one or more RBM20 condensates.

In some embodiments, the stability of the one or more RBM20 condensates is the stability of the one or more RBM20 condensates over time, in the presence of a cellular activity, or in the presence of a compound. In some embodiments, the stability is based on the maintenance of, e.g., size, number, shape, or amount of the one or more RBM20 condensates.

In some embodiments, the dissolution or reduction in size of the one or more RBM20 condensates is based on the largest condensate-crossing dimension measurement, such as diameter, of each of the one or more RBM20 condensates. In some embodiments, the dissolution or reduction in size of the one or more RBM20 condensates is based on the perimeter of each of the one or more RBM20 condensates. In some embodiments, the dissolution or reduction in size of the one or more RBM20 condensates is based on the average size of the one or more RBM20 condensates. In some embodiments, the dissolution or reduction in size of the one or more RBM20 condensates is based on the size distribution (such as d5, dl 0, d90, or d95) of the one or more RBM20 condensates.

In some embodiments, the surface area of the one or more RBM20 condensates is an estimated surface area based on a dimensional feature, such as the perimeter, of each of the one or more RBM20 condensates.

In some embodiments, the sphericity of the one or more RBM20 condensates is based on how closely each of the one or more RBM20 condensates resembles a perfect sphere. In some embodiments, the sphericity of the one or more RBM20 condensates is an estimate sphericity based on a cross-section or top-down view of each of the one or more RBM20 condensates. In some embodiments, characteristic associated with one or more RBM20 condensates is the shape of each of the one or more RBM20 condensates. In some embodiments, the characteristic associated with one or more RBM20 condensates is the portion of the one or more RBM20 condensates having a shape type or meeting a shape parameter.

In some embodiments, the liquidity and/or solidification of the one or more RBM20 condensates is based on how the one or more RBM20 condensates fuse with each other, and/or changes in the structure, size, shape, sphericity, volume, number, and/or or surface area of each of the one or more RBM20 condensates over time. In some embodiments, the liquidity and/or solidification of the one or more RBM20 condensates is based on fiber formation.

In some embodiments, the location of the RBM20 polypeptide is in any aspect of the cytoplasm of the cell. In some embodiments, the location of the RBM20 polypeptide is in an organelle or particles of the cytoplasm, or based on an association thereto. In some embodiments, the location of the RBM20 polypeptide describes the association of the RBM20 polypeptide with another cellular feature, such as the nucleus. In some embodiments, the location of the RBM20 polypeptide is relative to another cellular feature, such as the nucleus or one or more RBM20 condensates.

In some embodiments, the amount of the RBM20 polypeptide or a precursor thereof is the amount of the precursor of the RBM20 polypeptide is a RNA, such as a mRNA. In some embodiments, the amount of the RBM20 polypeptide or a precursor thereof is based on the amount of the RBM20 polypeptide or a precursor thereof in a portion of the cell, such as the cytoplasm or the nucleus.

In some embodiments, the aggregation of the RBM20 polypeptide is the non-phase separated aggregation of the RBM20 polypeptide. In some embodiments, the characteristic associated with the RBM20 polypeptide is the level, such as amount or relative amount, of aggregation of the RBM20 polypeptide.

In some embodiments, the functional activity associated with the RBM20 polypeptide is based on the normal activity of the RBM20 polypeptide, such as a wild type RBM20 polypeptide, when located in the nucleus (e.g., RNA splicing). In some embodiments, the functional activity associated with the RBM20 polypeptide is based on function or characteristic of a cellular process, such as myocyte or cardiac function (e.g., calcium handling, ejection fraction, fractional shortening, QT or QTc interval), or cellular phenotype, such as sarcomere length/integrity.

In some embodiments, the post-translational modification status of the RBM20 polypeptide is based on the presence, level, or absence of at least one phosphorylation or methylation. In some embodiments, the post-translational modification status of the RBM20 polypeptide is based on the presence or absence of a phosphorylation of 5635. In some embodiments, the post-translational modification status of the RBM20 polypeptide is based on the presence or absence of a phosphorylation of R636S. In some embodiments, the post-translational modification status of the RBM20 polypeptide is based on the presence or absence of a phosphorylation of 5637. In some embodiments, the post-translational modification status of the RBM20 polypeptide is based on the presence or absence of a phosphorylation of 5635 and S637. In some embodiments, the post-translational modification status of the RBM20 polypeptide is based on the presence or absence of a phosphorylation of S635, R636S, and S637.

In some embodiments, the amount of a RBM20 polypeptide degradation product is the amount of a product, such as proteolytic products, of a RBM20 polypeptide. In some embodiments, the amount of the RBM20 polypeptide degradation product is based on the amount of the RBM20 polypeptide degradation product in a portion of the cell, such as the cytoplasm or the nucleus.

In some embodiments, the characteristic (e.g., functional activity) associated with the RBM20 polypeptide is based on the presence, absence, or level or Titan splicing.

In some embodiments, the methods described herein assess a plurality of characteristics associated with one or more RBM20 condensates in the cytoplasm of a cell and/or the RBM20 polypeptide in the cytoplasm of a cell. For example, in some embodiments, the characteristics comprise location of the one or more RBM20 condensates, distribution of the one or more RBM20 condensates and/or the RBM20 polypeptide, number of the one or more RBM20 condensates, size of the one or more RBM20 condensates, and ratio of the amount of one or more RBM20 condensates and a reference condensate. In some embodiments, the characteristics further comprise the functional activity associated with the one or more RBM20 condensates. In some embodiments, the characteristics comprise composition of the one or more RBM20 condensates, and co-localization of the one or more RBM20 condensates with a biomolecule. In some embodiments, the characteristics further comprise the functional activity associated with the one or more RBM20 condensates. In some embodiments, the characteristics comprise stability of the one or more RBM20 condensates, dissolution or reduction in size of the one or more RBM20 condensates, and surface area of the one or more RBM20 condensates. In some embodiments, the characteristics comprise sphericity of the one or more RBM20 condensates, liquidity of the one or more RBM20 condensates, and solidification of the one or more RBM20 condensates. In some embodiments, the characteristics comprise co-localization of the one or more RBM20 condensates with a biomolecule, and diffusion coefficient of a component of the one or more RBM20 condensates. In some embodiments, the characteristics comprise stability of the one or more RBM20 condensates, and dissolution or reduction in size of the one or more RBM20 condensates. In some embodiments, the characteristics comprise surface area of the one or more RBM20 condensates, sphericity of the one or more RBM20 condensates, liquidity of the one or more RBM20 condensates, and solidification of the one or more RBM20 condensates.

In some embodiments, the characteristics comprise location of the RBM20 polypeptide, and amount of the RBM20 polypeptide or a precursor thereof. In some embodiments, the characteristics comprise location of the RBM20 polypeptide, amount of the RBM20 polypeptide or a precursor thereof, and post-translational modification status of the RBM20 polypeptide. In some embodiments, the characteristics comprise location of the RBM20 polypeptide, amount of the RBM20 polypeptide or a precursor thereof, and the functional activity associated with the RBM20 polypeptide. In some embodiments, the characteristics comprise location of the RBM20 polypeptide, amount of the RBM20 polypeptide or a precursor thereof, post-translational modification status of the RBM20 polypeptide, and the functional activity associated with the RBM20 polypeptide.

ii. Determining Characteristics Associated with One or More RBM20 Condensates and/or the RBM20 Polypeptide in a Cell-Based Method

The characteristics associated with one or more RBM20 condensates and/or the RBM20 polypeptide in the cytoplasm of the cell, in some embodiments, may be determined based on any one or more of: e.g., assessment of the one or more RBM20 condensates and/or the RBM20 polypeptide in the cytoplasm or a portion of thereof; assessment of the one or more RBM20 condensates and/or the RBM20 polypeptide in any other location inside, outside, or associated with the cell, e.g., the nucleus, or a portion thereof; or assessment of another biomolecule, such as another RBM20 condensate component.

In some embodiments, determining the characteristic associated with the one or more RBM20 condensates and/or the RBM20 polypeptide is based on an assessment of at least one cell or at least one portion thereof. In some embodiments, the assessment is of a plurality of cells. In some embodiments, the portion of the cell is a field of view, such as a field of view of a microscope, or a portion thereof. In some embodiments, the portion of the cell is a defined area of an image of the cell, or a portion thereof. In some embodiments, the defined area is based on one or more cellular features (such as by fluorescent fusion protein expression, nuclear dye, or IF staining), e.g., the boundaries of the nucleus or cell membrane. In some embodiments, the defined area is arbitrarily defined, such as manually or by software. In some embodiments, determining the characteristic associated with the one or more RBM20 condensates and/or the RBM20 polypeptide is based on replicate assessments. In some embodiments, the replicate assessments are based on more than one portion of an image or more than one image. In some embodiments, determining the characteristic associated with the one or more RBM20 condensates and/or the RBM20 polypeptide is based on an average or distribution obtained from two or more portions of an image, two or more images, or two or more portions obtained from at least two or more images.

In some embodiments, determining a characteristic associated with the one or more RBM20 condensates and/or the RBM20 polypeptide is based on an imaging technique. In some embodiments, the imaging technique provides data to assess the characteristic associated with the one or more RBM20 condensates and/or the RBM20 polypeptide. In some embodiments, the imaging technique comprises obtaining of one or more images of a composition comprising a cell or a potion thereof. In some embodiments, the image is a two-dimensional image. In some embodiments, the image is a three-dimensional image or rendering thereof. In some embodiments, the imaging technique is coupled with another method feature useful for the methods described herein, such as a fluorescence activated cell sorter (FACS) technique, or a fluorescence-activated particle sorting (FAPS) technique.

In some embodiments, the methods described herein comprise imaging a sample or a portion thereof, such as a composition comprising a cell, via an imaging technique. In some embodiments, the imaging technique is a fluorescence imaging technique. In some embodiments, the imaging technique comprises a fluorescence imaging technique. In some embodiments, the imaging technique comprises a colorimetric and a fluorescence imaging technique. In some embodiments, the detected light is due to direct labeling of a target, such as incorporation or conjugation of a label into a compound or a RBM20 polypeptide. In some embodiments, the direct label comprises Dendra2, GFP, or mCherry. In some embodiments, the detected light is due to indirect labeling of a target, such a labeled probed that specifically binds to a RBM20 polypeptide, e.g., a labeled anti-RBM20 antibody or fragment thereof, a nuclear dye, or Annexin V luciferase that binds to phosphatidylserine (PS) exposed on the outer leaflet of cell membranes during apoptosis. In some embodiments, determining the characteristic comprises an immunofluorescence technique. In some embodiments, the methods described herein comprise use of a direct labeling technique and an indirect labeling technique.

In some embodiments, the method further comprises determining one or more cellular feature of the cell, such as in addition to a RBM20 condensate and/or a RBM20 polypeptide, if present, or a biomolecule, if present. One or ordinary skill in the art will readily recognize that cellular features can be determined in a number of ways. In some embodiments, the method further comprising contacting at least a portion of the composition or the cell with a stain. In some embodiments, the stain is a fluorescent stain. In some embodiments, the stain is a histochemical stain. In some embodiments, the stain is an immune-based stain, such as used in an immunohistochemistry or immunocytochemistry technique. In some embodiments, the stain allows for visualization of a cellular feature, if present, such as at least a portion of any one or more of the plasma or cell membrane, cytoplasm, cytoskeleton, nucleus, endoplasmic reticulum (rough and/or smooth), ribosome, Golgi body, lysosomes, mitochondrion, vacuole, or centrosome. In some embodiments, the methods described herein further comprising contacting at least a portion of the composition or the cell with a fixative.

In some embodiments, the method comprises analyzing the one or more images to assess the characteristic associated with one or more RBM20 condensates in the cytoplasm (or nucleus) of a cell and/or the RBM20 polypeptide in the cytoplasm (or nucleus) of a cell. In some embodiments, analyzing comprises mapping cell boundaries, or boundaries of a portion of a cell, such as an organelle. In some embodiments, analyzing comprises mapping boundaries of a condensate, such as an RBM20 condensate. In some embodiments, analyzing the images is completed and/or facilitated by analysis software. In some embodiments, the one or more images are compared, such as in a time course study. In some embodiments, analyzing comprises measuring signal (e.g., signal of fluorescent fusion protein, IF staining, or luminescence) intensity of one or more RBM20 condensates, the RBM20 polypeptide, a biomolecule (e.g., a co-localizing polypeptide that is not RBM20 polypeptide), a condensate that does not comprise RBM20 polypeptide, and/or a compound (e.g., a co-localizing compound with the condensate). For example, in some embodiments, analyzing comprises measuring the mCherry signal of a mutant R636S RBM20 polypeptide linked to a mCherry label, and the Dendra2 signal of a Nestin polypeptide linked to a Dendra2 label, within the RBM20 condensate boundary marked by mCherry. In some embodiments, analyzing further comprises calculating enrichment of a measured signal, such as a measured signal within a condensate boundary.

In some embodiments, the characteristic associated with one or more RBM20 condensates and/or the RBM20 polypeptide is determined based on the ratio of the number of cells having a RBM20 condensate and/or a RBM20 polypeptide with the characteristic and the number of cells not having the RBM20 condensate and/or the RBM20 polypeptide with the characteristic. In some embodiments, the characteristic associated with one or more RBM20 condensates and/or the RBM20 polypeptide is determined based on the number of cells having a RBM20 condensate and/or a RBM20 polypeptide with the characteristic. In some embodiments, the characteristic associated with one or more RBM20 condensates is determined based on the ratio of the number of RBM20 condensates with the characteristic within a cell (or within a field of view), such as the ratio of the number of RBM20 condensates comprising a biomolecule that is not RBM20 polypeptide.

In some embodiments, the characteristic associated with one or more RBM20 condensates and/or the RBM20 polypeptide is determined over a period of time, e.g., at two or more time points. In some embodiments, determining the characteristic associated with one or more RBM20 condensates and/or the RBM20 polypeptide comprises assessing the change in the characteristic over a period of time. For example, in some embodiments, cell proliferation, apoptosis, or necrosis is monitored over a period of time, such as by monitoring cell morphology, staining of cellular markers such as apoptosis markers (e.g., exposed PS), or propidium iodide (PI) staining. In some embodiments, the size, amount, location (e.g., cytoplasm or nucleus), and/or distribution (e.g., uniform across the cytoplasm or nucleus) of one or more RBM20 condensates and/or the RBM20 polypeptide is determined over a period of time after the RBM20 polypeptide is expressed in a cell (e.g., by transient transfection, or by doxycycline induction of expression with a TetOn system).

In some embodiments, the characteristic associated with one or more RBM20 condensates and/or the RBM20 polypeptide is determined using one or more measurements and/or techniques. In some embodiments, more than one characteristic associated with one or more RBM20 condensates and/or the RBM20 polypeptide is determined using one or more measurements and/or techniques.

In some embodiments, the methods described herein comprise techniques for, e.g., visualizing, analyzing, and/or quantifying condensates, polypeptide, and/or precursors thereof. Such techniques are well known by one of ordinary skill in the art. For example, encompassed herein are microscopy techniques for visualizing polypeptides, such as fluorescently labeled polypeptides, including those which are compatible with cell systems. Also encompassed herein are mass spectrometry (MS) techniques for analyzing the composition of polypeptides, including post-translation modifications, quantifying polypeptides, and studying the composition of RBM20 condensates (e.g., by cross-linking MS technique, or “XL-MS”). Also encompassed herein are functional assays for assessing cellular processes, cell viability, cell cytotoxicity, and cell proliferation. Also encompassed herein are enrichment and/or isolation techniques, e.g., centrifuge techniques for isolating cell fractions or affinity-based techniques for isolating polypeptides or nucleic acids. In some embodiments, the technique assesses the characteristic in one or more cells or in a defined area(s) of one or more cells. In some embodiments, the technique assesses one or more of the intensity, area, and condensate count in one or more cells or in a defined area(s) of one or more cells. In some embodiments, the technique assesses the number of cells with or without the characteristic. In some embodiments, the technique assesses the number of condensates within a cell with or without the characteristic. In some embodiments, the method comprises using a z-score to evaluate an assay and results therefrom. Z-scores, and uses thereof, are known in the art. See, e.g., Zhang et al., J Biomol Screen, 1999.

In some embodiments, the location of the one or more RBM20 condensates is determined by assessing the presence, absence, or level of the one or more RBM20 condensates in at least a portion of the cell, such as a cellular feature, e.g., the cytoplasm or nucleus, or in association with a portion of the cell. In some embodiments, determining the location of the one or more RBM20 condensates comprises determining a cellular feature.

In some embodiments, the distribution of the one or more RBM20 condensates and/or the RBM20 polypeptide is determined by assessing the presence, absence, or level of the one or more RBM20 condensates and/or the RBM20 polypeptide relative to a cellular feature or in a portion of a cell, such as a field of view. In some embodiments, determining the distribution of the one or more RBM20 condensates comprises and/or the RBM20 polypeptide determining the distribution of the one or more RBM20 condensates and/or the RBM20 polypeptide relative to an arbitrarily determined point of a cell or an image thereof.

In some embodiments, the number of the one or more RBM20 condensates is determined by assessing the total number of RBM20 condensates in the cell, such as the cytoplasm or the nucleus. In some embodiments, the number of the one or more RBM20 condensates is determined by assessing the number of RBM20 condensates in a portion, such as a field of view, of the cell, e.g., the cytoplasm or the nucleus. In some embodiments, the number of the one or more RBM20 condensates is determined by estimating the total number of RBM20 condensates in the cell, such as the cytoplasm or the nucleus, or a portion thereof, based on measurements of less than the total of the cell.

In some embodiments, the size of the one or more RBM20 condensates is determined by assessing the largest condensate-crossing dimension measurement, such as diameter, of each of the one or more RBM20 condensates. In some embodiments, the size of the one or more RBM20 condensates is determined by assessing the perimeter of each of the one or more RBM20 condensates. In some embodiments, the size of the one or more RBM20 condensates is determined by assessing the cross-sectional area of each of the one or more RBM20 condensates, or an imaged representation thereof, such as from a top-down view. In some embodiments, the size of the one or more RBM20 condensates is determined by a particle size measuring technique, such as a dynamic light scattering technique.

In some embodiments, the ratio of the number of one or more RBM20 condensates and a reference condensate is determined by assessing the number of RBM20 condensates in the cytoplasm of the cell and the number of reference condensates, wherein the reference condensate does not comprise the RBM20 polypeptide. In some embodiments, the reference condensate is in the cytoplasm of a cell. In some embodiments, the reference condensate is in the nucleus of a cell. In some embodiments, the ratio of the number of one or more RBM20 condensates and a reference condensate is determined by assessing the number of RBM20 condensates in the cytoplasm of the cell and the number of reference condensates in the nucleus, such as a nuclear condensate comprising a RBM20 polypeptide. In some embodiments, the ratio of the number of one or more RBM20 condensates and a reference condensate is determined by assessing the number of RBM20 condensates comprising a first RBM20 polypeptide and the number of reference condensates, wherein the reference condensate is a condensate comprising a second RMB20 polypeptide.

In some embodiments, the amount of one or more RBM20 condensates is determined by assessing the number and size of the one or more RBM20 condensates. In some embodiments, the amount of one or more RBM20 condensates is determined in a portion of a cell, such as the cytoplasm or nucleus, or a portion of an image of the cell.

In some embodiments, the functional activity associated with one or more RBM20 condensates is determined by assessing the precursor, intermediate, or product of a cellular process associated with the one or more RBM20 condensates. For example, in some embodiments, the functional activity associated with one or more RBM20 condensates is determined by assessing for the presence, absence, or level of an enzymatic activity, or product thereof. In some embodiments, the functional activity associated with one or more RBM20 condensates is determined by assessing for the presence, absence, or level of a Titan isoform or precursor thereof. In some embodiments, the functional activity associated with one or more RBM20 condensates is determined by assessing for the presence, absence, or level of one or more of a polypeptide or nucleic acid, such as DNA or RNA.

In some embodiments, the composition of one or more RBM20 condensates is determined by assessing for the presence, absence, or level of at least one other component of or associated with one or more RBM20 condensates. In some embodiments, determining the composition of the one or more RBM20 condensates comprises determining at least one other components of or associated with one or more RBM20 condensates relative to the RBM20 polypeptide. In some embodiments, the other component is assessed, such as measured, directly or indirectly. In some embodiments, the other component is assessed using a mass spectrometry technique, such as APEX, or XL-MS. In some embodiments, the other component (e.g., the presence of absence) is assessed using a FAPS technique. In some embodiments, the other component is assessed using a labeling technique, such as directly conjugating a label to the other component or using an immuno-label. In some embodiments, the one or more RBM20 condensates is isolated and/or enriched from other cellular components. In some embodiments, the one or more RBM20 condensates is not isolated and/or enriched from other cellular components, e.g., assessing in situ. The composition of one or more RBM20 condensates can be fixed or not fixed prior to determination.

In some embodiments, the co-localization of the one or more RBM20 condensates with a biomolecule, e.g., a RNA or a protein, such as actin, or a compound, is determined by assessing for the presence, absence, or level of the biomolecule (or the compound) in or associated with the one or more RBM20 condensates. In some embodiments, the biomolecule (or the compound) is assessed, such as measured, directly or indirectly. In some embodiments, the biomolecule is assessed using a mass spectrometry technique, such as APEX, or XL-MS. In some embodiments, the biomolecule (e.g., the presence of absence) is assessed using a FAPS technique. In some embodiments, the biomolecule is assessed using a labeling technique, such as directly conjugating a label to the other component or using an immuno-label, such as used in an IF technique. In some embodiments, the one or more RBM20 condensates are isolated and/or enriched from other cellular components, and then the presence, absence, or level of the biomolecule in or associated with the one or more RBM20 condensates is assessed. In some embodiments, the one or more RBM20 condensates are not isolated and/or enriched from other cellular components, e.g., assessing in situ. The one or more RBM20 condensates, or the cell comprising one or more RBM20 condensates, can be fixed or not fixed prior to determination.

In some embodiments, the diffusion coefficient of a component, such as a RBM20 polypeptide, of the one or more RBM20 condensates is determined based on a fluorescence correlation spectroscopy technique.

In some embodiments, the stability of the one or more RBM20 condensates is determined based on a fusion or fission technique. In some embodiments, the stability of the one or more RBM20 condensates is determined based on changes in the structure of each of the one or more RBM20 condensates over time, in the presence of a cellular activity, or in the presence of a compound. In some embodiments, the stability is of the one or more RBM20 condensates is determined based on assessing any one or more of the size, shape, sphericity, volume, or surface area of each of the one or more RBM20 condensates.

In some embodiments, the dissolution or reduction in size of the one or more RBM20 condensates is determined based on changes in the structure of each of the one or more RBM20 condensates over time, in the presence of a cellular activity, or in the presence of a compound. In some embodiments, the dissolution or reduction in size of the one or more RBM20 condensates is determined based on assessing any one or more of the size, shape, sphericity, volume, number (including absence thereof), or surface area of each of the one or more RBM20 condensates.

In some embodiments, the surface area of the one or more RBM20 condensates is determined based on estimating a surface area using measured parameters (e.g., perimeter, largest condensate-crossing dimension measurement) of each of the one or more RBM20 condensates.

In some embodiments, the sphericity of the one or more RBM20 condensates is determined based on a 3D lattice light sheet technique. In some embodiments, the sphericity of the one or more RBM20 condensates is determined based on a 2D imaging technique. In some embodiments, the sphericity of the one or more RBM20 condensates is determined based on a cross-section or top-down view of each of the one or more RBM20 condensates.

In some embodiments, the liquidity and/or solidification of one or more RBM20 condensates is determined based on a fusion or fission technique. In some embodiments, the liquidity and/or solidification of one or more RBM20 condensates is determined based on changes in the structure of each of the one or more RBM20 condensates over time. In some embodiments, the liquidity and/or solidification of one or more RBM20 condensates is determined based on assessing changes in any one or more of the size, shape, sphericity, volume, number, or surface area of each of the one or more RBM20 condensates. In some embodiments, the liquidity and/or solidification of one or more RBM20 condensates is determined based on fiber formation and extent thereof.

In some embodiments, the location of the RBM20 polypeptide is determined by an imaging technique, such as a fluorescent imaging technique. In some embodiments, the location of the RBM20 polypeptide is determined directly (such as using a labeled RBM20 polypeptide) or indirectly (such as using a labeled probe, e.g., a labeled anti-RBM20 antibody or fragment thereof). In some embodiments, RBM20 polypeptide is isolated from a fraction of the cell, such as the cytoplasm or the nucleus, and that fraction of the cell is subsequently assessed for presence, absence, or level of the RBM20 polypeptide.

In some embodiments, the amount of the RBM20 polypeptide or a precursor thereof, such as RNA, is determined by an imaging technique, such as a fluorescent imaging technique. In some embodiments, the amount of the RBM20 polypeptide is determined directly (such as using a labeled RBM20 polypeptide) or indirectly (such as using a labeled probe, e.g., a labeled anti-RBM20 polypeptide). In some embodiments, RBM20 polypeptide or a precursor thereof is isolated from a fraction of the cell, such as the cytoplasm or the nucleus, and that fraction of the cell is subsequently assessed for the amount of the RBM20 polypeptide or a precursor thereof. Any known methods in the art can be used to quantify the amount of the RBM20 polypeptide or a precursor thereof, such as western blot, immunoprecipitation (IP), immunofluorescence (IF) staining, in situ, FISH, northern blot, or qPCR.

In some embodiments, the condensate partitioning of the RBM20 polypeptide into the one or more RBM20 condensates is determined based on the amount of the RBM20 polypeptide out of one or more RBM20 condensates as compared to the amount of the RBM20 polypeptide in or associated with one or more RBM20 condensates. In some embodiments, the condensate partitioning into the one or more RBM20 condensates is determined for one or more other biomolecule that is not RBM20 polypeptide (e.g., other polypeptide, or nucleic acid) or for a compound. In some embodiments, condensate partitioning can be determined by an imaging technique, such as a fluorescent imaging technique, for example, by directly labeling a biomolecule (e.g., RBM20 polypeptide) or compound, or indirectly such as using a labeled probe (e.g., antibody staining).

In some embodiments, the functional activity associated with the RBM20 polypeptide is based on the normal activity of the RBM20 polypeptide when located in the nucleus, such as the state of Titan splicing. In some embodiments, the functional activity associated with the RBM20 polypeptide is determined by a Titan splicing assay.

In some embodiments, the aggregation of the RBM20 polypeptide is determined based on the presence, absence, or level of non-phase separated RBM20 polypeptide aggregation.

In some embodiments, the post-translational modification status of the RBM20 polypeptide is determined based on the presence, absence, or level of a RBM20 polypeptide PTM, such as one or more phosphorylation or methylation. In some embodiments, determining the PTM status of the RBM20 polypeptide comprises enriching for one or more species of a modified RBM20 polypeptide. In some embodiments, the presence, absence, or level of post-translational modification can be determined by IF staining, such as using anti-phospho antibody.

In some embodiments, the amount of the RBM20 polypeptide degradation product is determined based on a protein measurement technique, such as a protein measurement technique described herein. In some embodiments, the amount of the RBM20 polypeptide degradation product is determined based on a mass spectrometry technique or a western blot technique. In some embodiments, the protein measurement technique is quantitative.

In some embodiments, the characteristic associated with one or more RBM20 condensates and/or the RBM20 polypeptide (e.g., composition) is determined based on immunofluorescence, fluorescence in situ hybridization (FISH), mass spectrometry (MS), RNA-seq, or NMR spectroscopy, etc.

In some embodiments, the characteristic associated with one or more RBM20 condensates and/or the RBM20 polypeptide (e.g., liquidity and/or solidification) is determined based on a fluorescence recovery after photobleaching (FRAP) technique. In some embodiments, the FRAP technique is performed to assess complete condensates, for example, to measure the exchange of components of the one or more RBM20 condensates with the cytoplasm or nucleus. In some embodiments, the FRAP technique is performed to assess half condensates, for example, to measure the internal dynamics within the one or more RBM20 condensates.

In some embodiments, the characteristic associated with one or more RBM20 condensates and/or the RBM20 polypeptide is determined based on photo-conversion of a fluorophore, such as Dendra2.

In some embodiments, the characteristic associated with one or more RBM20 condensates and/or the RBM20 polypeptide is determined based on a fluorescence correlation spectroscopy (FCS) technique.

In some embodiments, the characteristic associated with one or more RBM20 condensates and/or the RBM20 polypeptide is determined based on temperature responsiveness of the one or more RBM20 condensates and/or the RBM20 polypeptide.

In some embodiments, the characteristic associated with one or more RBM20 condensates and/or the RBM20 polypeptide is determined based on tracking condensate fusion and/or fission, such as fusion and/or fission in a cell.

In some embodiments, the characteristic associated with one or more RBM20 condensates and/or the RBM20 polypeptide is determined based on a 3D lattice light sheet technique.

In some embodiments, the characteristic associated with one or more RBM20 condensates and/or the RBM20 polypeptide is determined based on a super resolution imaging technique, such as stimulated emission depletion (STED) microscopy, stochastic optical reconstruction microscopy (STORM), or photoactivated localization microscopy (PALM), or a hybrid thereof.

In some embodiments, characteristic associated with one or more RBM20 condensates and/or the RBM20 polypeptide is determined based on ultraviolet-visible (UV-Vis) spectroscopy, small-angle x-ray scattering, or Static and Dynamic Light Scattering (SLS/DLS) technique. For example, light scattering methods like dynamic light scattering (DLS), static light scattering (STS) and small-angle light scattering (SLS) can be used to determine the size and shape of condensates. Also see various in vitro and in cell condensate analysis methods described in Basturea, G. N. (“Biological Condensates,” MATER METHODS 2019; 9:2794).

In some embodiments, the characteristic associated with one or more RBM20 condensates and/or the RBM20 polypeptide is determined in the presence of a cellular stress. For example, in some embodiments, the cellular stress is one or more of hypoxia, oxidative stress, apoptosis stress (e.g., staurosporine), energy stress (OXPHOS or glycolysis), ATP depletion, temperature, such as heat, or mechanical stress, e.g., such as occurs during irregular or excessive frequency of heart beating.

In some embodiments, the characteristic associated with one or more RBM20 condensates and/or the RBM20 polypeptide is determined in the presence of beating/contraction in a cell, such as a cardiomyocyte. In some embodiments, the characteristic associated with one or more RBM20 condensates and/or the RBM20 polypeptide is determined based on calcium handling, ejection fraction, fractional shortening, QT or QTc interval of a cardiomyocyte. In some embodiments, the characteristic associated with one or more RBM20 condensates and/or the RBM20 polypeptide is determined based on sarcomere length/integrity. In some embodiments, the characteristic associated with one or more RBM20 condensates and/or the RBM20 polypeptide is determined in the presence of external mechanical force, such as applied using an atomic force microscopy technique.

In some embodiments, the modulation in the characteristic is based on a change in the presence, absence, or level of the one or more RBM20 condensates and/or the RBM20 polypeptide. In some embodiments, the modulation in the characteristic is based on a decrease in the number of the one or more RBM20 condensates in the cytoplasm (or nucleus) of the cell. In some embodiments, the modulation in the characteristic is based on an increase in the number of the one or more RBM20 condensates in the cytoplasm (or nucleus) of the cell. In some embodiments, the modulation in the characteristic is based on the formation of the one or more RBM20 condensates, such as the formation of the one or more RBM20 condensates in the cytoplasm or nucleus of the cell. In some embodiments, the modulation in the characteristic is based on a decrease in the size of the one or more RBM20 condensates in the cytoplasm (or nucleus) of the cell. In some embodiments, the modulation in the characteristic is based on an increase in the size of the one or more RBM20 condensates in the cytoplasm (or nucleus) of the cell.

In some embodiments, the modulation in the characteristic is based on a decrease in the amount of the RBM20 polypeptide or a precursor thereof in the cytoplasm (or nucleus) of the cell. In some embodiments, the modulation in the characteristic is based on an increase in the amount of the RBM20 polypeptide or a precursor thereof in the cytoplasm (or nucleus) of the cell. In some embodiment, the modulation in the characteristic is based on an increase in the presence of one or more post-translation modification on a RBM20 polypeptide. In some embodiment, the modulation in the characteristic is based on a reduction in the presence of one or more post-translation modification on a RBM20 polypeptide.

In some embodiments, the modulation in the characteristic is based on an increase in the amount of one or more RBM20 condensates in the nucleus of the cell. In some embodiments, the modulation in the characteristic is based on an increase in the amount of the RBM20 polypeptide in the nucleus of the cell.

In some embodiments, the modulation in the characteristic is based on a decrease in the functional activity associated with the one or more RBM20 condensates and/or the RBM20 polypeptide in a portion of the cell, such as the cytoplasm (or nucleus) of the cell. In some embodiments, the modulation in the characteristic is based on an increase in the functional activity associated with the one or more RBM20 condensates and/or the RBM20 polypeptide in a portion of the cell, such as the cytoplasm (or nucleus) of the cell.

In some embodiments, the modulation in the characteristic is based on a change in the presence, absence, or level of a biomolecule that is not RBM20 polypeptide (e.g., other polypeptide, or nucleic acid) within the one or more RBM20 condensates. In some embodiments, the modulation in the characteristic is based on a decrease in the amount of the biomolecule within the one or more RBM20 condensates in the cytoplasm (or nucleus) of the cell. In some embodiments, the modulation in the characteristic is based on an increase in the amount of the biomolecule within the one or more RBM20 condensates in the cytoplasm (or nucleus) of the cell.

In some embodiments, the characteristic associated with the one or more RBM20 condensates and/or the RBM20 polypeptide is determined based a characteristic associated with a heart or heart tissue, such as the size of the heart of features thereof, and muscle contraction (e.g., ejection fraction, fractional shortening, QT or QTc interval). In some embodiments, the characteristic associated with the one or more RBM20 condensates and/or the RBM20 polypeptide is determined based on Titan splicing.

In some embodiments, the methods described herein comprises determining the specificity of a compound for modulating a characteristic associated with one or more RBM20 condensates and/or the RBM20 polypeptide. For example, in some embodiments, the method comprises determining modulation of a characteristic associated with a first RBM20 condensates and a second RBM20 condensate, wherein the compound modulates the characteristic in the first RBM20 condensate and not the second RBM20 condensate. In some embodiments, the first RBM20 condensate comprises a wild type RBM20 polypeptide and the second RBM20 condensate comprises a mutant RBM20 polypeptide. In some embodiments, the first RBM20 condensate comprises a mutant RBM20 polypeptide and the second RBM20 condensate comprises a wild type RBM20 polypeptide. In some embodiments, the first RBM20 condensate is in the nucleus and the second RBM20 condensate is in the cytoplasm. In some embodiments, the first RBM20 condensate is in the cytoplasm and the second RBM20 condensate is in the nucleus. In some embodiments, the first RBM20 condensate comprises a first mutant RBM20 polypeptide and the second RBM20 condensate comprises a second mutant RBM20 polypeptide, wherein the first and second mutant RBM20 polypeptides are different.

In some embodiments, the method comprises determining modulation of a characteristic associated with a RBM20 condensate and a non-RBM20 condensate (a condensate that does not comprise a RBM20 polypeptide), wherein the compound modulates the characteristic in the RBM20 condensate and not the non-RBM20 condensate. In some embodiments, the method comprises determining modulation of a characteristic associated with a RBM20 condensate and a non-RBM20 condensate (a condensate that does not comprise a RBM20 polypeptide), wherein the compound modulates the characteristics of both condensates. For example, in some embodiments, the compound relocates a biomolecule that is not an RBM20 polypeptide which is sequestered in the RBM20 condensate under a disease or stress status back to the non-RBM20 condensate or cellular location (e.g., cytoplasm or nucleus) where it should have resided under a heathy or non-stress status. In some embodiments, the compound relocates the RBM20 polypeptide from the RBM20 condensate in the cytoplasm (e.g., under a disease or stress status) back to a condensate in the nucleus where it should have resided under a healthy or non-stress status.

In some embodiments, the method comprises determining modulation of a characteristic associated with a first RBM20 condensates and a second RBM20 condensate, wherein the compound modulates the characteristic in the first RBM20 condensate and the second RBM20 condensate. In some embodiments, the first RBM20 condensate comprises a wild type RBM20 polypeptide and the second RBM20 condensate comprises a mutant RBM20 polypeptide. In some embodiments, the first RBM20 condensate comprises a first mutant RBM20 polypeptide and the second RBM20 condensate comprises a second mutant RBM20 polypeptide, wherein the first and second mutant RBM20 polypeptides are different. In some embodiments, the first RBM20 condensate is in the cytoplasm and the second RBM20 condensate is in the nucleus. In some embodiments, both RBM20 condensates are in the nucleus. In some embodiments, both RBM20 condensates are in the cytoplasm.

In some embodiments, the method comprises determining one or more of cell viability, cell cytotoxicity, and cell proliferation. In some embodiments, cell viability is assessed via a marker that correlates to the number of living cells and/or cellular functions. In some embodiments, cell viability is assessed via levels of ATP, such as using the titer glo method. In some embodiments, cell viability is assessed via cell metabolism, such as using the RealTime-Glo™ MT method. In some embodiments, cytotoxicity (e.g., apoptosis or necrosis) is assessed via PS exposure or loss of membrane integrity, such as using RealTime-Glo™ Annexin V apoptosis and necrosis assay. In some embodiments, cell viability is assessed via translation, such as using puromycin incorporation. In some embodiments, cell cytotoxicity is assessed via a marker that correlated to the number of dead and/or dying cells. In some embodiments, cell cytotoxicity is assessed via membrane integrity, such as using cell-permeable dyes. In some embodiments, cell cytotoxicity is assessed via apoptotic or necrotic death markers, such as using annexin V or TUNEL, or by propidium iodide (PI) staining. Any apoptosis assays known in the art can be employed herein, such as DNA ladder formation, analysis of DNA fragmentation by TUNEL, enzyme-linked immunosorbent assay for histone/DNA fragment, poly-ADP-ribose-polymerase cleavage assay, and terminal deoxynucleotidyl transferase-mediated dUTP nick-end-labeling assay. Several apoptosis markers can be assessed. For example, cleavage of anti-apoptotic Bcl-2 family proteins can be assessed by western blot of protein cleavage. Caspase activation can be assessed by colorimetric/fluorometric substrate-based assays in microtiter plates, detection of cleavage of the fluorometric substrate in flow cytometry/microscopy or by microtiter plates analysis, western blot analysis of pro- and active caspase, flow cytometry/microscopy analysis with antibodies specifically recognizing the active form of caspases, or microplate spectrophotometry analysis with antibodies specifically recognizing the active form of caspases. Caspase substrate (PARP) cleavage can be assessed by microplate spectrophotometry analysis with antibodies specific for cleaved PARP, or western blot analysis of cleaved PARP. Non-caspase proteases (cathepsins and calpain) activation can be assessed by colorimetric/fluorometric substrate-based assays in microtiter plates. Mitochondrial transmembrane potential (Δ ψm) decrease can be assessed by flow cytometry/microscopy/microplate spectrophotometry analysis with Δ ψm sensitive probes, or oxygen consumption studies. Cytochrome C release can be assessed by western blot or antibody-based microscopy analysis of the presence of cytochrome C in the cytosol. Increase of sub G1 population can be assessed by flow cytometry analysis of sub G1 peak. Nuclear condensation can be assessed by flow cytometry or microscopy analysis or of chromatin condensation. Membrane blebbing can be assessed by light microscopy, or western blot analysis of cleaved substrate (gelsolin, ROCK1). In some embodiments, cell proliferation is assessed via cell confluency. In some embodiments, cell proliferation is assessed via a proliferation marker, such as a marker for measuring Ki67, eFluor dyes, and/or nuclear dyes.

iii. Cells and Compositions Comprising the Cell

In some embodiments, the composition comprising a cell described herein comprises a plurality of the cells. In some embodiments, the composition comprising a cell described herein comprises a plurality of the cells, wherein the plurality of the cells are selected based on expression level of a RBM20 polypeptide, such as selected based on having a similar expression level of a RBM20 polypeptide. In some embodiments, the composition comprising a cell described herein comprises a plurality of the cells, wherein the plurality of the cells are clones derived from a single cell.

In some embodiments, the cell comprises the one or more RBM20 condensates, such as comprises the one or more RBM20 condensates prior to contacting with a compound as described in the methods herein. In some embodiments, the one or more RBM20 condensates form in the cell after the compound contacts the composition. In some embodiments, the one or more RBM20 condensates are capable of forming in the cell in the absence of a compound, such as in the instance when the cell is contacted with the compound after initiating expression of a RBM20 polypeptide that forms cytoplasmic RBM20 condensates in the absence of the compound. In some embodiments, the one or more RBM20 condensates form in the cell upon stress.

In some embodiments, the cell expresses a RBM20 polypeptide, such as a labeled RBM20 polypeptide, at about the level of endogenous RBM20 polypeptide expression. In some embodiments, the cell expresses a RBM20 polypeptide, such as a labeled RBM20 polypeptide, at about the level of RBM20 polypeptide expression in a reference cell. In some embodiments, the cell expresses a RBM20 polypeptide, such as a labeled RBM20 polypeptide, at above the level of endogenous RBM20 polypeptide expression. In some embodiments, the cell expresses a RBM20 polypeptide, such as a labeled RBM20 polypeptide, at about the level of endogenous RBM20 polypeptide expression, wherein the cell is altered to reduce the level of the degradation of the RBM20 polypeptide. In some embodiments, the cell expresses a RBM20 polypeptide, such as a labeled RBM20 polypeptide, below the level of endogenous RBM20 polypeptide expression. In some embodiments, cells used in the methods described herein are sorted based on expression level of a RBM20 polypeptide. In some embodiments, the cell expresses a first RBM20 polypeptide and a second RBM20 polypeptide, wherein the first and second RBM20 polypeptides are different, e.g., the first RBM20 polypeptide is a wild type RBM20 polypeptide and the second RBM20 polypeptide is a mutant RBM20 polypeptide or the first RBM20 polypeptide is a first mutant RBM20 polypeptide and the second RMB20 polypeptide is a second mutant RBM20 polypeptide, wherein the first and second mutant RBM20 polypeptides are different. In some embodiments, the cell expresses a wild type RBM20 polypeptide and a mutant RBM20 polypeptide.

In some embodiments, the cell comprises a construct comprising a nucleic acid sequence of a RBM20 polypeptide. In some embodiments, the construct comprises a promoter. In some embodiments, the promoter is a cytomegalovirus (CMV) promoter. In some embodiments, the promoter is an inducible promoter, such as a promoter controlled by the presence of tetracycline or doxycycline. In some embodiments, the cell comprises one or more constructs encoding a wild type RBM20 polypeptide and a mutant RBM20 polypeptide. In some embodiments, the nucleic acids encoding a first RBM20 polypeptide and a second RBM20 polypeptide are on the same vector, under the same promoter control or under different promoter controls. In some embodiments, the nucleic acids encoding a first RBM20 polypeptide and a second RBM20 polypeptide are on different vectors. The first RBM20 polypeptide and the second RBM20 polypeptide can be expressed with the same or different amounts.

In some embodiments, the cell has endogenous RBM20 polypeptide knocked down or knocked out, such as via CRISPR, and the cell expresses a heterologous RBM20 polypeptide, such as a labeled RBM20 polypeptide and/or a mutant RBM20 polypeptide. In some embodiments, the cell comprises a modified endogenous locus of a RBM20 polypeptide. In some embodiments, the endogenous locus of a RBM20 polypeptide is modified adjust the expressed RBM20 polypeptide, e.g., to create a mutant RBM20 polypeptide. In some embodiments, the endogenous locus of a RBM20 polypeptide is modified to insert a label.

In some embodiments, the cell has endogenous RBM20 polypeptide knocked down or knocked out using the dTAG system for immediate and target-specific protein degradation (Nabet et al., Nature Chemical Biology, 2018). Briefly, this system pairs the degrader of FKBP with expression of FKBP in-frame with RBM20, and the presence of dTAG can recruit E3 ubiquitin ligase to RBM20, marking it for proteosomal degradation. Such cell lines can be constructed by CRISPR-mediated locus-specific knock-in.

In some embodiments, the cell is a mammalian cell. In some embodiments, the cell is a primate cell. In some embodiments, the cell is a primate cell. In some embodiments, the cell is a human cell. In some embodiments, the cell is a rat cell. In some embodiments, the cell is a mouse cell.

In some embodiments, the cell is a model of a cardiac cell type, such as a model for a feature or characteristic of a cardiac cell type. In some embodiments, the cell is a cardiomyocyte. In some embodiments, the cardiomyocyte is a H9C2 cell. In some embodiments, the cardiomyocyte is a patient-derived cardiomyocyte. In some embodiments, the cardiomyocyte is an induced pluripotent stem (iPS) cell, such as a human iPS, e.g., a KOLF cell or a WTC-11 cell, differentiated to a cardiomyocyte. In some embodiments, the cardiomyocyte is a stem cell, such as a human stem cell, differentiated to a cardiomyocyte. In some embodiments, the cell is a patient-derived cardiomyocyte, such as an AC-16 cell.

In some embodiments, the cell is a HeLa cell. In some embodiments, the cell is a U2OS (human bone osteosarcoma epithelial) cell. In some embodiments, the cell is an induced pluripotent stem (iPS) cell. In some embodiments, the cell is a human iPS cell, such as a KOLF cell or a WTC-11 cell). In some embodiments, the cell is a stem cell, such as a human stem cell.

In some embodiments, the cell is homozygous for alleles encoding the RBM20 polypeptide. In some embodiments, the cell is heterozygous for alleles encoding the RBM20 polypeptide.

iv. Cell-Based References

In some embodiments, the methods described herein determine a modulation in a characteristic associated with one or more RBM20 condensates and/or the RBM20 polypeptide as compared to a reference.

In some embodiments, the reference is an established value for a characteristic. In some embodiments, the reference comprises the composition comprising the cell admixed with a vehicle control. In some embodiments, the reference comprises the composition comprising the cell admixed with a reference compound, such as a negative or positive control reference compound. In some embodiments, the reference comprises the composition comprising the cell admixed with the compound, wherein for the reference the characteristic associated with one or more RBM20 condensates and/or the RBM20 polypeptide is determined at a different time. In some embodiments, the reference is a cell comprising a different RBM20 polypeptide, such as a wild type or mutant RBM20 polypeptide. In some embodiments, the reference is a cell comprising a different level of a RBM20 polypeptide. In some embodiments, the reference is a cell comprising a RBM20 polypeptide having a different post-translation modification status. In some embodiments, the reference is a cell that is not induced to express RBM20 polypeptide or does not express RBM20 polypeptide. In some embodiments, the reference is a cell under stress or disease condition. In some embodiments, the reference is a cell under non-stress or healthy condition. In some embodiments, the reference is a cell comprising a RBM20 polypeptide tagged with a cellular location tag, such as nuclear localization signal.

In some embodiments, the modulation is assessed via a change in the degree of a characteristic described herein, such as an increase, decrease, or no change. In some embodiments, more than one reading or replicate is analyzed to determine the modulation of the characteristic. In some embodiments, the modulation measurement is normalized to that of a reference. In some embodiments, statistical methods are used to assess the significance of a modulation, such as a p-value.

v. Exemplary Cell-Based Methods

In some embodiments, described herein is a method of identifying a compound that reduces the size and/or number of RBM20 condensates in the cytoplasm of a cell, the method comprising: (a) determining the size and/or number of the RBM20 condensates in at least a portion of the cytoplasm of the cell subjected to the compound; and (b) comparing the size and/or number of the RBM20 condensates with a reference, thereby identifying the compound that reduces the size and/or number of the RBM20 condensates in the cytoplasm of the cell. In some embodiments, the compound reduces the number of RBM20 condensates. In some embodiments, the compound reduces the size of RBM20 condensates. In some embodiments, the cell is modified using CRISPR to knockout the endogenous RBM20 gene and is modified to express a heterologous RBM20 polypeptide comprising a fluorescent label. In some embodiments, the RBM20 polypeptide is a mutant RBM20 polypeptide, such as a R636S RBM20 polypeptide. In some embodiments, the RBM20 polypeptide is a wild type polypeptide. In some embodiments, the reference is the cell comprising a mutant RBM20 polypeptide contacted with a vehicle control. In some embodiments, the reference is the cell comprising a wild type RBM20 polypeptide contacted with the compound. In some embodiments, the size and/or number of the RBM20 condensates is determined using an imaging technique, such as a fluorescent imaging technique.

In some embodiments, described herein is a method of identifying a compound that prevents formation or growth of one or more condensates comprising a RBM20 polypeptide (“RBM20 condensates”) in the cytoplasm of a cell, the method comprising: (a) combining the compound and a composition comprising the cell, wherein (i) the cell comprises the one or more RBM20 condensates, and/or (ii) the one or more RBM20 condensates form after the compound contacts the composition; and (b) obtaining a first measurement of the size and/or number of the one or more RBM20 condensates in at least a portion of the cytoplasm of the cell; and (c) comparing the first measurement to a reference, thereby identifying a compound that prevents formation or growth of the one or more RBM20 condensates in the cytoplasm of the cell. In some embodiments, the reference comprises an aliquot of the composition comprising the cell admixed with a control agent. In some embodiments, the reference is a second measurement of the size and/or number of the one or more RBM20 condensates in at least a portion of the cytoplasm of the cell, and wherein the second measurement is taken at a different time than the first measurement. In some embodiments, the method further comprises subjecting the cell to a condition that promotes formation of the one or more RBM20 condensates. In some embodiments, the condition that promotes formation of the one or more RBM20 condensates is one or more of an increase in the expression level or amount of the RBM20 polypeptide, increase expression level or amount of a RBM20 condensate scaffold molecule, or an increase in the level of a PTM. In some embodiments, the cell is modified using CRISPR to knockout the endogenous RBM20 gene and is modified to express a heterologous RBM20 polypeptide comprising a fluorescent label. In some embodiments, the RBM20 polypeptide is a mutant RBM20 polypeptide, such as a R636S RBM20 polypeptide. In some embodiments, the RBM20 polypeptide is a wild type polypeptide. In some embodiments, the reference is the cell comprising a mutant RBM20 polypeptide contacted with a vehicle control. In some embodiments, the reference is the cell comprising a wild type RBM20 polypeptide contacted with the compound. In some embodiments, the size and/or number of the RBM20 condensates is determined using an imaging technique, such as a fluorescent imaging technique.

In some embodiments, described herein is a method of identifying a compound that decreases the amount of a RBM20 polypeptide in the cytoplasm of a cell, the method comprising: (a) combining the compound and a composition comprising the cell; and (b) obtaining a first measurement of the amount of the RBM20 polypeptide in at least a portion of the cytoplasm of the cell; and (c) comparing the first measurement to a reference, thereby identifying a compound that decreases the amount of the RBM20 polypeptide in the cytoplasm of the cell. In some embodiments, the reference comprises an aliquot of the composition comprising the cell admixed with a control agent. In some embodiments, the reference is a second measurement of the amount of the RBM20 polypeptide in at least a portion of the cytoplasm of the cell, and wherein the second measurement is taken at a different time than the first measurement. In some embodiments, the cell is modified using CRISPR to knockout the endogenous RBM20 gene and is modified to express a heterologous RBM20 polypeptide comprising a fluorescent label. In some embodiments, the RBM20 polypeptide is a mutant RBM20 polypeptide, such as a R636S RBM20 polypeptide. In some embodiments, the RBM20 polypeptide is a wild type polypeptide. In some embodiments, the reference is the cell comprising a mutant RBM20 polypeptide contacted with a vehicle control. In some embodiments, the reference is the cell comprising a wild type RBM20 polypeptide contacted with the compound. In some embodiments, the size and/or number of the RBM20 condensates is determined using an imaging technique, such as a fluorescent imaging technique.

In some embodiments, the cell-based methods described herein are designed to identify a compound, or a portion thereof, that modulates one or more characteristics associated with one or more RBM20 condensates in the cytoplasm of a cell and/or the RBM20 polypeptide in the cytoplasm of a cell, wherein the one or more characteristics are identified as being associated with disease development and/or progression. For example, in some embodiments, it is desirable to dissipate the one or more RBM20 condensates in the cytoplasm of the cell, and thus the one or more characteristics assessed using the methods described herein include one or more of the location of the one or more RBM20 condensates, distribution of the one or more RBM20 condensates and/or the RBM20 polypeptide, number of the one or more RBM20 condensates, size of the one or more RBM20 condensates, ratio of the amount of one or more RBM20 condensates and a reference condensate, a functional activity associated with the one or more RBM20 condensates, composition of the one or more RBM20 condensates, co-localization of the one or more RBM20 condensates with a biomolecule, diffusion coefficient of a component of the one or more RBM20 condensates, stability of the one or more RBM20 condensates, dissolution or reduction in size of the one or more RBM20 condensates, surface area of the one or more RBM20 condensates, sphericity of the one or more RBM20 condensates, liquidity of the one or more RBM20 condensates, and solidification of the one or more RBM20 condensates. In some embodiments, it is desirable to rescue the localization of a components of the RBM20 condensates in the cytoplasm of the cell, such as return a wild type RBM20 polypeptide to the nucleus, or return a non-RBM20 polypeptide back to its normal location (e.g., the location before getting sequestered inside the cytoplasmic RBM20 condensate), and thus the one or more characteristics assessed using the methods described herein include on or more of the functional activity associated with the one or more RBM20 condensates, composition of the one or more RBM20 condensates, co-localization of the one or more RBM20 condensates with a biomolecule, diffusion coefficient of a component of the one or more RBM20 condensates, dissolution or reduction in size of the one or more RBM20 condensates, location of the RBM20 polypeptide, amount of the RBM20 polypeptide or a precursor thereof, condensate partitioning of the RBM20 polypeptide into the one or more RBM20 condensates, functional activity associated with the RBM20 polypeptide, aggregation of the RBM20 polypeptide, post-translational modification status of the RBM20 polypeptide, and amount of a RBM20 polypeptide degradation product.

In some embodiments, the cell-based methods described herein comprise establishing a cell line that expresses (transient, constitutive, or stable cell line with inducible expression) a RBM20 polypeptide (wild type or mutant). For example, a plasmid encoding the RBM20 polypeptide (wild type or mutant) can be transiently transfected (e.g., by electroporation) into a cell (e.g., H9C2), allowing RBM20 polypeptide to express to the desired amount, and/or to the desired phenotype (e.g., without >90% cells forming mutant RBM20 condensates in the cytoplasm), such as about 16-18 hours post-transfection. In some embodiments, a stable cell line carrying a TetOn controlled construct encoding the RBM20 polypeptide (wild type or mutant) can be induced with doxycycline, allowing RBM20 polypeptide to express to the desired amount, and/or to the desired phenotype (e.g., without >90% cells forming mutant RBM20 condensates in the cytoplasm), such as about 24 hours post-induction. Cells can then be placed into plates (e.g., 96-well, or 384-well), e.g., for live cell imaging 24 hours post-transfection or post-induction. In some embodiments, cells are stained with a nuclear dye to indicate cell viability. Test compounds can be admixed with the cells at the same time, then desired markers (e.g., fluorescent labeling of the RBM20 polypeptide, other biomolecule, or the compound, cell morphology, cell proliferation or cytotoxicity) can either be monitored over a period of time (e.g., 1-3 days after applying the compound), or analyzed at the end of the assay. In some embodiments, such methods comprise fixing the cells, staining (e.g., IF staining or DAPI) with the desired markers, and/or analyzing with microscopy. In some embodiments, the methods comprise comparing the characteristics associated with one or more RBM20 condensates and/or the RBM20 polypeptide between cell samples treated and not treated with the test compound. In some embodiments, the methods comprise comparing the characteristics associated with one or more RBM20 condensates and/or the RBM20 polypeptide between a cell line expressing a wild type RBM20 polypeptide and a cell line expressing a mutant RBM20 polypeptide. In some embodiments, non-transduced or non-induced cells serve as controls.

### B. Biochemical Methods

In some aspects, the method disclosed herein is a biochemical method. One of ordinary skill in the art will readily recognize polypeptides, such as a RBM20 polypeptide, and condensates, such as a RBM20 condensate, are dynamic. The methods described herein thus encompass contacting a system with a compound at any point in the life cycle of a RBM20 polypeptide and/or a RBM20 condensates. For example, the methods encompass contacting a system with a compound when a RBM20 polypeptide is in any quantity or has any post-translation modification status, such as the presence, absence, or level of a phosphorylated residue. In some aspects, the methods may also encompass, e.g., contacting a system with a compound when a RBM20 condensates is present in any quantity, including being absent, is undergoing a morphological change, such as a change in size or liquidity, or is changing in composition. In some embodiments, the compound is contacted with the system prior to formation of one or more RBM20 condensates. In some embodiments, the compound is contacted with the system after formation of one or more RBM20 condensates. In some embodiments, the presence, absence, or amount of one or more RBM20 condensates is modulated after the system is contacted with the compound, e.g., one or more RBM20 condensates are present in the system prior to admixing the compound and the amount of the one or more RBM20 condensates increases or decreases after the compound is admixed with the system. In some embodiments of the methods described herein, the system is contacted with the compound more than once, e.g., more than one aliquot of the compound is admixed with the system at more than one point in time.

In some embodiments, described herein is a method of identifying a compound that modulates a characteristic associated with one or more condensates comprising a RBM20 polypeptide (“RBM20 condensates”), the method comprising: (a) admixing the compound and a solution comprising the one or more RBM20 condensates and an extra-condensate solution; and (b) determining the characteristic associated with the one or more RBM20 condensates, wherein a modulation in the characteristic, as compared to a reference, indicates that the compound modulates the characteristic associated with the one or more RBM20 condensates. In some embodiments, the extra-condensate solution comprises a RBM20 polypeptide. In some embodiments, the method further comprises identifying a compound that modulates a characteristic associated with the RBM20 polypeptide. In some embodiments, the RBM20 polypeptide is a wild type RBM20 polypeptide. In some embodiments, the RBM20 polypeptide is a mutant RBM20 polypeptide. In some embodiments, the RBM20 polypeptide comprises a detectable label, such as a fluorescent label.

In some embodiments, described herein is a method of identifying a compound that modulates a characteristic associated with one or more condensates comprising a RBM20 polypeptide (“RBM20 condensates”) and/or the RBM20 polypeptide, the method comprising: (a) combining an agent and a solution comprising the RBM20 polypeptide in the presence of the compound, wherein the agent is capable of causing the formation of the one or more RBM20 condensates and the one or more RBM20 condensates form after the agent contacts the solution; and (b) determining the characteristic associated with the one or more RBM20 condensates and/or the RBM20 polypeptide, wherein a modulation in the characteristic, as compared to a reference, indicates that the compound modulates the characteristic associated with the one or more RBM20 condensates and/or the RBM20 polypeptide. In some embodiments, the agent and compound are admixed prior to combining the agent and the solution comprising the RBM20 polypeptide. In some embodiments, the agent is a vehicle or a buffer, and the agent modulates, such as increases or decreases, the ionic strength of the solution comprising the RBM20 polypeptide. In some embodiments, the agent is a nucleic acid, such as a RNA. In some embodiments, the agent is a molecular crowding agent, such as PEG, dextran, Ficoll, or a combination thereof. In some embodiments, prior to combining the agent and the solution comprising the RBM20 polypeptide, the solution further comprises one or more RBM20 condensates. In some embodiments, the RBM20 polypeptide is a wild type RBM20 polypeptide. In some embodiments, the RBM20 polypeptide is a mutant RBM20 polypeptide. In some embodiments, the RBM20 polypeptide comprises a detectable label, such as a fluorescent label.

Methods of forming condensates are known and can vary based on, e.g., the composition of the condensate. For example, in some embodiments, the condensate is formed by altering, adding, or removing one or more of the temperature of the system, the salt content of the system, the concentration of a component of the condensate, such as a scaffold polypeptide, a nucleic acid, or a RBM20 polypeptide, a buffer in the system, the ionic strength of the system, the pH, or a crowding agent, such as PEG or dextran. Some exemplary methods of forming condensates are also disclosed in Alberti et al., J Mol Biol, 430, 2018, which is herein incorporated by reference in its entirety.

i. Characteristics Associated with One or More RBM20 Condensates and/or the RBM20 Polypeptide in a Biochemical Method

In some aspects, described herein are methods of identifying a compound that modulates a characteristic (including one or more, such as 1, 2, 3, 4, or 5 characteristics), associated with one or more condensates comprising a RBM20 polypeptide (“RBM20 condensates”) and/or the RBM20 polypeptide in an assay system comprising one or more RBM20 condensates and/or a RBM20 polypeptide, such as a solution comprising the one or more RBM20 condensate and/or a RBM20 polypeptide. In some embodiments, the method identifies a compound that modulates a characteristic associated with one or more RBM20 condensates. In some embodiments, the method identifies a compound that modulates a characteristic associated with a RBM20 polypeptide.

In some embodiments, the characteristic associated with the one or more RBM20 condensates and/or the RBM20 polypeptide is based on any one or more of the following: (i) number of the one or more RBM20 condensates; (ii) composition of the one or more RBM20 condensates; (iii) size of the one or more RBM20 condensates; (iv) stability of the one or more RBM20 condensates; (v) dissolution or reduction in size of the one or more RBM20 condensates; (vi) surface area of the one or more RBM20 condensates; (vii) sphericity of the one or more RBM20 condensates; (viii) liquidity of the one or more RBM20 condensates; (ix) solidification of the one or more RBM20 condensates; (x) amount of the RBM20 polypeptide not in the one or more RBM20 condensates; (xi) partitioning of the RBM20 polypeptide into the one or more RBM20 condensates; and (xii) aggregation of the RBM20 polypeptide. In some embodiments, the characteristic associated with the one or more RBM20 condensates and/or the RBM20 polypeptide is as described in any section herein, such as in the cell-based methods section. In some embodiments, the characteristic associated with the one or more RBM20 condensates and/or the RBM20 polypeptide is further based on functional activity associated with one or more RBM20 condensates and/or the RBM20 polypeptide, such as RNA splicing, or binding ability (e.g., binding affinity) of the RBM20 polypeptide with a biomolecule (e.g., non-RBM20 polypeptide, or nucleic acid) or a compound (or a portion thereof).

In some embodiments, the number of the one or more RBM20 condensates is the total number of RBM20 condensates. In some embodiments, the number of the one or more RBM20 condensates is an estimate of the total number of RBM20 condensates in the system. In some embodiments, the number of the one or more RBM20 condensates is the number of RBM20 condensates in a portion of the system, such as a solution comprising the one or more RBM20 condensates, e.g., a field of view.

In some embodiments, the composition of the one or more RBM20 condensates is the amount of the RBM20 polypeptide relative to at least one other component of the one or more RBM20 condensates. In some embodiments, the composition of the one or more RBM20 condensates is the presence, level, or absence of at least one component other than the RBM20 polypeptide, such as a polypeptide, nucleic acid, or compound. In some embodiments, the composition of the one or more RBM20 condensates is the amount of a wild type RBM20 polypeptide relative to a mutant RBM20 polypeptide. In some embodiments, the composition of the one or more RBM20 condensates is the amount of a first mutant RBM20 polypeptide relative to a second mutant RBM20 polypeptide. In some embodiments, the composition of the one or more RBM20 condensates is the amount of a first RBM20 polypeptide relative to a second RBM20 polypeptide, wherein the first and second RBM20 polypeptide have a composition difference, such as a difference in a post-translation modification.

In some embodiments, the size of the one or more RBM20 condensates is based on the largest condensate-crossing dimension measurement, such as diameter, of each of the one or more RBM20 condensates. In some embodiments, the size of the one or more RBM20 condensates is based on the perimeter of each of the one or more RBM20 condensates. In some embodiments, the size of the one or more RBM20 condensates is based the cross-sectional area of each of the one or more RBM20 condensates, or an imaged representation thereof, such as from a top-down view. In some embodiments, the size of the one or more RBM20 condensates is based on the volume of each of the one or more RBM20 condensates. In some embodiments, the characteristic associated with the one or more RBM20 condensates is based on the average size of the one or more RBM20 condensates. In some embodiments, the characteristic associated with the one or more RBM20 condensates is based on the size distribution (such as d5, d10, d90, or d95) of the one or more RBM20 condensates.

In some embodiments, the characteristic associated with the one or more RBM20 condensates is based on the amount of the one or more RBM20 condensates. In some embodiments, the amount of the one or more RBM20 condensates is based on the number and size of the one or more RBM20 condensates. In some embodiments, the amount of the one or more RBM20 condensates is based on the number and size of the one or more RBM20 condensates in a portion of the cytoplasm. In some embodiments, the number and size of the one or more RBM20 condensates is as described herein.

In some embodiments, the stability of the one or more RBM20 condensates is the stability of the one or more RBM20 condensates over time, in the presence of a stressor, such as a compound that reduces the size of the one or more RBM20 condensates, or in the presence of a compound. In some embodiments, the stability is the maintenance of a size or number of the one or more RBM20 condensates.

In some embodiments, the dissolution or reduction in size of the one or more RBM20 condensates is based on the largest condensate-crossing dimension measurement, such as diameter, of each of the one or more RBM20 condensates. In some embodiments, the dissolution or reduction in size of the one or more RBM20 condensates is based on the perimeter of each of the one or more RBM20 condensates. In some embodiments, the dissolution or reduction in size of the one or more RBM20 condensates is based on the average size of the one or more RBM20 condensates. In some embodiments, the dissolution or reduction in size of the one or more RBM20 condensates is based on the size distribution (such as d5, dl 0, d90, or d95) of the one or more RBM20 condensates.

In some embodiments, the surface area of the one or more RBM20 condensates is an estimated surface area based on the perimeter of each of the one or more RBM20 condensates.

In some embodiments, the sphericity of the one or more RBM20 condensates is based on how closely each of the one or more RBM20 condensates resembles a perfect sphere. In some embodiments, the sphericity of the one or more RBM20 condensates is an estimate sphericity based on a cross-section or top-down view of each of the one or more RBM20 condensates. In some embodiments, characteristic associated with one or more RBM20 condensates is the shape of each of the one or more RBM20 condensates. In some embodiments, the characteristic associated with one or more RBM20 condensates is the portion of the one or more RBM20 condensates having a shape type or meeting a shape parameter.

In some embodiments, the liquidity and/or solidification of the one or more RBM20 condensates is based on how the one or more RBM20 condensates fuse with each other, and/or changes in the structure, size, shape, sphericity, volume, number, and/or or surface area of each of the one or more RBM20 condensates over time. In some embodiments, the liquidity and/or solidification of the one or more RBM20 condensates is based on fiber formation.

In some embodiments, the aggregation of the RBM20 polypeptide is the non-phase separated aggregation of the RBM20 polypeptide.

ii. Determining Characteristics Associated with One or More RBM20 Condensates and/or the RBM20 Polypeptide in a Biochemical Method

The characteristics associated with one or more RBM20 condensates and/or the RBM20 polypeptide in the assay system, such as a solution comprising the one or more RBM20 condensates, in some embodiments, may be determined based on any one or more of, e.g., assessment of the one or more RBM20 condensates and/or the RBM20 polypeptide in the system or a portion of, or assessment of another biomolecule, such as another RBM20 condensate component. In some embodiments, the characteristic associated with the one or more RBM20 condensates and/or the RBM20 polypeptide is determined as described in any section herein, such as in the cell-based methods section.

In some embodiments, determining the characteristic associated with the one or more RBM20 condensates and/or the RBM20 polypeptide is based on an assessment of at least a portion of the assay system. In some embodiments, the portion is a field of view, such as a field of view of a microscope, or a portion thereof. In some embodiments, the portion is a defined area of an image. In some embodiments, the defined area is arbitrarily defined. In some embodiments, determining the characteristic associated with the one or more RBM20 condensates and/or the RBM20 polypeptide is based on replicate assessments. In some embodiments, the replicate assessments are based on more than one portion of an image or more than one image. In some embodiments, determining the characteristic associated with the one or more RBM20 condensates and/or the RBM20 polypeptide is based on an average or distribution obtained from two or more portions of an image, two or more images, or two or more portions obtained from at least two or more images.

In some embodiments, determining a characteristic associated with the one or more RBM20 condensates and/or the RBM20 polypeptide is based on an imaging technique. In some embodiments, the imaging technique provides data to assess the characteristic associated with the one or more RBM20 condensates and/or the RBM20 polypeptide. In some embodiments, the imaging technique comprises obtaining an image of a system or a potion thereof. In some embodiments, the image is a two-dimensional image. In some embodiments, the image is a three-dimensional image or rendering thereof. In some embodiments, the imaging technique is coupled with another method feature useful for the methods described herein, such as a fluorescence activated cell sorter (FACS) technique or FAPS technique.

In some embodiments, the methods described herein comprise imaging a sample or a portion thereof, such as a solution comprising the one or more RBM20 condensates, via an imaging technique. In some embodiments, the imaging technique is a fluorescence imaging technique. In some embodiments, the imaging technique comprises a fluorescence imaging technique. In some embodiments, the imaging technique comprises a colorimetric and a fluorescence imaging technique. In some embodiments, the detected light is due to direct labeling of a target, such incorporation or conjugation of a label into a compound or a RBM20 polypeptide. In some embodiments, the detected light is due to indirect labeling of a target, such a labeled probed that specifically binds to a RBM20 polypeptide, e.g., a labeled anti-RBM20 antibody or fragment thereof.

In some embodiments, the characteristic associated with one or more RBM20 condensates and/or the RBM20 polypeptide is determined over a period of time, e.g., at two or more time points. In some embodiments, determining the characteristic associated with one or more RBM20 condensates and/or the RBM20 polypeptide comprises assessing the change in the characteristic over a period of time.

In some embodiments, the characteristic associated with one or more RBM20 condensates and/or the RBM20 polypeptide is determined using one or more measurements and/or techniques. In some embodiments, more than one characteristic associated with one or more RBM20 condensates and/or the RBM20 polypeptide is determined using one or more measurements and/or techniques.

In some embodiments, the methods described herein comprise techniques for, e.g., visualizing, analyzing, and/or quantifying polypeptide and/or precursors thereof. Such techniques are well known by one of ordinary skill in the art. For example, encompassed herein are microscopy techniques for visualizing polypeptides, such as fluorescently labeled polypeptides. Also encompassed herein are mass spectrometry techniques for analyzing the composition of polypeptides, including post-translation modifications, quantifying polypeptides, and studying the composition of RBM20 condensates. Also encompassed herein are functional assays for assessing cellular processes. Also encompassed herein are enrichment and/or isolation techniques, e.g., centrifuge techniques for isolating polypeptides and/or RBM20 condensates or affinity-based techniques for isolating polypeptides or nucleic acids.

In some embodiments, the number of the one or more RBM20 condensates is determined by assessing the total number of RBM20 condensates in the system, such as the solution comprising the one or more RBM20 condensates. In some embodiments, the number of the one or more RBM20 condensates is determined by assessing the number of RBM20 condensates in a portion, such as a field of view, of the system. In some embodiments, the number of the one or more RBM20 condensates is determined by estimating the total number of RBM20 condensates in the system based on measurements of less than the total of the system.

In some embodiments, the composition of the one or more RBM20 condensates is determined by assessing for the amount of the RBM20 polypeptide in the one or more RBM20 condensates. In some embodiments, the composition of one or more RBM20 condensates is determined by assessing for the presence, absence, or level of at least one other component of or associated with one or more RBM20 condensates. In some embodiments, determining the composition of the one or more RBM20 condensates comprises determining at least one other components of or associated with one or more RBM20 condensates relative to the RBM20 polypeptide. In some embodiments, the other component is assessed, such as measured, directly or indirectly. In some embodiments, the other component is assessed using a mass spectrometry technique, such as APEX or XL-MS. In some embodiments, the other component is assessed using a labeling technique, such as directly conjugating a label to the other component or using an immuno-label. In some embodiments, the one or more RBM20 condensates is isolated and/or enriched.

In some embodiments, the size of the one or more RBM20 condensates is determined by assessing the largest condensate-crossing dimension measurement, such as diameter, of each of the one or more RBM20 condensates. In some embodiments, the size of the one or more RBM20 condensates is determined by assessing the perimeter of each of the one or more RBM20 condensates. In some embodiments, the size of the one or more RBM20 condensates is determined by assessing the cross-sectional area of each of the one or more RBM20 condensates, or an imaged representation thereof, such as from a top-down view. In some embodiments, the size of the one or more RBM20 condensates is determined by a particle size measuring technique, such as a dynamic light scattering technique.

In some embodiments, the stability of the one or more RBM20 condensates is determined based on a fusion or fission technique. In some embodiments, the stability of the one or more RBM20 condensates is determined based on changes in the structure of each of the one or more RBM20 condensates over time, in the presence of a cellular activity, or in the presence of a compound. In some embodiments, the stability is of the one or more RBM20 condensates is determined based on assessing any one or more of the size, shape, sphericity, volume, or surface area of each of the one or more RBM20 condensates.

In some embodiments, the dissolution or reduction in size of the one or more RBM20 condensates is determined based on changes in the structure of each of the one or more RBM20 condensates over time, in the presence of a cellular activity, or in the presence of a compound. In some embodiments, the dissolution or reduction in size of the one or more RBM20 condensates is determined based on assessing any one or more of the size, shape, sphericity, volume, number (including absence thereof), or surface area of each of the one or more RBM20 condensates.

In some embodiments, the surface area of the one or more RBM20 condensates is determined based on estimating a surface area using measured parameters (e.g., perimeter, largest condensate-crossing dimension measurement) of each of the one or more RBM20 condensates.

In some embodiments, the sphericity of the one or more RBM20 condensates is determined based on a 3D lattice light sheet technique. In some embodiments, the sphericity of the one or more RBM20 condensates is determined based on a 2D imaging technique. In some embodiments, the sphericity of the one or more RBM20 condensates is determined based on a cross-section or top-down view of each of the one or more RBM20 condensates.

In some embodiments, the liquidity and/or solidification of one or more RBM20 condensates is determined based on a fusion or fission technique. In some embodiments, the liquidity and/or solidification of one or more RBM20 condensates is determined based on changes in the structure of each of the one or more RBM20 condensates over time. In some embodiments, the liquidity and/or solidification of one or more RBM20 condensates is determined based on assessing changes in any one or more of the size, shape, sphericity, volume, number, or surface area of each of the one or more RBM20 condensates.

In some embodiments, the amount of the RBM20 polypeptide not in the one or more RBM20 condensates is determined based on assessing the amount of the RBM20 polypeptide in the one or more RBM20 condensates. In some embodiments, the amount of the RMB20 polypeptide not in the one or more RBM20 condensates is determined based on assessing the amount of the RBM20 polypeptide in the extra-condensate solution.

In some embodiments, the condensate partitioning of the RBM20 polypeptide into the one or more RBM20 condensates is determined based on the amount of the RBM20 polypeptide out of one or more RBM20 condensates as compared to the amount of the RBM20 polypeptide in or associated with one or more RBM20 condensates.

In some embodiments, the aggregation of the RBM20 polypeptide is determined based on the presence, absence, or level of non-phase separated RBM20 polypeptide aggregation.

In some embodiments, the characteristic associated with one or more RBM20 condensates and/or the RBM20 polypeptide is determined based on a fluorescence recovery after photobleaching (FRAP) technique. In some embodiments, the FRAP technique is performed to assess complete condensates, for example, to measure the exchange of components of the one or more RBM20 condensates with the cytoplasm. In some embodiments, the FRAP technique is performed to assess half condensates, for example, to measure the internal dynamics within the one or more RBM20 condensates.

In some embodiments, the characteristic associated with one or more RBM20 condensates and/or the RBM20 polypeptide is determined based on photo-conversion of a fluorophore, such as Dendra2.

In some embodiments, the characteristic associated with one or more RBM20 condensates and/or the RBM20 polypeptide is determined based on a fluorescence correlation spectroscopy technique.

In some embodiments, the characteristic associated with one or more RBM20 condensates and/or the RBM20 polypeptide is determined based on temperature responsiveness of the one or more RBM20 condensates and/or the RBM20 polypeptide.

In some embodiments, the characteristic associated with one or more RBM20 condensates and/or the RBM20 polypeptide is determined based on tracking condensate fusion and/or fission, such as fusion and/or fission in a cell. In some embodiments, condensate fusion and/or fission is determined based on an optical tweezer technique. In some embodiments, the optical tweezer technique is used to determine the surface tension of RBM20 condensate.

In some embodiments, the characteristic associated with one or more RBM20 condensates and/or the RBM20 polypeptide is determined based on a 3D lattice light sheet technique.

In some embodiments, the characteristic associated with one or more RBM20 condensates and/or the RBM20 polypeptide is determined based on a super resolution imaging technique, such as stimulated emission depletion (STED) microscopy, stochastic optical reconstruction microscopy (STORM), or photoactivated localization microscopy (PALM), or a hybrid thereof.

In some embodiments, characteristic associated with one or more RBM20 condensates and/or the RBM20 polypeptide is determined based on ultraviolet-visible (UV-Vis) spectroscopy, small-angle x-ray scattering, or Static and Dynamic Light Scattering (SLS/DLS) technique. For example, light scattering methods like dynamic light scattering (DLS), static light scattering (STS) and small-angle light scattering (SLS) can be used to determine the size and shape of condensates. Also see various in vitro condensate analysis methods described in Basturea, G. N. (“Biological Condensates,” MATER METHODS 2019; 9:2794).

In some embodiments, the characteristic associated with one or more RBM20 condensates and/or the RBM20 polypeptide is determined in the presence of a stressor. For example, in some embodiments, the stressor is one or more of oxidative stress, ATP depletion or presence, a condensate dissolving compound, temperature, such as heat, or mechanical stress, e.g., such as during irregular or excessive frequency of heart beating.

In some embodiments, the characteristic associated with one or more RBM20 condensates and/or the RBM20 polypeptide is determined in the presence of external mechanical force, such as applied using an atomic force microscopy technique.

In some embodiments, the characteristic associated with one or more RBM20 condensates and/or the RBM20 polypeptide is determined using one or more RBM20 polypeptides expressed and purified from a baculovirus system.

In some embodiments, the characteristic associated with one or more RBM20 condensates and/or the RBM20 polypeptide is determined using reconstitution of the one or more RBM20 condensates in the presence of a molecular crowder, such as PEG or dextran. In some embodiments, the characteristic associated with one or more RBM20 condensates and/or the RBM20 polypeptide is determined using reconstitution of the one or more RBM20 condensates not in the presence of a molecular crowder, such as PEG or dextran. In some embodiments, the characteristic associated with one or more RBM20 condensates and/or the RBM20 polypeptide is determined using reconstitution of the one or more RBM20 condensates in the presence of a biopolymer, such as a RNA, DNA, or actin. In some embodiments, the characteristic associated with one or more RBM20 condensates and/or the RBM20 polypeptide is determined using reconstitution of the one or more RBM20 condensates in the presence of a salt or buffer.

In some embodiments, the characteristic associated with one or more RBM20 condensates and/or the RBM20 polypeptide is determined by obtaining data for a phase diagram, e.g., obtained via changing two variables, such as selected from salt concentration, protein concentration, RNA concentration, DNA concentration, biomolecule concentration, pH, and temperature.

In some embodiments, the characteristic associated with one or more RBM20 condensates and/or the RBM20 polypeptide is determined by assessing the dissolution of one or more RBM20 condensates in response to different buffer conditions, e.g., buffers having different salts and concentrations thereof, pHs, hydrotropes and concentrations thereof, ATP concentrations, nucleotides and concentrations thereof, metabolites and concentrations thereof.

In some embodiments, the modulation in the characteristic is based on a change in the presence, absence, or level of the one or more RBM20 condensates and/or the RBM20 polypeptide. In some embodiments, the modulation in the characteristic is based on a decrease in the number of the one or more RBM20 condensates. In some embodiments, the modulation in the characteristic is based on an increase in the number of the one or more RBM20 condensates. In some embodiments, the modulation in the characteristic is based on the formation of the one or more RBM20 condensates, such as the formation of the one or more RBM20. In some embodiments, the modulation in the characteristic is based on a decrease in the size of the one or more RBM20 condensates. In some embodiments, the modulation in the characteristic is based on an increase in the size of the one or more RBM20 condensates.

In some embodiments, the methods described herein comprises determining the specificity of a compound for modulating a characteristic associated with one or more RBM20 condensates and/or the RBM20 polypeptide. For example, in some embodiments, the method comprises determining modulation of a characteristic associated with a first RBM20 condensates and a second RBM20 condensate, wherein the compound modulates the characteristic in the first RBM20 condensate and not the second RBM20 condensate. In some embodiments, the first RBM20 condensate comprises a wild type RBM20 polypeptide and the second RBM20 condensate comprises a mutant RBM20 polypeptide. In some embodiments, the first RBM20 condensate comprises a first mutant RBM20 polypeptide and the second RBM20 condensate comprises a second mutant RBM20 polypeptide, wherein the first and second mutant RBM20 polypeptides are different.

In some embodiments, the method comprises determining modulation of a characteristic associated with a first RBM20 condensates and a second RBM20 condensate, wherein the compound modulates the characteristic in the first RBM20 condensate and the second RBM20 condensate. In some embodiments, the first RBM20 condensate comprises a wild type RBM20 polypeptide and the second RBM20 condensate comprises a mutant RBM20 polypeptide. In some embodiments, the first RBM20 condensate comprises a first mutant RBM20 polypeptide and the second RBM20 condensate comprises a second mutant RBM20 polypeptide, wherein the first and second mutant RBM20 polypeptides are different.

iii. Biochemical References

In some embodiments, the methods described herein determine a modulation in a characteristic associated with one or more RBM20 condensates and/or the RBM20 polypeptide as compared to a reference.

In some embodiments, the reference is an established value for a characteristic. In some embodiments, the reference is a system, such as a solution comprising the one or more RBM20 condensates, contacted with a vehicle control. In some embodiments, the reference is a system, such as a solution comprising the one or more RBM20 condensates, contacted with a reference compound, such as a negative or positive control reference compound. In some embodiments, the reference is a system, such as a solution comprising the one or more RBM20 condensates, contacted with the compound, wherein for the reference the characteristic associated with one or more RBM20 condensates and/or the RBM20 polypeptide is determined at a different time. In some embodiments, the reference is a system, such as a solution comprising the one or more RBM20 condensates, comprising a different RBM20 polypeptide, such as a wild type or mutant RBM20 polypeptide. In some embodiments, the reference is a system, such as a solution comprising the one or more RBM20 condensates, comprising a different level of a RBM20 polypeptide. In some embodiments, the reference is a system, such as a solution comprising the one or more RBM20 condensates, comprising a RBM20 polypeptide having a different post-translation modification status.

### C. RBM20 Polypeptides

In some aspects of the disclosure, described herein are (RNA-binding protein 20) RBM20 polypeptides. In some embodiments, the RBM20 polypeptide is a full-length RBM20 polypeptide. In some embodiments, the RBM20 polypeptide is a modified RBM20 polypeptide, such as a portion of a full-length RBM20 polypeptide.

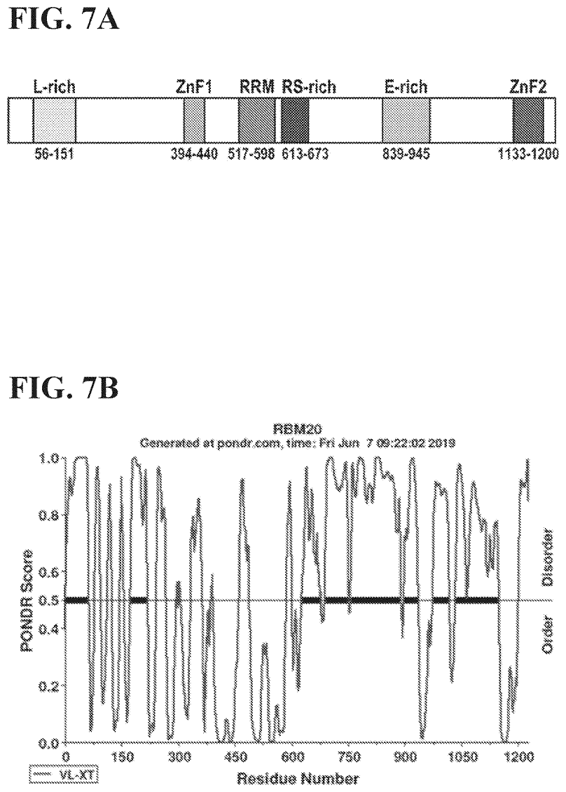

An exemplified amino acid sequence of a RBM20 polypeptide, namely human RBM20 (Uniprot entry Q5T481), is provided below SEQ ID NO:1. As shown in SEQ ID NO:1, an asterisk (*) above a residue denotes residue R636, italic residues denote the RS-region spanning I613-R673, double underline denotes exemplary disordered regions (regions include residues spanning V2-N64, P174-G220, Y628-Q937, and E975-K1150), and bold and italic residue denotes the RSRSP-region spanning R634-P638. FIG. 7A is a schematic illustrating select regions of the RBM20 polypeptide. FIG. 7B shows the results of an analysis of the RBM20 polypeptide sequence for ordered and disordered regions.

(SEQ ID NO: 1)
MVLAAAMSQD ADPSGPEQPD RVACSVPGAR ASPAPSGPRG
MQQPPPPPQP PPPPQAGLPQ IIQNAAKLLD KNPFSVSNPN
PLLPSPASLQ LAQLQAQLTL HRLKLAQTAV TNNTAAATVL
NQVLSKVAMS QPLFNQLRHP SVITGPHGHA GVPQHAAAIP
STRFPSNAIA FSPPSQTRGP GPSMNLPNQP PSAMVMHPFT
GVMPQTPGQP AVILGIGKTG PAPATAGFYE YGKASSGQTY
GPETDGQPGF LPSSASTSGS VTYEGHYSHT GQDGQAAFSK
DFYGPNSQGS HVASGFPAEQ AGGLKSEVGP LLQGTNSQWE
SPHGFSGQSK PDLTAGPMWP PPHNQPYELY DPEEPTSDRT
PPSFGGRLNN SKQGFIGAGR RAKEDQALLS VRPLQAHELN
DFHGVAPLHL PHICSICDKK VFDLKDWELH VKGKLHAQKC
LVFSENAGIR CILGSAEGTL CASPNSTAVY NPAGNEDYAS
NLGTSYVPIP ARSFTQSSPT FPLASVGTTF AQRKGAGRVV
HICNLPEGSC TENDVINLGL PFGKVTNYIL MKSTNQAFLE
MAYTEAAQAM VQYYQEKSAV INGEKLLIRM SKRYKELQLK
KPGKAVAAII QDIHSQRERD MFREADRYGP ERP [symbol] VS
RSLSPRSHTP SFTSCSSSHS PPGPSRADWG NGRDSWEHSP
YARREEERDP APWRDNGDDK RDRMDPWAHD RKHHPRQLDK
AELDERPEGG RPHREKYPRS GSPNLPHSVS SYKSREDGYY
RKEPKAKWDK YLKQQQDAPG RSRRKDEARL RESRHPHPDD
SGKEDGLGPK VTRAPEGAKA KQNEKNKTKR TDRDQEGADD
RKENTMAENE AGKEEQEGME ESPQSVGRQE KEAEFSDPEN
TRTKKEQDWE SESEAEGESW YPTNMEELVT VDEVGEEEDF
IVEPDIPELE EIVPIDQKDK ICPETCLCVT TTLDLDLAQD
FPKEGVKAVG NGAAEISLKS PRELPSASTS CPSDMDVEMP
GLNLDAERKP AESETGLSLE DSDCYEKEAK GVESSDVHPA
PTVQQMSSPK PAEERARQPS PFVDDCKTRG TPEDGACEGS
PLEEKASPPI ETDLQNQACQ EVLTPENSRY VEMKSLEVRS
PEYTEVELKQ PLSLPSWEPE DVFSELSIPL GVEFVVPRTG
FYCKLCGLFY TSEETAKMSH CRSAVHYRNL QKYLSQLAEE
GLKETEGADS PRPEDSGIVP RFERKKL

In some embodiments, the RBM20 polypeptide is a mammalian RBM20 polypeptide. In some embodiments, the RBM20 polypeptide is a primate RBM20 polypeptide. In some embodiments, the RBM20 polypeptide is a human RBM20 polypeptide. In some embodiments, the RBM20 polypeptide is a rat RBM20 polypeptide. In some embodiments, the RBM20 polypeptide is a mouse RBM20 polypeptide.

In some embodiments, the RBM20 polypeptide is a wild type RBM20 polypeptide. In some embodiments, the RBM20 polypeptide is a mutant RBM20 polypeptide. In some embodiments, the mutant RBM20 polypeptide comprises one or more of a substitution, addition, or deletion of one or more amino acid residues. In some embodiments, the mutant RBM20 polypeptide comprises a deletion of one or more of leucine-rich domain, glutamic acid-rich domain, zinc-finger(s), RPM domain, and SR-rich domain. In some embodiments, the mutant RBM20 polypeptide comprises a familial mutation associated with a disease or disorder (e.g., DCM, or sudden cardiac arrest).

In some embodiments, the mutant RBM20 polypeptide comprises a mutation in an intrinsically disorder region (IDR). In some embodiments, the mutant RBM20 polypeptide comprises a mutation in an RS-rich region. In some embodiments, the mutant RBM20 polypeptide comprises a mutation in one or more of the following positions: arginine 634, serine 635, arginine 636, serine 637, and proline 638. In some embodiments, the mutant RBM20 polypeptide comprises a mutation in the arginine 634 position. In some embodiments, the mutant RBM20 polypeptide has a mutation in the serine 635 position. In some embodiments, the mutant RBM20 polypeptide has a mutation in the arginine 636 position. In some embodiments, the mutant RBM20 polypeptide has a mutation in the serine 637 position. In some embodiments, the mutant RBM20 polypeptide has a mutation in the proline 638 position.

In some embodiments, the mutant RBM20 polypeptide comprises one or more of the following mutations: R636S, R636C, R636H, R634Q, 5637G, P638L, S635A, S635E, and S637E. In some embodiments, the mutant RBM20 polypeptide has a R636S mutation. In some embodiments, the mutant RBM20 polypeptide has a R636C mutation. In some embodiments, the mutant RBM20 polypeptide has a R636H mutation. In some embodiments, the mutant RBM20 polypeptide has a R634Q mutation. In some embodiments, the mutant RBM20 polypeptide has a S637G mutation. In some embodiments, the mutant RBM20 polypeptide has a P638L mutation. In some embodiments, the mutant RBM20 polypeptide has a S635A mutation. In some embodiments, the mutant RBM20 polypeptide has a S635E mutation. In some embodiments, the mutant RBM20 polypeptide has a S637E mutation. In some embodiments, the mutant RBM20 polypeptide has S635E, R636S, and S637E mutations.

In some embodiments, the mutant RBM20 polypeptide comprises a mutation outside of an RS-rich region. In some embodiments, the mutant RBM20 polypeptide comprises a mutation between an RS-rich region and an E-rich region. In some embodiments, the mutant RBM20 polypeptide has a mutation in the arginine 716 position. In some embodiments, the mutant RBM20 polypeptide comprises a mutation in an RPM domain. In some embodiments, the mutant RBM20 polypeptide has a mutation in the valine 535 position. In some embodiments, the mutant RBM20 polypeptide comprises a mutation in an E-rich domain. In some embodiments, the mutant RBM20 polypeptide has a mutation in the glutamic acid 913 position. In some embodiments, the mutant RBM20 polypeptide comprises one or more of the following mutations: E913K, R716Q, and V535L. In some embodiments, the mutant RBM20 polypeptide has an E913K mutation. In some embodiments, the mutant RBM20 polypeptide has an R716Q mutation. In some embodiments, the mutant RBM20 polypeptide has a V535L mutation.

In some embodiments, the RBM20 polypeptide, such as the wild type RBM20 polypeptide or the mutant polypeptide, is a modified RBM20 polypeptide, such as a labeled RBM20 polypeptide, a portion of a full-length RBM20 polypeptide, or a RBM20 polypeptide derivative. In some embodiments, the RBM20 polypeptide, is a derivative or an analog. In some embodiments, the RBM20 polypeptide is labeled. In some embodiments, the RBM20 polypeptide is linked, such as covalently, to a label. In some embodiments, the label is a detectable label. In some embodiments, the RBM20 polypeptide comprises a fluorescent label. In some embodiments, the RBM20 polypeptide comprises a fluorescent protein derived from an octocorallia, e.g., Dendronephthya sp, such as Dendra2.

In some embodiments, the RBM20 polypeptide is heterologously expressed in the cell. In some embodiments, the RBM20 polypeptide is homologously expressed in the cell.

In some embodiments, wherein two or more different RBM20 polypeptides are used, such as a cell that expresses a wild type RBM20 polypeptide and a mutant RBM20 polypeptide or a cell that expresses two different mutant RBM20 polypeptides, the two or more different RBM20 polypeptides may be labeled with agents that can be simultaneously be distinguished.

### D. Compounds

In some aspects of the disclosure, described herein are compounds tested and identified using the methods disclosed herein. The compounds encompassed by the description of the present disclosure include, but are not limited to, compounds that are suitable for administration to an individual for a therapeutic or preventative purpose, or a precursor thereof. In some embodiments, the compound is a regulatory approved compound, such as a compound approved for medical treatment by the United States Food and Drug Administration. In some embodiments, the compound is a novel compound. In some embodiments, the compound has a molecular weight of less than 1,000 Da, such as 500 Da or less. In some embodiments, the compound satisfies Lipinski's rule of five. In some embodiments, the compound is a small molecule (such as a therapeutic small molecule that is 1,000 Da or less and/or satisfies Lipinski's rule of five).

In some embodiments, the compound comprises one or more of a small molecule, a polypeptide, a lipid, or a nucleic acid, or a component thereof. In some embodiments, the compound is a small molecule, wherein the compound has a molecular weight of less than or about 900 daltons. In some embodiments, the compound, or a portion thereof, is charged. In some embodiments, the compound, or a portion thereof, is hydrophobic. In some embodiments, the compound, or a portion thereof, is hydrophilic. In some embodiments, the compound comprises an antibody. In some embodiments, the compound comprises a nucleic acid, or a component thereof. In some embodiments, the compound comprises RNA, such as a siRNA, miRNA, mRNA, or lnRNA. In some embodiments, the compound comprises a siRNA, miRNA, or mRNA. In some embodiments, the compound is a non-naturally occurring compound.

In some embodiments, the compound is a precursor or a prodrug. In some embodiments, the compound is metabolized within the composition comprising the cell. In some embodiments, the metabolite of the compound is the active entity that modulates a characteristic associated with one or more “RBM20 condensates” and/or the RBM20 polypeptide.

In some embodiments, the compound comprises a label. In some embodiments, the label is a radioactive label, a colorimetric label, a luminescent label, or a fluorescent label. In some embodiments, the compound is a small molecule comprising a label. In some embodiments, the compound is a small molecule comprising a fluorophore. In some embodiments, the compound is a polypeptide comprising a label. In some embodiments, the compound is a polypeptide comprising a fluorophore. In some embodiments, the compound is a nucleic acid comprising a label. In some embodiments, the compound is a nucleic acid comprising a fluorophore. In some embodiments, the compound is conjugated to the compound covalently or non-covalently.

In some embodiments, wherein a labeled RBM20 polypeptide is used, the compound comprises a label that can be simultaneously distinguished from the label of the labeled RBM20 polypeptide.

In some embodiments of the methods described herein, the compound is present in a vehicle. In some embodiments, the vehicle comprises another agent that is capable of causing the formation of a condensate, such as a nucleic acid, e.g., RNA, or a molecular crowding agent, e.g., REG, dextran, or Ficoll. In some embodiments, the vehicle is such that admixing with a composition comprising a cell or a solution comprising the one or more RBM20 condensates and an extra-condensate solution as described herein results in the formation of one or more RBM20 condensates.

In some embodiments, the compound directly interacts or associates with a RBM20 polypeptide. In some embodiments, the compound indirectly interacts or associates with a RBM20 polypeptide. In some embodiments, the compound interacts or associates with a RBM20 condensate. In some embodiments, the compound interacts or associates with a component of a RBM20 condensate other than a RBM20 polypeptide. In some embodiments, the compound interacts or associates with a biomolecule, wherein the biomolecule interacts or associates with a component of a RBM20 condensate.

### E. Additional Uses of the Methods Described Herein and Further Method Steps

In some aspects, the methods described herein may be used in various formats, for various purposes, and with additional method steps.

In some aspects, the method described herein is used in a screen to assay a library of compounds. In some aspects, the method described herein is used in a screen to assay a library of compounds, wherein the screen comprises a cell-based method. In some aspects, the method described herein is used in a screen to assay a library of compounds, wherein the screen uses cells having a single RBM20 expression profile, e.g., cells all expressing a mutant RBM20 polypeptide and/or cells expressing a substantially similar level of a RBM20 polypeptide. In some aspects, the method described herein is used in a screen to assay a library of compounds, wherein the screen comprises a biochemical method. In some aspects, the method described herein is used in a screen to assay a library of cells. In some aspects, the method described herein is used in a screen to assay a library of cells, wherein the library of cells comprises cell having different RBM20 expression profiles, e.g., different RBM20 mutations, different combinations of RBM20 mutations, and combinations of RBM20 mutations and wild type RBM20. In some embodiments, the methods described herein comprise assessing two or more compounds in a single system, such as a composition comprising a cell.

In some aspects, the method described herein is formatted for any level of throughput, such as high throughput, medium throughput, or low throughput.

In some embodiments, the method described herein further comprises assessing the identified compound using a second cell-based assay. In some embodiments, the method described herein further comprises assessing the identified compound using an in vitro assay. For example, the method further comprises assessing the in vitro binding affinity of the identified compound with one or more components of the RBM20 condensate in non-condensate status (e.g., the light phase). Binding affinity of the compound, or the portion thereof, for the component of the condensate in a non-condensate status can be measured by any appropriate method known in the art, such as MicroScale Thermophoresis (MST), isothermal titration calorimetry (ITC), surface plasmon resonance (SPR), nuclear magnetic resonance (NMR), fluorescence polarization (FP), or Fluorescence Resonance Energy Transfer (FRET) technique. Also see Vuignier et al. “Drug-protein binding: a critical review of analytical tools” (Anal Bioanal Chem, 2010) and Basturea, G. N. (“Biological Condensates,” MATER METHODS 2019; 9:2794) for exemplary methods.

In some embodiments, the method described herein further comprises determining the amount of a compound in a cell, or portion thereof, or a RBM20 condensate. In some embodiments, determining the amount of the compound comprises quantifiably detecting the compound. In some embodiments, determining the amount of the compound comprises quantifiably detecting a label of the compound. In some embodiments, determining the amount of the compound comprises detecting activity of the compound and calculating the amount of compound needed to cause the amount of activity detected. In some embodiments, the amount of compound is determined by mass spectrometry, liquid chromatography, and/or ultraviolet-visible spectrophotometry. In some embodiments, the amount of compound is determined by fluorescence microscopy. Standard curves may be used to aid in determining the amount of the compound.

In some embodiments, provided herein is a method of identifying a compound useful for treating a RBM20-associated disease, the method comprising identifying a compound according to any one of the methods described herein. In some embodiments, the RBM20-associated disease is a cardiomyopathy. In some embodiments, the cardiomyopathy is dilated cardiomyopathy (DCM).

The methods described herein can be used in intelligent screening and/or design of compounds based on a desired compound activity, and/or a desired characteristic associated with one or more RBM20 condensates and/or the RBM20 polypeptide. In some embodiments, the desired behavior of the compound, or the portion thereof, is based on considerations for modulating disease-associated RBM20 condensate to alleviate one or more causes or symptoms of the disease.

In some aspects, provided herein is a method of screening for a candidate compound among a plurality of test compounds, or a portion thereof, based on identifying the modulation of one or more characteristics for each compound and a RBM20 condensate and/or RBM20 polypeptide, using any of the methods described herein. In some embodiments, the candidate compound, or the portion thereof, is selected based on having desired modulation of at least one characteristic associated with one or more RBM20 condensates and/or the RBM20 polypeptide, such as compared to that of a set of screened compounds, or a portion thereof.

In some embodiments, the desired compound activity is selected from one or more of: (i) preferential association of the test compound (or the portion thereof) and the RBM20 condensate in the cytoplasm as compared to a RBM20 condensate in the nucleus; (ii) preferential partitioning of the test compound (or the portion thereof) into the RBM20 condensate (e.g., in the cytoplasm) as compared to a condensate that does not contain RBM20 polypeptide; (iii) preferential binding of the test compound (or the portion thereof) with the RBM20 polypeptide as compared to non-RBM20 polypeptide; (iv) preferential binding of the test compound (or the portion thereof) with a component of the RBM20 condensate, such as a biomolecule that is not RBM20 polypeptide; and (v) preferential binding of the test compound (or the portion thereof) with a mutant RBM20 polypeptide as compared to a wild type RBM20 polypeptide.

In some embodiments, the desired modulation of one or more characteristics associated with one or more RBM20 condensates and/or the RBM20 polypeptide is selected from one or more of: (i) relocating the one or more RBM20 condensates and/or RBM20 polypeptide from cytoplasm to the nucleus; (ii) reducing the amount of RBM20 condensates in the cytoplasm; (iii) increasing the amount of RBM20 condensates in the nucleus; (iv) reducing the size of the one or more RBM20 condensates in the cytoplasm; (v) increasing the ratio of nuclear condensates comprising the RBM20 polypeptide compared to cytoplasmic condensates comprising the RBM20 polypeptide; (vi) restoring and/or increasing a functional activity associated with the one or more RBM20 condensates and/or the RBM20 polypeptide, such as RNA splicing in the nucleus; (vii) excluding a biomolecule from the one or more RBM20 condensates, such as a biomolecule not usually found to be associated with cytoplasmic RBM20 condensates under healthy or non-stressed condition; (viii) relocating a biomolecule from the one or more RBM20 condensates to where it should have been under healthy or non-stressed condition; (ix) reducing stability of one or more cytoplasmic RBM20 condensates; (x) increasing stability of one or more nuclear RBM20 condensates; (xi) dissolving the one or more RBM20 condensates in the cytoplasm; (xii) reducing the surface area of the one or more RBM20 condensates in the cytoplasm; (xiii) restoring the sphericity of the one or more RBM20 condensates in the nucleus; (xiv) increasing the liquidity of the one or more RBM20 condensates in the nucleus and/or the cytoplasm; (xv) reducing the solidification of the one or more RBM20 condensates in the nucleus and/or the cytoplasm; (xvi) relocating the RBM20 polypeptide from the one or more RBM20 condensates in the cytoplasm to the nucleus; (xvii) reducing the amount of the RBM20 polypeptide or a precursor thereof (e.g., when RBM20 is over-expressed or is over-active); (xviii) increasing the amount of the RBM20 polypeptide or a precursor thereof in the nucleus; (xix) promoting partitioning of the RBM20 polypeptide into the one or more RBM20 condensates in the nucleus (e.g., as compared to those in the cytoplasm); (xx) reducing aggregation of the RBM20 polypeptide in the cytoplasm; (xxi) promoting or canceling post-translational modification of the RBM20 polypeptide in the cytoplasm (e.g., ubiquitination or phosphorylation); and (xxii) increasing the amount of a RBM20 polypeptide degradation product in the cytoplasm.

In some aspects, provided herein is a method of designing a candidate compound having desired modulation of one or more characteristics associated with one or more RBM20 condensates and/or the RBM20 polypeptide described herein. In some embodiments, the designing method comprises incorporation of one or more moieties into a candidate compound, wherein each moiety drives, in whole or in part, a desired modulation of the one or more characteristics. For example, in some embodiments, a candidate compound can be designed having a first moiety that drives reducing size of the one or more RBM20 condensates, and a second moiety that drives relocating the RBM20 polypeptide from the one or more RBM20 condensates in the cytoplasm to the nucleus. In some embodiments, a candidate compound can be designed having a first moiety that drives partitioning in a RBM20 condensate in the cytoplasm and a second moiety that drives another function, such as activating or inhibiting a function of another biomolecule or selectively blocking the partitioning of another biomolecule in the RBM20 condensate. In some embodiments, a candidate compound can be designed having a first moiety that drives reducing stability of the one or more RBM20 condensates in the cytoplasm, and dissolving the one or more RBM20 condensates in the cytoplasm, and a second moiety that drives excluding a biomolecule from the one or more RBM20 condensates in the cytoplasm, wherein the biomolecule does not associate with cytoplasmic RBM20 condensates under healthy or non-stressed condition. In some embodiments, the candidate compound can be designed having a moiety that modulates, such as promotes presence or absence, a post-translational modification of an RBM20 polypeptide. In some embodiments, the designing method comprises substituting, removing, or adding a moiety associated with one or more of desired compound activity and/or modulates one or more characteristics associated with one or more RBM20 condensates and/or the RBM20 polypeptide described herein. In some embodiments, the designing method further comprises repeating the designing and testing steps, until the desired need/trait is achieved. In some embodiments, the designing method comprises synthesizing the candidate compound.

In some aspects, provided herein is a method of designing a candidate compound comprising combining two or more moieties, wherein each moiety is associated with desired modulation of one or more characteristics described herein. In some embodiments, the method of designing comprises attaching a moiety that comprises a desired characteristic identified via the methods described herein at any number of position and/or stereochemical orientations. In some embodiments, the resultant candidate compound comprises the combination of desired compound activity and/or desired modulation of one or more characteristics described herein. In some embodiments, the designing method further comprises repeating the designing and testing steps, until the desired need/trait is achieved. In some embodiments, the designing method comprises synthesizing the candidate compound.

In some embodiments, the methods described herein may be used to develop one or more rule sets based on achieved desired modulation of one or more characteristics described herein. In some embodiments, the one or more rule sets can be used as a basis for the identification and/or design of one or more compounds using an approach comprising modeling, computer and/or calculation-based techniques, e.g., bioinformatic, cheminformatic, and/or artificial intelligence (AI)-based identification of a compound having a desired modulation of one or more characteristics described herein. Also provided are computer software for determining and/or applying the one or more rule sets.

In some aspects, provided herein is a method of identifying a candidate compound for treating a disease or disorder associated with RBM20 condensate activity. In some embodiments, the disease or disorder associated with RBM20 condensate activity refers to a disease or a disorder in which any one or more of the following occurs: 1) one or more RBM20 condensates form in the cytoplasm; 2) one or more RBM20 condensates disappear (e.g., dissolute) in the nucleus; 3) one or more RBM20 condensates or a component thereof distribute to a location where the RBM20 condensate or component thereof would not normally locate during healthy condition (e.g., translocate to cytoplasm under disease condition); 4) RBM20 condensate number increases or decreases (e.g., in nucleus and/or cytoplasm); 5) increase or decrease of the number of RBM20 condensates comprising and/or not comprising a component (e.g., RBM20 polypeptide, or one or more other biomolecules that become components of the RBM20 condensate); 6) one or more RBM20 condensates change size, shape, surface area, and/or sphericity; 7) one or more RBM20 condensates change in condensate composition; 8) one or more RBM20 condensates change in liquidity (or dynamic); 9) one or more RBM20 condensates change in solidification; 10) presence and/or amount change of RBM20 fiber formation; 11) change of partitioning of a RBM20 condensate component into a RBM20 condensate; and 12) aggregation of RBM20 polypeptides. Based on the one or more characteristics under the disease condition (and compare to that under a healthy condition), one can identify/screen for/modify/design a candidate compound having a one or more desired compound activity and/or desired modulation of one or more characteristics described herein, using any of the methods described herein.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### III. COMPOSITIONS OF THE DISCLOSURE

In some aspects, the present disclosure provides compositions, such as kits, as described in various facets of the methods disclosed herein.

In some embodiments, provided herein is a cell comprising a RBM20 polypeptide, such as described throughout this application. For example, in some embodiments, provided is a cell expresses a level of a wild type RBM20 polypeptide. In some embodiments, provided is a cell expresses a level of a mutant RBM20 polypeptide. In some embodiments, provided is a cell does not express an endogenous RBM20 polypeptide, such as a knockout cell. In some embodiments, provided is a cell having a heterologous RBM20 polypeptide. In some embodiments, the RBM20 polypeptide is a labeled RBM20 polypeptide.

In some embodiments, provided herein is a composition comprising a RBM20 polypeptide, such as an enriched or isolated RBM20 polypeptide, as described throughout this application. For example, in some embodiments, the RBM20 polypeptide is a wild type RBM20 polypeptide. In some embodiments, the RBM20 polypeptide is a mutant polypeptide. In some embodiments, the RBM20 polypeptide is a labeled RBM20 polypeptide.

### IV. EXEMPLARY EMBODIMENTS

Embodiment 1. A method of identifying a compound that modulates a characteristic associated with one or more condensates comprising a RBM20 polypeptide (“RBM20 condensates”) in the cytoplasm of a cell and/or the RBM20 polypeptide in the cytoplasm of a cell, the method comprising: (a) admixing the compound and a composition comprising the cell, wherein (i) the cell comprises the one or more RBM20 condensates, and/or (ii) the one or more RBM20 condensates form in the cell after the compound contacts the composition; and (b) determining the characteristic associated with the one or more RBM20 condensates and/or the RBM20 polypeptide, wherein a modulation in the characteristic, as compared to a reference, indicates that the compound modulates the characteristic associated with the one or more RBM20 condensates and/or the RBM20 polypeptide.

Embodiment 2. The method of embodiment 1, wherein the characteristic associated with the one or more RBM20 condensates and/or the RBM20 polypeptide is based on any one or more of the following: (i) location of the one or more RBM20 condensates; (ii) distribution of the one or more RBM20 condensates and/or the RBM20 polypeptide; (iii) number of the one or more RBM20 condensates; (iv) size of the one or more RBM20 condensates; (v) ratio of the amount of one or more RBM20 condensates and a reference condensate; (vi) a functional activity associated with the one or more RBM20 condensates; (vii) composition of the one or more RBM20 condensates; (viii) co-localization of the one or more RBM20 condensates with a biomolecule; (ix) diffusion coefficient of a component of the one or more RBM20 condensates; (x) stability of the one or more RBM20 condensates; (xi) dissolution or reduction in size of the one or more RBM20 condensates; (xii) surface area of the one or more RBM20 condensates; (xiii) sphericity of the one or more RBM20 condensates; (xiv) liquidity of the one or more RBM20 condensates; (xv) solidification of the one or more RBM20 condensates; (xvi) location of the RBM20 polypeptide; (xvii) amount of the RBM20 polypeptide or a precursor thereof; (xviii) condensate partitioning of the RBM20 polypeptide into the one or more RBM20 condensates; (xix) a functional activity associated with the RBM20 polypeptide; (xx) aggregation of the RBM20 polypeptide; (xxi) post-translational modification status of the RBM20 polypeptide; and (xxii) amount of a RBM20 polypeptide degradation product.

Embodiment 3. The method of embodiment 2, wherein the modulation in the characteristic is based on a decrease in the number of the one or more RBM20 condensates in the cytoplasm of the cell.

Embodiment 4. The method of embodiment 2 or 3, wherein the modulation in the characteristic is based on a decrease in the amount of the RBM20 polypeptide or a precursor thereof in the cytoplasm of the cell.

Embodiment 5. The method of any one of embodiments 2-4, wherein the modulation in the characteristic is based on dissolution or reduction in size of the one or more RBM20 condensates in the cytoplasm of the cell.

Embodiment 6. The method of any one of embodiments 2-5, wherein the modulation in the characteristic is based on a decrease in the functional activity associated with the one or more RBM20 condensates and/or the RBM20 polypeptide in the cytoplasm of the cell.

Embodiment 7. The method of embodiment 1, wherein the characteristic associated with the one or more RBM20 condensates and/or the RBM20 polypeptide comprises location of the one or more RBM20 condensates, distribution of the one or more RBM20 condensates and/or the RBM20 polypeptide, number of the one or more RBM20 condensates, size of the one or more RBM20 condensates, and ratio of the amount of one or more RBM20 condensates and a reference condensate.

Embodiment 8. The method of embodiment 1, wherein the characteristic associated with the one or more RBM20 condensates and/or the RBM20 polypeptide comprises composition of the one or more RBM20 condensates, and co-localization of the one or more RBM20 condensates with a biomolecule.

Embodiment 9. The method of embodiment 7 or 8, wherein the characteristic associated with the one or more RBM20 condensates and/or the RBM20 polypeptide further comprises the functional activity associated with the one or more RBM20 condensates.

Embodiment 10. The method of embodiment 1, wherein the characteristic associated with the one or more RBM20 condensates and/or the RBM20 polypeptide comprises stability of the one or more RBM20 condensates, dissolution or reduction in size of the one or more RBM20 condensates, and surface area of the one or more RBM20 condensates.

Embodiment 11. The method of embodiment 1, wherein the characteristic associated with the one or more RBM20 condensates and/or the RBM20 polypeptide comprises sphericity of the one or more RBM20 condensates, liquidity of the one or more RBM20 condensates, and solidification of the one or more RBM20 condensates.

Embodiment 12. The method of embodiment 1, wherein the characteristic associated with the one or more RBM20 condensates and/or the RBM20 polypeptide comprises location of the RBM20 polypeptide, and amount of the RBM20 polypeptide or a precursor thereof.

Embodiment 13. The method embodiment 12, wherein the characteristic associated with the one or more RBM20 condensate and/or the RBM20 polypeptide further comprises post-translational modification status of the RBM20 polypeptide.

Embodiment 14. The method of embodiment 12 or 13, wherein the characteristic associated with the one or more RBM20 condensate and/or the RBM20 polypeptide further comprises the functional activity associated with the RBM20 polypeptide.

Embodiment 15. The method of embodiment 1, wherein the characteristic associated with the one or more RBM20 condensates and/or the RBM20 polypeptide comprises co-localization of the one or more RBM20 condensates with a biomolecule, and diffusion coefficient of a component of the one or more RBM20 condensates.

Embodiment 16. The method of embodiment 1, wherein the characteristic associated with the one or more RBM20 condensates and/or the RBM20 polypeptide comprises stability of the one or more RBM20 condensates, and dissolution or reduction in size of the one or more RBM20 condensates.

Embodiment 17. The method of embodiment 1, wherein the characteristic associated with the one or more RBM20 condensates and/or the RBM20 polypeptide comprises surface area of the one or more RBM20 condensates, sphericity of the one or more RBM20 condensates, liquidity of the one or more RBM20 condensates, and solidification of the one or more RBM20 condensates.

Embodiment 18. The method of any one of embodiments 1-17, wherein the RBM20 polypeptide is a wild type RBM20 polypeptide.

Embodiment 19. The method of any one of embodiments 1-17, wherein the RBM20 polypeptide is a mutant RBM20 polypeptide.

Embodiment 20. The method of embodiment 19, wherein the mutant RBM20 polypeptide comprises a mutation in an intrinsically disorder region (IDR).

Embodiment 21. The method of embodiment 19 or 20, wherein the mutant RBM20 polypeptide comprises a mutation in an RS-rich region.

Embodiment 22. The method of any one of embodiments 19-21, wherein the mutant RBM20 polypeptide comprises a mutation in one or more of the following positions: arginine 634, serine 635, arginine 636, serine 637, and proline 638.

Embodiment 23. The method of embodiment 22, wherein the mutant RBM20 polypeptide comprises one or more of the following mutations: R636S, R636C, R636H, R634Q, S637G, P638L, S635A, S635E, and S637E.

Embodiment 24. The method of embodiment 19, wherein the mutant RBM20 polypeptide comprises one or more of the following mutations: E913K, R716Q, and V535L.

Embodiment 25. The method of any one of embodiments 1-24, wherein the RBM20 polypeptide is heterologously expressed in the cell.

Embodiment 26. The method of any one of embodiments 1-24, wherein the RBM20 polypeptide is homologously expressed in the cell.

Embodiment 27. The method of any one of embodiments 1-26, wherein the cell is a model of a cardiac cell type.

Embodiment 28. The method of any one of embodiments 1-27, wherein the cell is a cardiomyocyte.

Embodiment 29. The method of embodiment 27 or 28, wherein the cell is a Rat H9C2 cell.

Embodiment 30. The method of embodiment 27 or 28, wherein the cell is a human AC-16 cell, a patient-derived cardiomyocyte, a human induced pluripotent stem cell differentiated to a cardiomyocyte, or a stem cell differentiated to a cardiomyocyte.

Embodiment 31. The method of any one of embodiments 1-26, wherein the cell is selected from the group consisting of: a HeLa cell, a U2OS cell, a human embryonic kidney cell, a human induced pluripotent stem cell, and a stem cell.

Embodiment 32. The method of any one of embodiments 1-31, wherein the cell is homozygous for alleles encoding the RBM20 polypeptide.

Embodiment 33. The method of any one of embodiments 1-31, wherein the cell is heterozygous for alleles encoding the RBM20 polypeptide.

Embodiment 34. The method of any one of embodiments 1-33, wherein the reference comprises an aliquot of the composition comprising the cell admixed with a control agent.

Embodiment 35. The method of any one of embodiments 1-34, further comprising imaging at least a portion of the composition or the cell.

Embodiment 36. The method of any one of embodiments 1-35, wherein the method further comprises determining one or more cellular features of the cell.

Embodiment 37. The method of any one of embodiments 1-36, further comprising contacting at least a portion of the composition or the cell with a fixative.

Embodiment 38. The method of any one of embodiments 1-37, further comprising contacting at least a portion of the composition or the cell with a stain.

Embodiment 39. The method of any one of embodiments 1-38, further comprising assessing the identified compound using a second cell-based assay.

Embodiment 40. The method of any one of embodiment 1-39, further comprising assessing the identified compound using an in vitro assay.

Embodiment 41. A method of identifying a compound that reduces the size and/or number of condensates comprising a RBM20 polypeptide (“RBM20 condensates”) in the cytoplasm of a cell, the method comprising: (a) determining the size and/or number of the RBM20 condensates in at least a portion of the cytoplasm of the cell subjected to the compound; and (b) comparing the size and/or number of the RBM20 condensates with a reference, thereby identifying the compound that reduces the size and/or number of the RBM20 condensates in the cytoplasm of the cell.

Embodiment 42. The method of embodiment 41, wherein the compound reduces the number of RBM20 condensates.

Embodiment 43. The method of embodiment 41 or 42, wherein the compound reduces the size of RBM20 condensates.

Embodiment 44. A method of identifying a compound that prevents formation or growth of one or more condensates comprising a RBM20 polypeptide (“RBM20 condensates”) in the cytoplasm of a cell, the method comprising: (a) combining the compound and a composition comprising the cell, wherein (i) the cell comprises the one or more RBM20 condensates, and/or (ii) the one or more RBM20 condensates form after the compound contacts the composition; and (b) obtaining a first measurement of the size and/or number of the one or more RBM20 condensates in at least a portion of the cytoplasm of the cell; and (c) comparing the first measurement to a reference, thereby identifying a compound that prevents formation or growth of the one or more RBM20 condensates in the cytoplasm of the cell.

Embodiment 45. The method of embodiment 44, wherein the reference comprises an aliquot of the composition comprising the cell admixed with a control agent.

Embodiment 46. The method of embodiment 44, wherein the reference is a second measurement of the size and/or number of the one or more RBM20 condensates in at least a portion of the cytoplasm of the cell, and wherein the second measurement is taken at a different time than the first measurement.

Embodiment 47. The method of any one of embodiments 44-46, further comprising subjecting the cell to a condition that promotes formation of the one or more RBM20 condensates.

Embodiment 48. A method of identifying a compound that decreases the amount of a RBM20 polypeptide in the cytoplasm of a cell, the method comprising: (a) combining the compound and a composition comprising the cell; (b) obtaining a first measurement of the amount of the RBM20 polypeptide in at least a portion of the cytoplasm of the cell; and (c) comparing the first measurement to a reference, thereby identifying a compound that decreases the amount of the RBM20 polypeptide in the cytoplasm of the cell.

Embodiment 49. The method of embodiment 48, wherein the reference comprises an aliquot of the composition comprising the cell admixed with a control agent.

Embodiment 50. The method of embodiment 49, wherein the reference is a second measurement of the amount of the RBM20 polypeptide in at least a portion of the cytoplasm of the cell, and wherein the second measurement is taken at a different time than the first measurement.

Embodiment 51. A method of identifying a compound that modulates a characteristic associated with one or more condensates comprising a RBM20 polypeptide (“RBM20 condensates”), the method comprising: (a) admixing the compound and a solution comprising the one or more RBM20 condensates and an extra-condensate solution; and (b) determining the characteristic associated with the one or more RBM20 condensates, wherein a modulation in the characteristic, as compared to a reference, indicates that the compound modulates the characteristic associated with the one or more RBM20 condensates.

Embodiment 52. A method of identifying a compound that modulates a characteristic associated with one or more condensates comprising a RBM20 polypeptide (“RBM20 condensates”) and/or the RBM20 polypeptide, the method comprising: (a) combining an agent and a solution comprising the RBM20 polypeptide in the presence of the compound, wherein the agent is capable of causing the formation of the one or more RBM20 condensates and the one or more RBM20 condensates form after the agent contacts the solution; and (b) determining the characteristic associated with the one or more RBM20 condensates and/or the RBM20 polypeptide, wherein a modulation in the characteristic, as compared to a reference, indicates that the compound modulates the characteristic associated with the one or more RBM20 condensates and/or the RBM20 polypeptide.

Embodiment 53. The method of embodiment 51 or 52, wherein the characteristic associated with the one or more RBM20 condensates and/or the RBM20 polypeptide is based on any one or more of the following: (i) number of the one or more RBM20 condensates; (ii) composition of the one or more RBM20 condensates; (iii) size of the one or more RBM20 condensates; (iv) stability of the one or more RBM20 condensates; (v) dissolution or reduction in size of the one or more RBM20 condensates; (vi) surface area of the one or more RBM20 condensates; (vii) sphericity of the one or more RBM20 condensates; (viii) liquidity of the one or more RBM20 condensates; (ix) solidification of the one or more RBM20 condensates; (x) amount of the RBM20 polypeptide not in the one or more RBM20 condensates; (xi) partitioning of the RBM20 polypeptide into the one or more RBM20 condensates; and (xii) aggregation of the RBM20 polypeptide.

Embodiment 54. A method of identifying a compound that modulates the partitioning of a biomolecule for a condensate comprising a RBM20 polypeptide (“RBM20 condensate”), the method comprising: (a) admixing the compound and a composition comprising a cell, wherein (i) the cell comprises the RBM20 condensate, and/or (ii) the RBM20 condensate forms in the cell after the compound contacts the composition; and (b) determining the partitioning of the biomolecule for the RBM20 condensates.

Embodiment 55. The method of claim 54, wherein the biomolecule is a non-RBM20 polypeptide.

Embodiment 56. The method of claim 54, wherein the biomolecule is a wild type RBM20 polypeptide.

Embodiment 57. The method of any one of claims 54-56, wherein the RBM20 condensate comprising the RBM20 polypeptide comprises a mutant RBM20 polypeptide.

Embodiment 58. A method of identifying a compound useful for treating a RBM20-associated disease, the method comprising identifying a compound according to any one of the methods of embodiments 1-57.

Embodiment 59. The method of embodiment 58, wherein the RBM20-associated disease is a cardiomyopathy.

Embodiment 60. The method of embodiment 59, wherein the cardiomyopathy is dilated cardiomyopathy.

### EXAMPLES

### Example 1

This example demonstrates fluorescence imaging analyses of cells engineered to express a fluorescently labeled wild type RBM20 or a fluorescently labeled RBM20 mutant having a single point mutation. HeLa and U2OS (human bone osteosarcoma epithelial cells) cells were engineered using transient transfection to express a wild type human RBM20 polypeptide linked to a Dendra2 label or a mutant RBM20 linked to a Dendra2 label. Following transfection, cells were incubated to allow for RBM20 polypeptide expression. Fluorescent images of cells, and/or portions of cells, were captured using a DeltaVision wide-field deconvoluted system having a 60× oil objective.

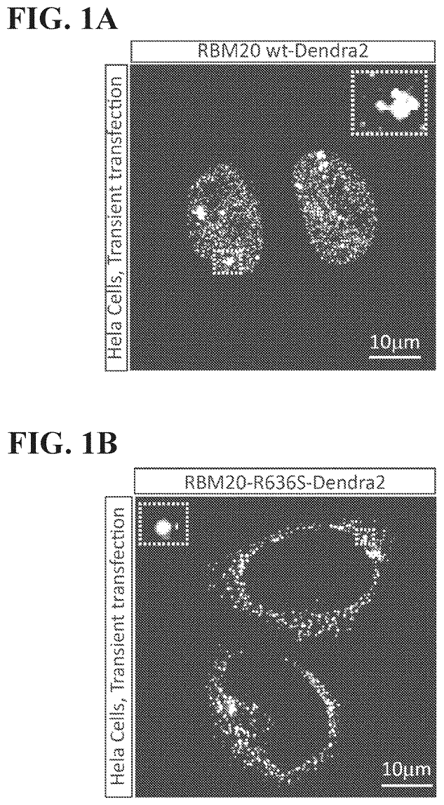

In HeLa cells transfected with a wild type RBM20 polypeptide, RBM20 condensates were observed in the nucleus (FIG. 1A). In HeLa cells transfected with a R636S RBM20 polypeptide, RBM20 condensates were exclusively observed in the cytoplasm (FIG. 1B). Aspects of the images of FIG. 1A and FIG. 1B in the dashed-line boxes correspond to enlarged views featuring condensates.

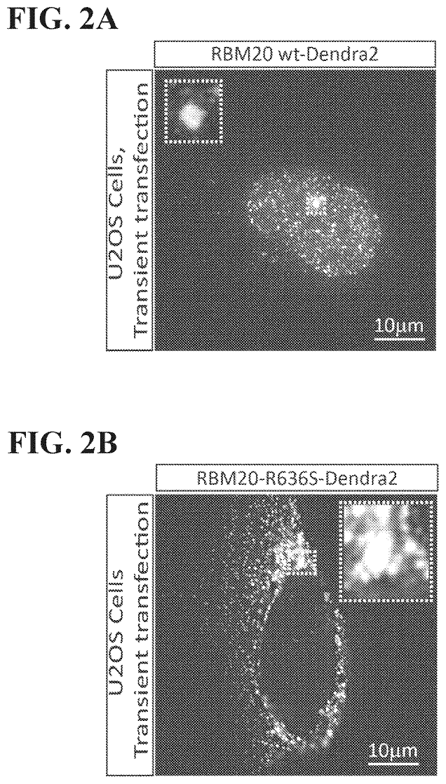

In U2OS cells transfected with a wild type RBM20 polypeptide, RBM20 condensates were observed in the nucleus (FIG. 2A). In U2OS cells transfected with a R636S RBM20 polypeptide, RBM20 condensates were exclusively observed in the cytoplasm (FIG. 2B). Aspects of the images of FIG. 2A and FIG. 2B in the dashed-line boxes correspond to enlarged views featuring condensates.

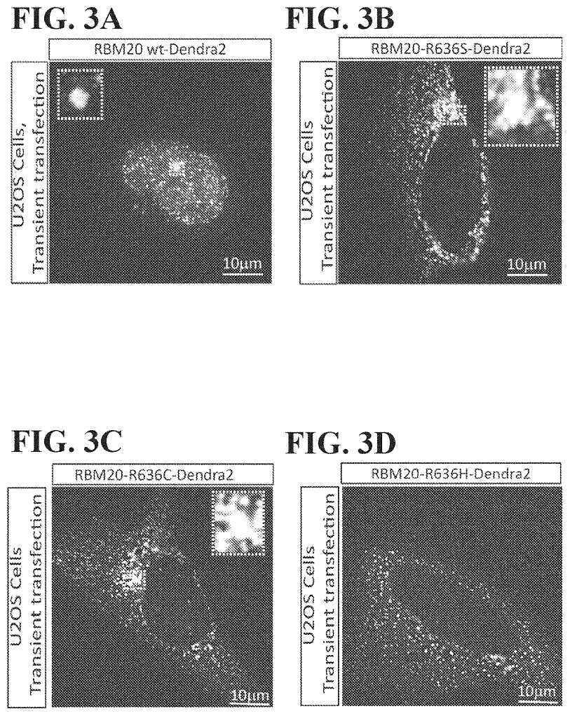

Additional RBM20 polypeptide mutants, namely, R636C and R636H, were expressed and evaluated in U2OS cells as described above. As shown in FIGS. 3A-3D, in U2OS cells transfected with a wild type RBM20 polypeptide, RBM20 condensates were primarily observed in the nucleus (FIG. 3A), and in U2OS cells transfected with a RBM20 mutant polypeptide, RBM20 condensates were exclusively observed in the cytoplasm (FIG. 3B, R636S RBM20; FIG. 3C, R636C RBM20; FIG. 3D, R636H RBM20). Aspects of the images of FIGS. 3A-3C in the dashed-line boxes correspond to enlarged views featuring condensates.

### Example 2

This example demonstrates fluorescence imaging analyses of a cardiomyocyte, namely H9C2 (rat myoblast from embryonic heart), engineered to express a fluorescently labeled wild type RBM20 polypeptide or various fluorescently labeled RBM20 mutant polypeptides having a single point mutation in the RS-rich domain. H9C2 cells were engineered using transient transfection to express a wild type RBM20 polypeptide linked to a Dendra2 label or a mutant RBM20 polypeptide (R636S, R636C, R636H, R634Q, S635A, S637G, P638L) linked to a Dendra2 label. Following transfection, cells were incubated to allow for RBM20 polypeptide expression. Fluorescent images of cells, and/or portions of cells, were captured using a DeltaVision wide-field deconvoluted system having a 60× oil objective.

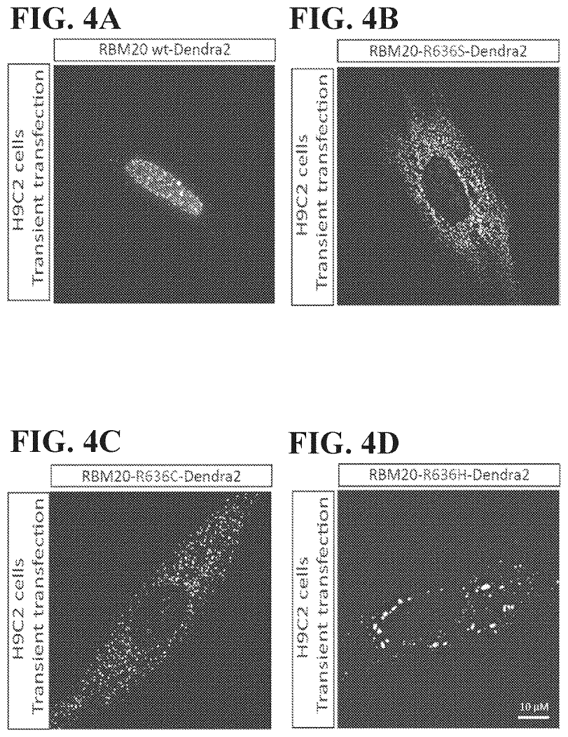

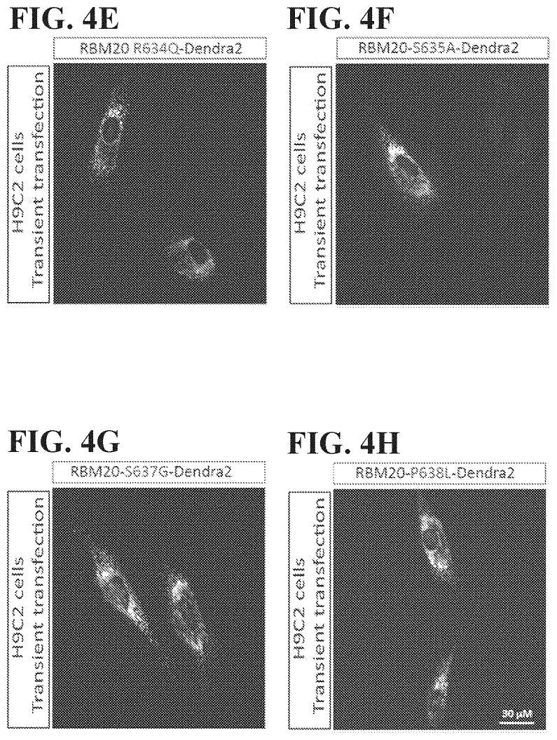

As shown in FIGS. 4A-4H, in H9C2 cells transfected with a wild type RBM20 polypeptide, RBM20 condensates were primarily observed in the nucleus (FIG. 4A), and in H9C2 cells transfected with a RBM20 mutant polypeptide, RBM20 condensates were exclusively observed in the cytoplasm (FIG. 4B, R636S RBM20; FIG. 4C, R636C RBM20; FIG. 4D, R636H RBM20; FIG. 4E, R634Q RBM20; FIG. 4F, S635A RBM20; FIG. 4G, S637G RBM20, FIG. 4H, P638L RBM20).

### Example 3

This example demonstrates fluorescence imaging analyses of various U2OS cells engineered to express fluorescently labeled wild type RBM20. U2OS cells were engineered using transient transfection to express a wild type human RBM20 polypeptide linked to a Dendra2 label. Following transfection, cells were incubated to allow for RBM20 polypeptide expression. Fluorescent images of cells, and/or portions of cells, were captured using a DeltaVision wide-field deconvoluted system having a 60× oil objective.

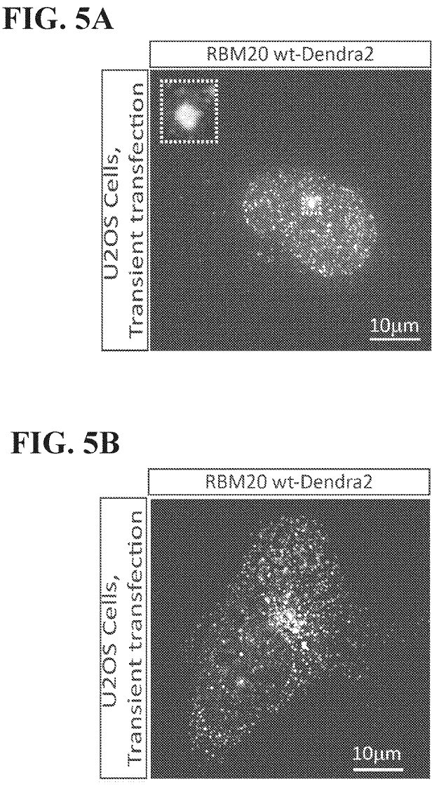

In U2OS cells, it was observed that continued expression of a wild type RBM20 polypeptide can lead to the formation of RBM20 condensates in both the nucleus and cytoplasm. As shown in FIGS. 5A and 5B, some U2OS cells have RBM20 condensates primarily located in the nucleus (FIG. 5A), and some U2OS cells have RBM20 condensates located in both the nucleus and cytoplasm (FIG. 5B). Aspects of the image of FIG. 5A in the dashed-line boxes correspond to enlarged views featuring condensates.

### Example 4

This example demonstrates an assay for identifying a compound that modulates a characteristic associated with one or more RBM20 condensates and/or the RBM20 polypeptide. H9C2 cells were engineered using transient transfection to express a mutant R636S RBM20 polypeptide linked to a Dendra2 label. Following transfection, cells were incubated to allow for RBM20 polypeptide expression. H9C2 cells were admixed with a compound selected from a control (DMSO), lipoamide (30 μM), mitoxantrone (20 μM), or JQ1 (10 μM), and then incubated for 2 hours prior to capturing fluorescent images. Fluorescent images of cells, and/or portions of cells, were captured using a DeltaVision wide-field deconvoluted system having a 60× oil objective.

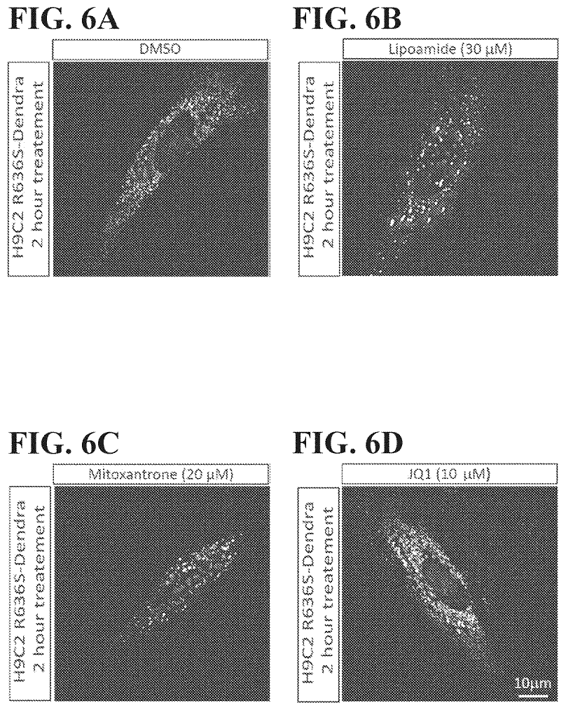

Images of the H9C2 cells treated with a compound and a reference are shown in FIGS. 6A-6D. Using these images, a modulation in a characteristic associated with one or more RBM20 condensates and/or the RBM20 polypeptide, as compared to a reference, can be determined.

### Example 5

This example demonstrates fluorescence imaging analyses of H9C2 cells engineered to express fluorescently labeled wild type RBM20. H9C2 cells were engineered using transient transfection to express a wild type human RBM20 polypeptide linked to a Dendra2 label. Following transfection, cells were incubated to allow for RBM20 polypeptide expression and fluorescent images were captured over a time course. Fluorescent images of cells, and/or portions of cells, were captured using a DeltaVision wide-field deconvoluted system having a 40× oil objective.

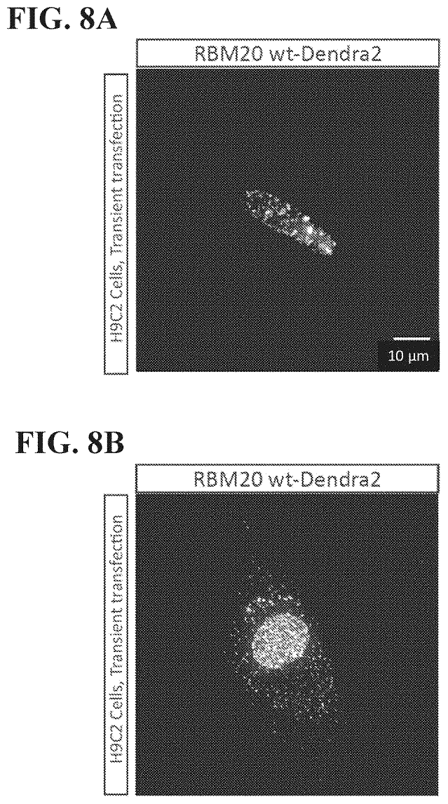

In H9C2 cells, it was observed in earlier time points that expression of a wild type RBM20 polypeptide led to the formation of RBM20 condensates in the nucleus (FIG. 8A). At later time points, it was observed that the amount of RBM20 condensate in the nucleus was increased and continued expression of the RBM20 polypeptide led to the formation of RBM20 condensate in the cytoplasm (FIG. 8B). This time course study demonstrates that increased expression of wild type RBM20 polypeptide can led to saturation in the nucleus, which then leads to the formation of cytoplasmic wild type RBM20 condensates.

### Example 6

This example demonstrates various cell model systems useful for further assessing RBM20 cell systems, including for assessing RBM20 polypeptide localization, condensate formation, and condensate properties.

H9C2 cells were engineered using transient transfection to express either a mutant R636S RBM20 polypeptide linked to a Dendra2 label and a nuclear localization signal (NLS) at the C-terminus, or a mutant R636S RBM20 polypeptide linked to a Dendra2 label without a NLS. Following transfection, cells were incubated to allow for RBM20 polypeptide expression. Cells were then fixed and stained with DAPI to visualize the nucleus. Fluorescent images of cells, and/or portions of cells, were captured using a DeltaVision wide-field deconvoluted system having a 60× oil objective.

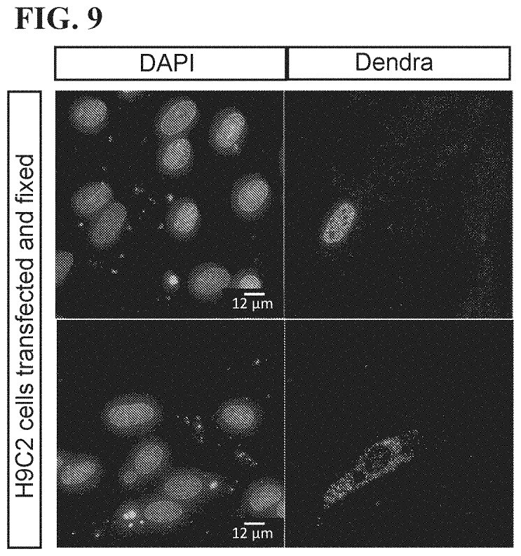

As shown in FIG. 9, adding a NLS to the mutant R636S RBM20 polypeptide (upper panels) rescued nuclear localization of the mutant R636S RBM20 polypeptide as the mutant R636 RBM20 polypeptide without a NLS is exclusively located in the cytoplasm (bottom panels).

H9C2 cells were engineered using transient transfection to express a phospho-mimetic mutant RBM20 polypeptide (R636S, S635E, S637E) linked to a Dendra2 label. As in Example 4, H9C2 cells were also engineered using transient transfection to express a mutant R636S RBM20 polypeptide linked to a Dendra2 label. Following transfection, cells were incubated to allow for RBM20 polypeptide expression. Fluorescent images of cells, and/or portions of cells, were captured using a DeltaVision wide-field deconvoluted system having a 40× oil objective.

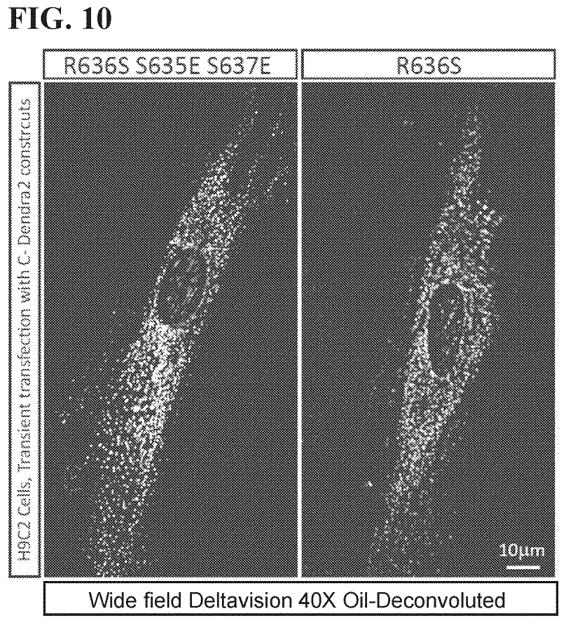

As shown in FIG. 10, the phospho-mimetic mutant (R636S, S635E, S637E) did not rescue the nuclear localization of the mutant R636S RBM20 polypeptide.

H9C2 cells were engineered using transient transfection to express truncated/excised forms of the wild type RBM20 polypeptide linked to a Dendra2 label. Specifically, the forms of RBM20 polypeptide studied were a leucine-rich domain deletion (domain spans amino acids 56-151), a glutamic acid-rich domain deletion (domain spans amino acids 839-945), a zinc finger 1 and zinc finger 2 deletion (zinc finger 1 spans amino acids 394-440; zinc finger 2 spans amino acids 1133-1200), a RNA recognition motif deletion (RRM; domain spans amino acids 517-598), and a serine-arginine rich domain deletion (SR; domain spans amino acids 613-673). Following transfection, cells were incubated to allow for RBM20 polypeptide expression. Fluorescent images of cells, and/or portions of cells, were captured using a DeltaVision wide-field deconvoluted system having a 60× oil objective.

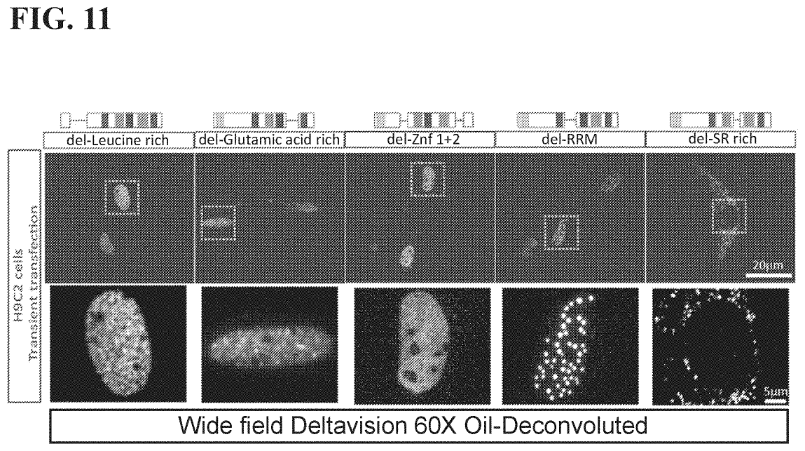

As shown in FIG. 11, deletion of the zinc fingers resulted in increased diffusion of the polypeptide in the nucleus, deletion of the RRM resulted in significantly bigger and more spherical nuclear RBM20 condensate, and deletion of the SR domain resulted in the exclusive formation of cytoplasmic RBM20 condensates. The SR-rich domain deletion findings implicate the role of SR-rich domain in RMB20 nuclear importation. The zinc finger 1 and zinc finger 2 deletion findings implicate the role of zinc fingers in modulating RMB20 nuclear condensates. The RRM deletion findings implicate the role of RNA binding in inhibiting condensation in the nucleus. To further study the RRM deletion model, the fusion of RRM deletion RBM20 condensates was measured and fusion of condensates was observed to occur in less than 50 milliseconds indicating the liquid-like nature of the condensates (data not shown).

### Example 7

The example demonstrates a heterozygous wild type/mutant RBM20 polypeptide cell line and the impact on RBM20 condensate formation and composition.

A first H9C2 cell system was engineered using transient transfection (dual transfection) to express a copy of a mutant R636S RBM20 polypeptide linked to a mCherry label and a copy of a wild type RBM20 polypeptide linked to a Dendra2 label. A second H9C2 cell system was engineered using transient transfection (dual transfection) to express a copy of a mutant R636S RBM20 polypeptide linked to a mCherry label and a copy of a Nuclear Export Signal sequence of HIV-1 (NES; nucleic acid sequence, ctgccccccctggagcgcctgaccctg (SEQ ID NO:2); amino acid sequence, LPPLERLTL (SEQ ID NO:3)). polypeptide linked to a Dendra2 label, serving as control. A third H9C2 cell system was engineered using transient transfection to express a copy of a mutant R636S RBM20 polypeptide linked to a mCherry label. A fourth H9C2 cell system was engineered using transient transfection to express a copy of a wild type RBM20 polypeptide linked to a Dendra2 label. The single transfection cell systems served as mutant and wild type RBM20 polypeptide localization controls, as well as channel bleaching controls. Following transfection, cells were incubated to allow for RBM20 polypeptide expression. Fluorescent images of cells, and/or portions of cells, were captured using a DeltaVision wide-field deconvoluted system having a 40× oil objective.

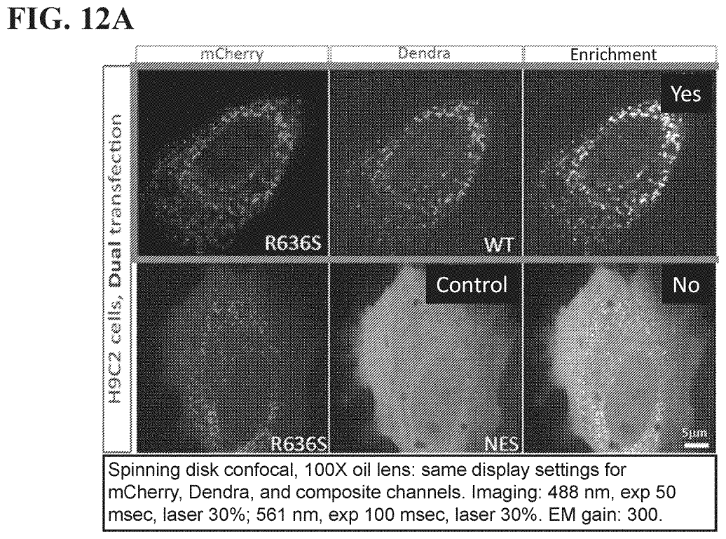

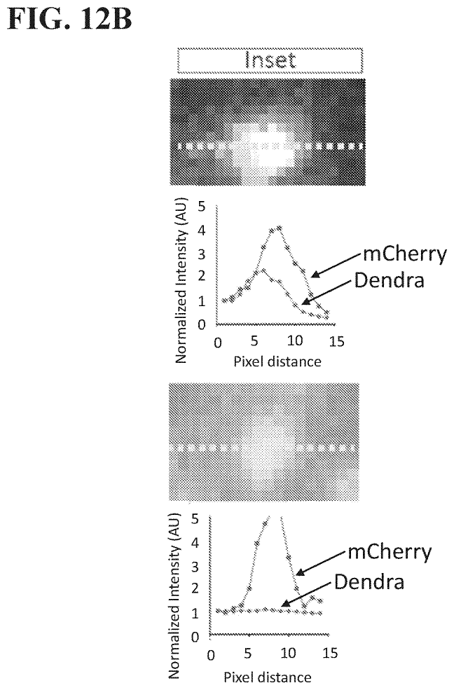

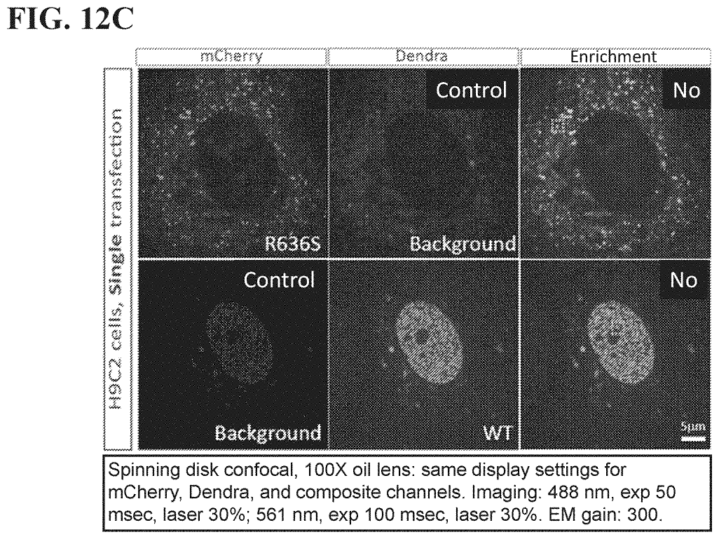

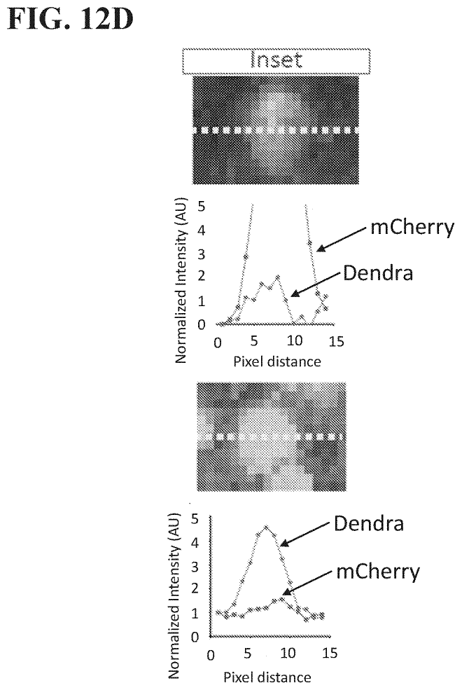

As shown in FIGS. 12A-12D, heterozygous RBM20 polypeptide H9C2 cells were shown to form cytoplasmic RMB20 condensate containing both mutant and wild type RBM20 polypeptides, thus demonstrating that the presence of mutant RBM20 polypeptide can lead to mislocalization of wild type RBM20 polypeptide. As a control, mutant R636S RBM20 polypeptide did not sequester NES into cytoplasmic RMB20 condensate (FIG. 12A bottom panels). Insets are provided in FIG. 12B for the images in FIG. 12A, and insets are provided in FIG. 12D for the images in FIG. 12C.

### Example 8

This example demonstrates fluorescence imaging analyses of cardiomyocytes, namely H9C2 (rat myoblast from embryonic heart), engineered to express a fluorescently labeled wild type RBM20 polypeptide or various fluorescently labeled RBM20 mutant polypeptides having a single point mutation in domains outside of the RS-rich domain. Specifically, H9C2 cells were engineered using transient transfection to express a wild type RBM20 polypeptide linked to a Dendra2 label or a mutant RBM20 polypeptide (E913K, R716Q, or V535L) linked to a Dendra2 label. Following transfection, cells were incubated to allow for RBM20 polypeptide expression. Fluorescent images of cells, and/or portions of cells, were captured using a DeltaVision wide-field deconvoluted system having a 60× oil objective.

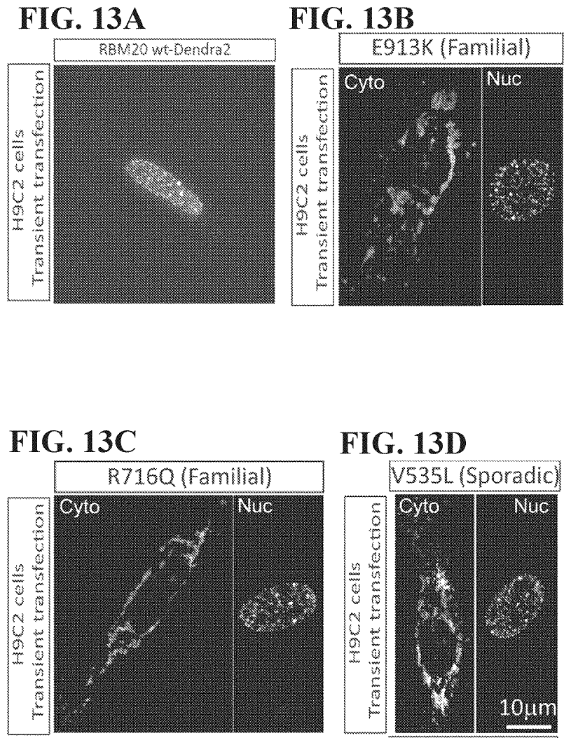

E913K (resides in the E-rich domain) and R716Q (resides between the RS-rich domain and the E-rich domain) both represent familial RBM20 polypeptide mutations. V535L mutation resides in the RPM domain, and represents a sporadic RBM20 polypeptide mutation. As shown in FIGS. 13A-13D, in H9C2 cells transfected with a wild type RBM20 polypeptide, RBM20 condensates were primarily observed in the nucleus (FIG. 13A), and in H9C2 cells transfected with the noted RBM20 mutant polypeptides there was a mixed phenotype of nuclear and cytoplasmic condensate (FIG. 13B, E913K RBM20; FIG. 13C, R716Q RBM20; FIG. 13D, V535L RBM20).

### Example 9

This example demonstrates fluorescence imaging analyses to assess for molecular components that co-localize in RBM20 condensates. Cells were engineered to express either a fluorescently labeled wild type RBM20 polypeptide or a fluorescently labeled RBM20 R636S mutant polypeptide. Following transfection, cells were incubated to allow for RBM20 polypeptide expression. Certain cells were subject to DAPI staining to visualize the nucleus. Cells were then subjected to an immunofluorescence (IF) technique for a target, such as DDX3X (a protein involved in stress granules). Fluorescent images of cells, and/or portions of cells, were captured using a DeltaVision wide-field deconvoluted system. Images were captured for both wild type and mutant RBM20 containing cells at the channel for the RBM20 label and, in the case of an IF analysis, at the channel for the labeled antibody.

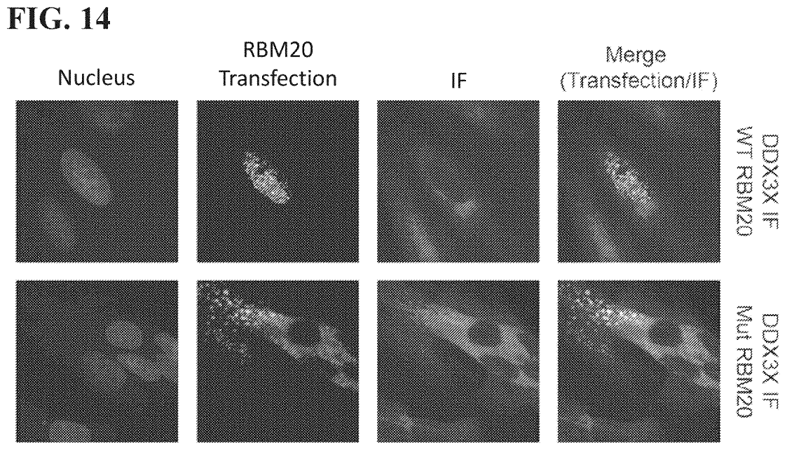

As shown in FIG. 14, DDX3X did not co-localize with RBM20 condensates in the wild type cell system, and DDX3X co-localized with cytoplasmic RBM20 condensate in the mutant RBM20 cell system. Similar experiments were conducted for IF staining of DDX5 and TDP43, which both showed similar results of co-localizing with cytoplasmic RBM20 condensate in the mutant RBM20 cell system but not in the wild type cell system; however, their partitioning into mutant RBM20 condensates was much lower compared to that of DDX3X (data not shown).

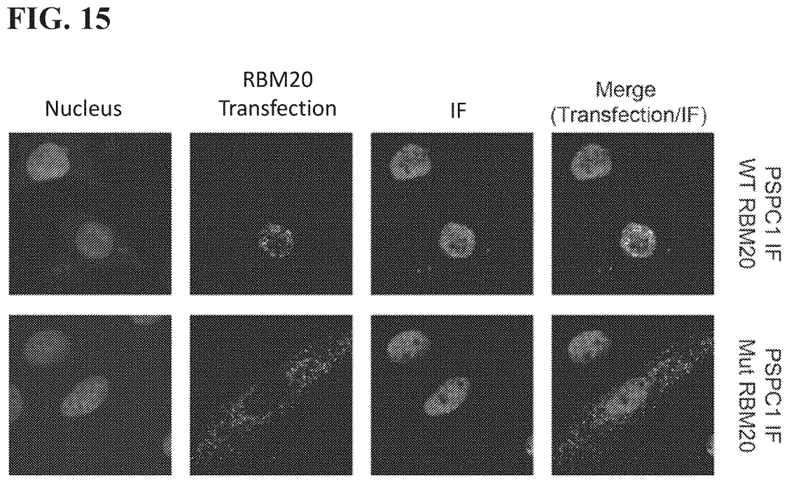

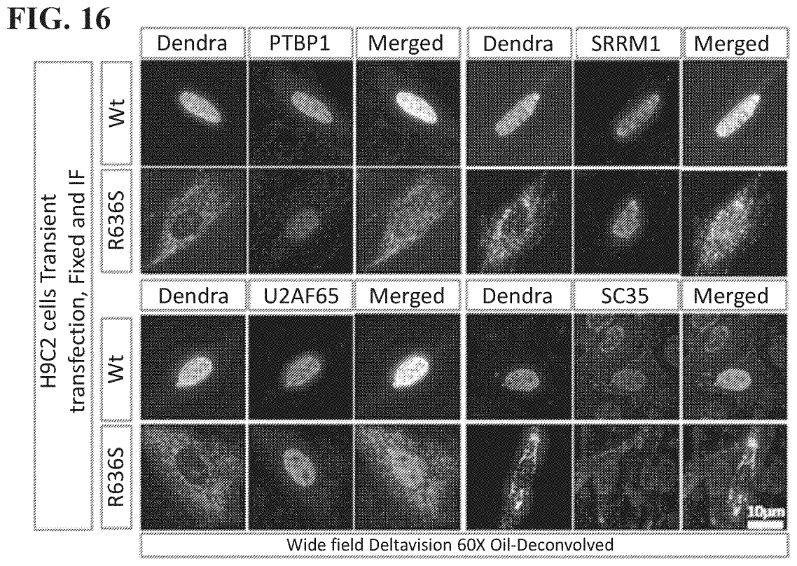

Similar experiments were also conducted for IF staining of paraspeckle proteins PSPC1, SFPQ, and NONO. As can be seen from FIG. 15, only wild type RBM20 polypeptides, but not mutant RBM20 polypeptides, partitioned into paraspeckles marked by PSPC1. Wild type RBM20 polypeptides, but not mutant RBM20 polypeptides, also partitioned similarly into paraspeckles marked by SFPQ and NONO (data not shown).

Similar experiments were also conducted for IF staining of nuclear proteins PTBP1, SRRM1, U2AF65, and SC35. As can be seen from FIG. 16, the tested nuclear proteins only co-localized with RBM20 condensates in the wild type cell system, but not with cytoplasmic RBM20 condensate in the R636S mutant RBM20 cell system.

Similar assay systems were also developed using labeled protein components other than RBM20. Specifically, a first cell system was engineered to express a mCherry-labeled wild type RBM20 polypeptide, and a GFP-labeled DDX3X. A second cell system was engineered to express a mCherry labeled R636S mutant RBM20 polypeptide, and a GFP labeled DDX3X. Following transfection, cells were incubated to allow for RBM20 polypeptide expression. Certain cells were subject to DAPI staining. Cells were then subjected to an immunofluorescence (IF) technique for an additional target, such as G3BP1 (a protein involved in stress granules). Fluorescent images of cells, and/or portions of cells, were captured using a DeltaVision wide-field deconvoluted system. Images were captured for both wild type and mutant RBM20 containing cells at the channel for the RBM20 label, at the channel for the DDX3X label, and at the channel for the labeled antibody specific for G3BP1.

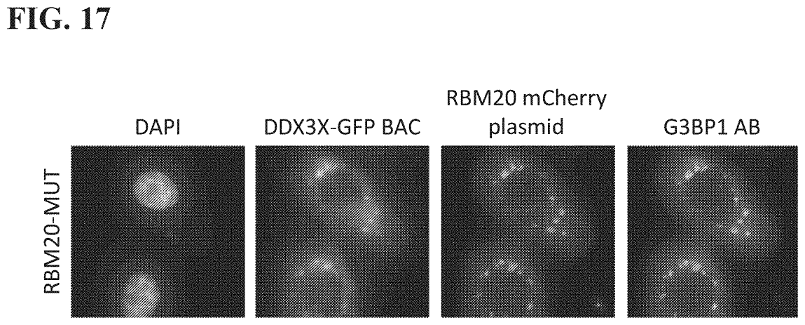

As shown in FIG. 17, DDX3X co-localized with cytoplasmic RBM20 condensate in the mutant RBM20 cell system. Additionally, stress granule marker G3BP1 co-localized with cytoplasmic RBM20 condensate in mutant RBM20 cell systems.

### Example 10

This example demonstrates cytotoxicity analyses of H9C2 cells (rat myoblast from embryonic heart) transfected with inducible wild type and R636S mutant RBM20 polypeptides.

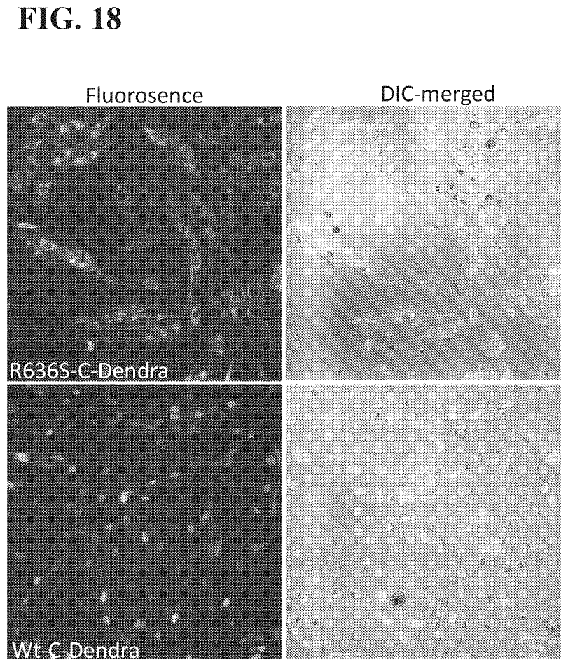

H9C2 cells were engineered to inducibly express either a wild type RBM20 polypeptide linked to a Dendra2 label, or a R636S mutant RBM20 polypeptide linked to a Dendra2 label, under a TetON control. Fluorescent and DIC images of cells, and/or portions of cells, were captured using a DeltaVision wide-field deconvoluted system. As can be seen from FIG. 18, about 24 hours post-induction, about 90% H9C2 cells induced to express wild type RBM20 polypeptide showed nuclear condensates, while those induced to express R636S mutant RBM20 polypeptides showed cytoplasmic condensates. RBM20 expression was also verified by western blot using H9C2 cell lysates (data not shown).

For cytotoxicity analyses, non-induced cells were seeded into a 96-well plate in the amount of about 1,500 cells per well. Incucyte® NucLight Rapid Red Reagent (cell permeable DNA stain) was added into each well for live-cell nuclear labeling (T0). Doxycycline was added at a final concentration of 6 μg/mL one day after adding nuclear dye, to induce protein expression, or not added as a control. Real-time quantification of live cells were conducted using the Incucyte® Live-Cell Analysis System, starting from T0, then every two hours until the end of Day 3 (T3). The nuclear dye signals over time were normalized to that of T0.

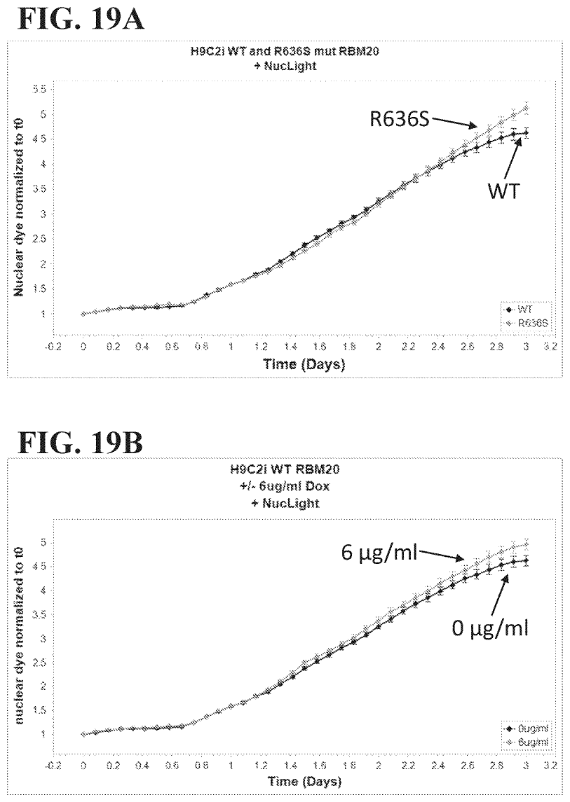

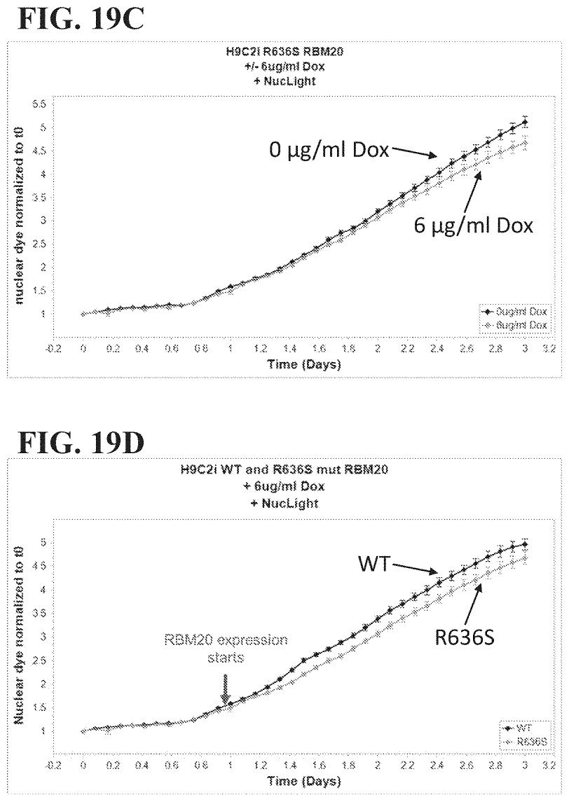

As can be seen from FIG. 19A, non-induced H9C2 cells carrying wild type or R636S mutant RBM20 plasmid did not differ much in cell growth over time. Wild type RBM20 polypeptide expression in H9C2 cells also did not affect cell proliferation, compared to non-induced cells (FIG. 19B). R636S mutant RBM20 polypeptide expression affected cell proliferation (FIG. 19C), and these H9C2 cells showed slower growth compared to H9C2 cells expressing wild type RBM20 polypeptides (FIG. 19D).

Cytotoxicity was also tested by real-time apoptosis monitoring using RealTime-Glo™ Annexin V Apoptosis and Necrosis Assay (Promega). During apoptosis, phosphatidylserine (PS) is exposed on the outer leaflet of cell membranes, which can be bound by Annexin V luciferase fusion proteins, leading to luminescence (RLU) signal.

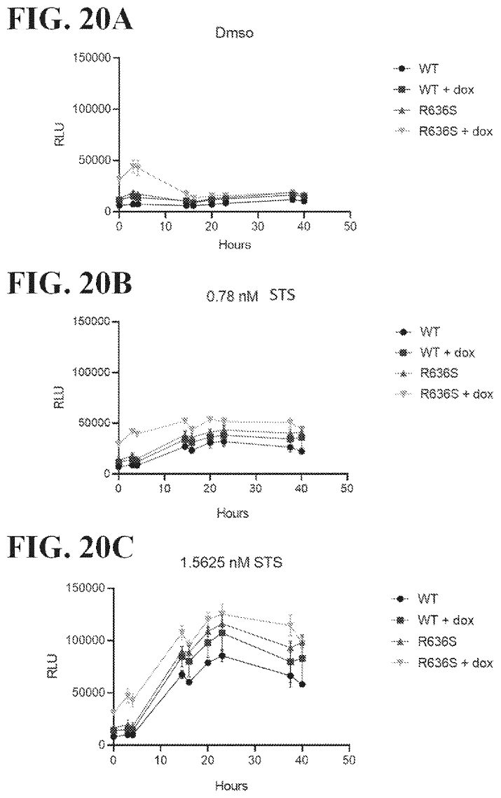

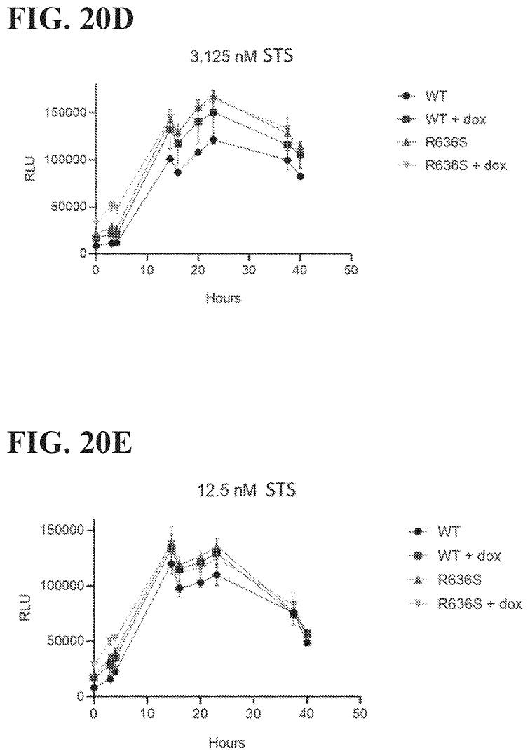

The aforementioned non-induced H29C cells were seeded into 96-well plate. Doxycycline was immediately added to induce protein expression, or not added as a control. 24-hour post doxycycline induction (or no induction), apoptosis assay reagents were added into each well, with or without apoptosis inducer Staurosporine (STS) (T0). Then luminescence was monitored for 40 hours. As can be seen from FIGS. 20A-20E, the expression of R636S mutant RBM20 polypeptides increased basal apoptosis in H9C2 cells (see FIGS. 20A-20C with low or no STS stress); while under high STS stress (see FIGS. 20D and 20E), further induction of R636S mutant RBM20 expression did not have higher apoptosis compared to cells expressing wild type RBM20.

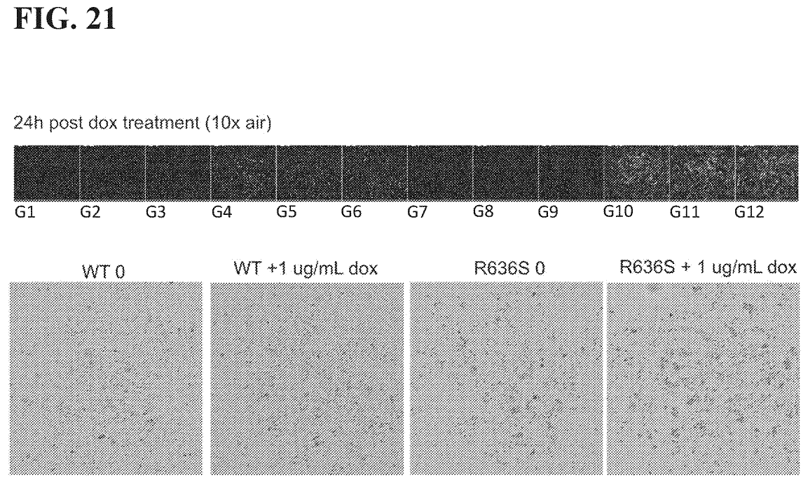

Cell apoptosis was further verified with fluorescent and DIC images of H29C cells (not treated with STS stress) captured using a DeltaVision wide-field deconvoluted system. As can be seen from FIG. 21, H29C cells induced to express wild type or R636S mutant RBM20 polypeptides both showed induced apoptosis over time; however, more floating cells (i.e., apoptotic cells) were seen in H29C cells induced to express R636S mutant RBM20 polypeptides, compared to those expressing wild type RBM20 polypeptides.

### Example 11

This example demonstrates assays for mapping components of mutant RBM20 condensates in H29C cells.

H9C2 cells were engineered to inducibly express either a wild type RBM20 polypeptide linked to a Dendra2 label (serving as control), or a R636S mutant RBM20 polypeptide linked to a Dendra2 label, under a TetON control. Cells were induced with doxycycline at a final concentration of 4 μg/mL. One day post-induction, cells were fixed and antibodies (about 200 antibodies) against test proteins (about 100 test proteins) were added for IF staining. Cells were also stained with DAPI. Fluorescent images of cells, and/or portions of cells, were captured using a DeltaVision wide-field deconvoluted system.

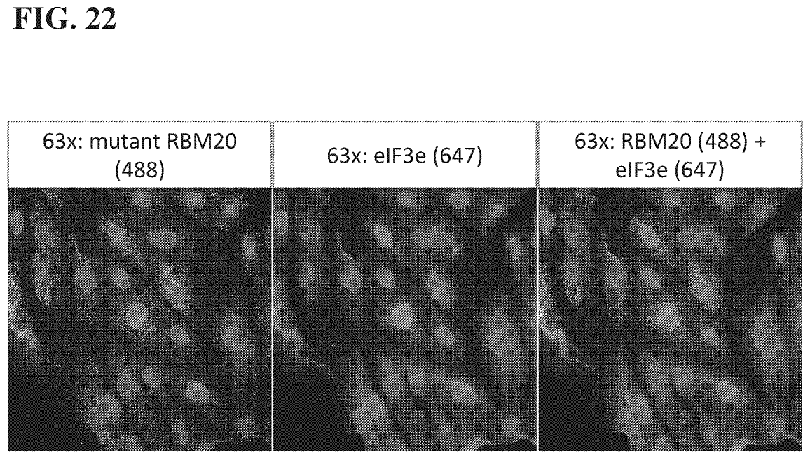

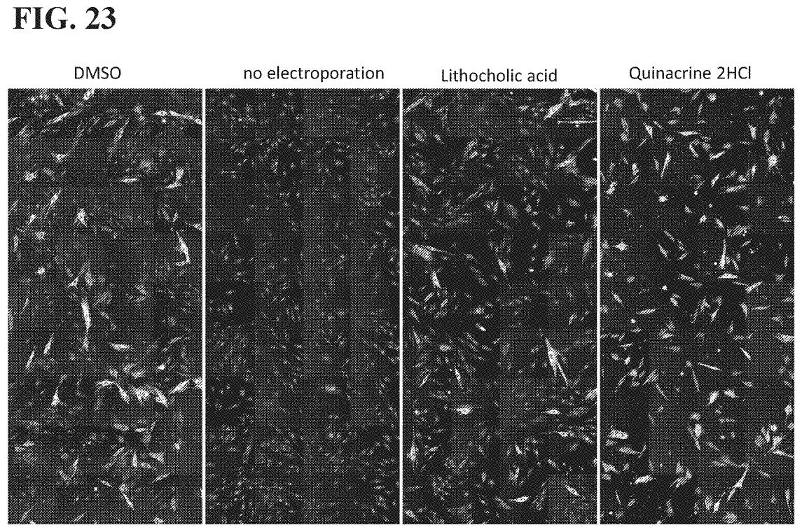

FIG. 22 shows that stress granule protein elF3e co-localized with cytoplasmic R636S mutant RBM20 condensates, suggesting that elF3e partitions into R636S mutant RBM20 condensates in the cytoplasm. Of all evaluated proteins, 30 showed partitioning into R636S mutant RBM20 condensates, many of which were stress granule proteins.

### Example 12

This example demonstrates an assay for screening a compound that modulates a characteristic associated with one or more RBM20 condensates and/or the RBM20 polypeptide. H9C2 cells were engineered using transient transfection to express a mutant R636S RBM20 polypeptide linked to a Dendra2 label. Following transfection, cells were incubated to allow for RBM20 polypeptide expression for about 16 hours. H9C2 cells were seeded into 384-well plates, and admixed with a control (DMSO) or 20 μM test compound, and then incubated for 24 hours prior to fixation, staining (e.g., with DAPI), and capturing of fluorescent images. H9C2 cells not transfected with mutant R636S RBM20 polypeptide served as controls. Three replicates were run for each compound. Fluorescent images of cells, and/or portions of cells, were captured using a DeltaVision wide-field deconvoluted system.

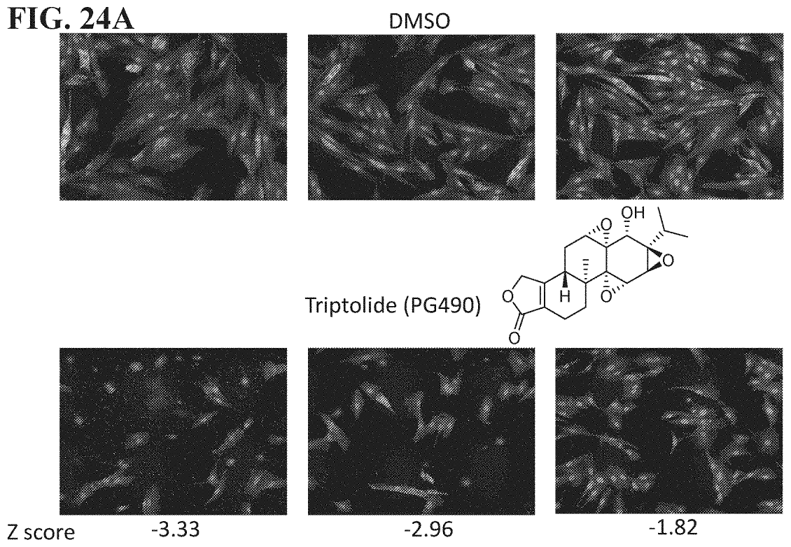

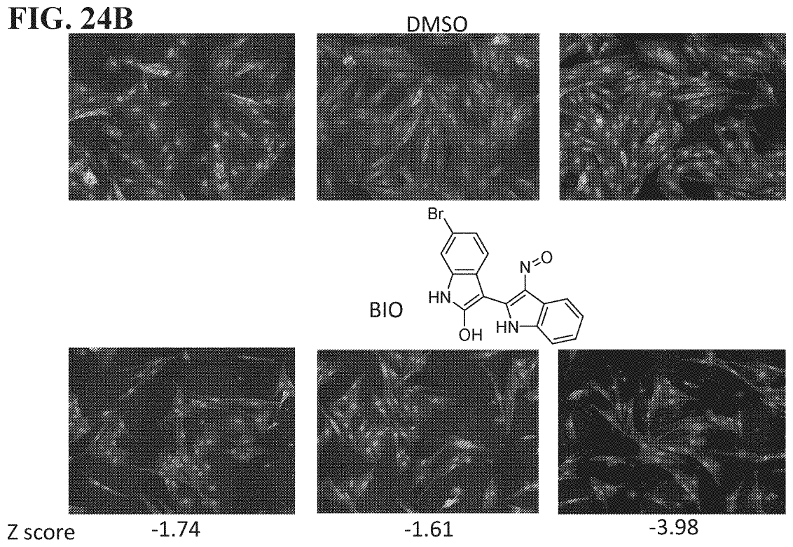

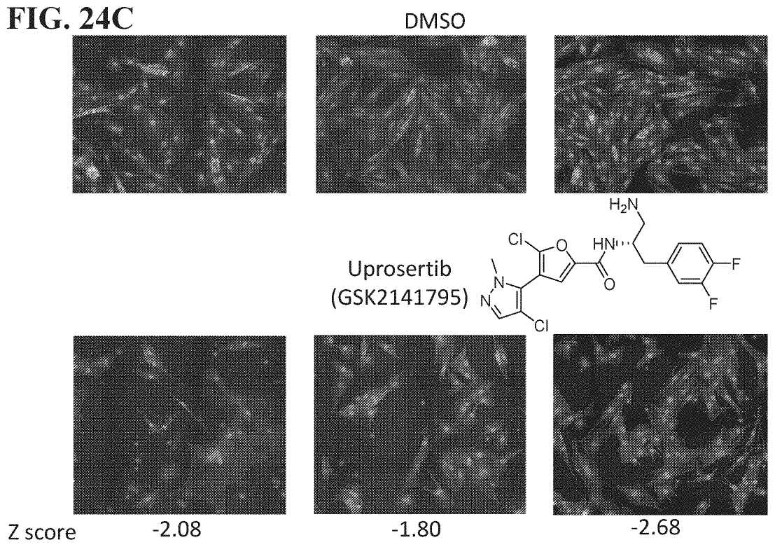

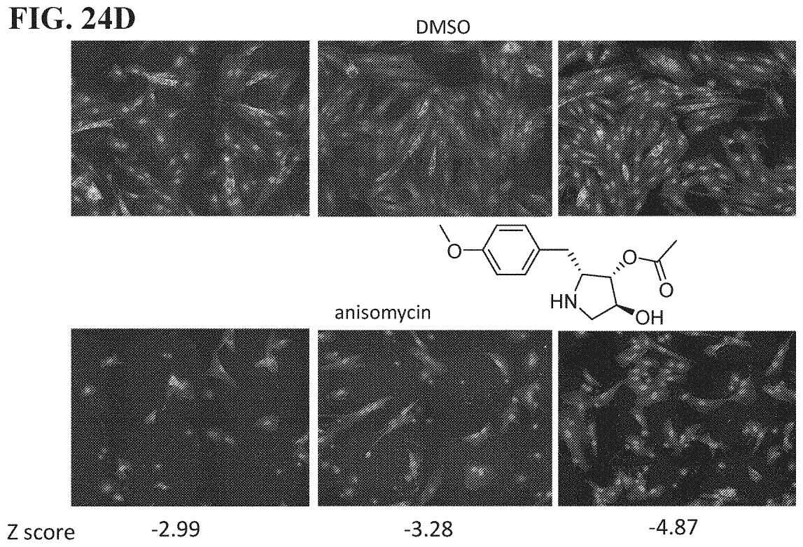

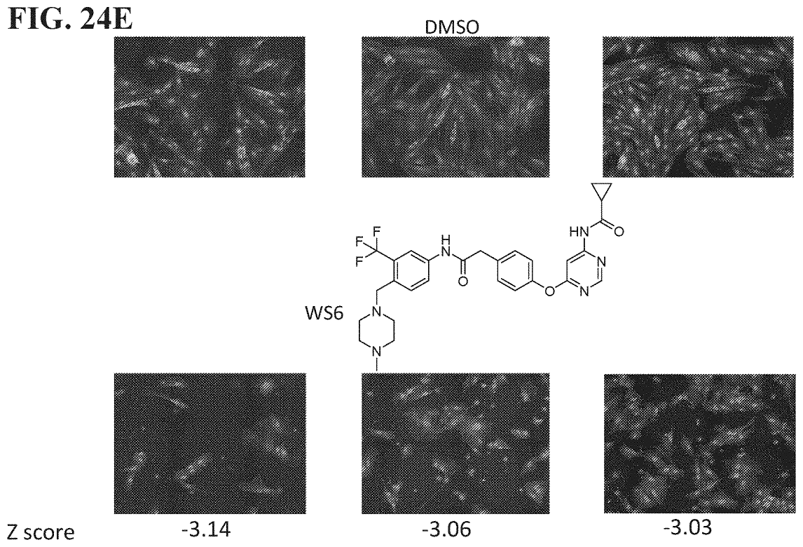

Images of the H9C2 cells treated with lithocholic acid, Quinacrine 2HCl, and a reference (DMSO) are shown in FIG. 23. Non-transfected H9C2 cells served as control. FIG. 23 shows that lithocholic acid was able to reduce cytoplasmic mutant R636S RBM20 condensates, while Quinacrine 2HCl increased cytoplasmic mutant R636S RBM20 condensates. FIGS. 24A-24E show that compounds Triptolide (PG490) (FIG. 24A), BIO (FIG. 24B), Uprosertib (GSK2141795) (FIG. 24C), anisomycin (FIG. 24D), and WS6 (FIG. 24E) were all able to reduce cytoplasmic mutant R636S RBM20 condensates. Their down-regulation levels are indicated as Z score under each panel.

What is claimed is:

1. A method of identifying a compound that modulates a characteristic associated with one or more condensates comprising a RBM20 polypeptide (“RBM20 condensates”) in the cytoplasm of a cell and/or the RBM20 polypeptide in the cytoplasm of a cell, the method comprising:
(a) admixing the compound and a composition comprising the cell,
wherein (i) the cell comprises the one or more RBM20 condensates, and/or (ii) the one or more RBM20 condensates form in the cell after the compound contacts the composition; and
(b) determining the characteristic associated with the one or more RBM20 condensates and/or the RBM20 polypeptide,
wherein a modulation in the characteristic, as compared to a reference, indicates that the compound modulates the characteristic associated with the one or more RBM20 condensates and/or the RBM20 polypeptide.

2. The method of claim 1, wherein the characteristic associated with the one or more RBM20 condensates and/or the RBM20 polypeptide is based on any one or more of the following:
(i) location of the one or more RBM20 condensates;
(ii) distribution of the one or more RBM20 condensates and/or the RBM20 polypeptide;
(iii) number of the one or more RBM20 condensates;
(iv) size of the one or more RBM20 condensates;
(v) ratio of the amount of one or more RBM20 condensates and a reference condensate;
(vi) a functional activity associated with the one or more RBM20 condensates;
(vii) composition of the one or more RBM20 condensates;
(viii) co-localization of the one or more RBM20 condensates with a biomolecule;
(ix) diffusion coefficient of a component of the one or more RBM20 condensates;
(x) stability of the one or more RBM20 condensates;
(xi) dissolution or reduction in size of the one or more RBM20 condensates;
(xii) surface area of the one or more RBM20 condensates;
(xiii) sphericity of the one or more RBM20 condensates;
(xiv) liquidity of the one or more RBM20 condensates;
(xv) solidification of the one or more RBM20 condensates;
(xvi) location of the RBM20 polypeptide;
(xvii) amount of the RBM20 polypeptide or a precursor thereof;
(xviii) condensate partitioning of the RBM20 polypeptide into the one or more RBM20 condensates;
(xix) a functional activity associated with the RBM20 polypeptide;
(xx) aggregation of the RBM20 polypeptide;
(xxi) post-translational modification status of the RBM20 polypeptide; and
(xxii) amount of a RBM20 polypeptide degradation product.

3. The method of claim 2, wherein the modulation in the characteristic is based on a decrease in the number of the one or more RBM20 condensates in the cytoplasm of the cell.

4. The method of claim 2 or 3, wherein the modulation in the characteristic is based on a decrease in the amount of the RBM20 polypeptide or a precursor thereof in the cytoplasm of the cell.

5. The method of any one of claims 2-4, wherein the modulation in the characteristic is based on dissolution or reduction in size of the one or more RBM20 condensates in the cytoplasm of the cell.

6. The method of any one of claims 2-5, wherein the modulation in the characteristic is based on a decrease in the functional activity associated with the one or more RBM20 condensates and/or the RBM20 polypeptide in the cytoplasm of the cell.

7. The method of claim 1, wherein the characteristic associated with the one or more RBM20 condensates and/or the RBM20 polypeptide comprises location of the one or more RBM20 condensates, distribution of the one or more RBM20 condensates and/or the RBM20 polypeptide, number of the one or more RBM20 condensates, size of the one or more RBM20 condensates, and ratio of the amount of one or more RBM20 condensates and a reference condensate.

8. The method of claim 1, wherein the characteristic associated with the one or more RBM20 condensates and/or the RBM20 polypeptide comprises composition of the one or more RBM20 condensates, and co-localization of the one or more RBM20 condensates with a biomolecule.

9. The method of claim 7 or 8, wherein the characteristic associated with the one or more RBM20 condensates and/or the RBM20 polypeptide further comprises the functional activity associated with the one or more RBM20 condensates.

10. The method of claim 1, wherein the characteristic associated with the one or more RBM20 condensates and/or the RBM20 polypeptide comprises stability of the one or more RBM20 condensates, dissolution or reduction in size of the one or more RBM20 condensates, and surface area of the one or more RBM20 condensates.

11. The method of claim 1, wherein the characteristic associated with the one or more RBM20 condensates and/or the RBM20 polypeptide comprises sphericity of the one or more RBM20 condensates, liquidity of the one or more RBM20 condensates, and solidification of the one or more RBM20 condensates.

12. The method of claim 1, wherein the characteristic associated with the one or more RBM20 condensates and/or the RBM20 polypeptide comprises location of the RBM20 polypeptide, and amount of the RBM20 polypeptide or a precursor thereof.

13. The method claim 12, wherein the characteristic associated with the one or more RBM20 condensate and/or the RBM20 polypeptide further comprises post-translational modification status of the RBM20 polypeptide.

14. The method of claim 12 or 13, wherein the characteristic associated with the one or more RBM20 condensate and/or the RBM20 polypeptide further comprises the functional activity associated with the RBM20 polypeptide.

15. The method of claim 1, wherein the characteristic associated with the one or more RBM20 condensates and/or the RBM20 polypeptide comprises co-localization of the one or more RBM20 condensates with a biomolecule, and diffusion coefficient of a component of the one or more RBM20 condensates.

16. The method of claim 1, wherein the characteristic associated with the one or more RBM20 condensates and/or the RBM20 polypeptide comprises stability of the one or more RBM20 condensates, and dissolution or reduction in size of the one or more RBM20 condensates.

17. The method of claim 1, wherein the characteristic associated with the one or more RBM20 condensates and/or the RBM20 polypeptide comprises surface area of the one or more RBM20 condensates, sphericity of the one or more RBM20 condensates, liquidity of the one or more RBM20 condensates, and solidification of the one or more RBM20 condensates.

18. The method of any one of claims 1-17, wherein the RBM20 polypeptide is a wild type RBM20 polypeptide.

19. The method of any one of claims 1-17, wherein the RBM20 polypeptide is a mutant RBM20 polypeptide.

20. The method of claim 19, wherein the mutant RBM20 polypeptide comprises a mutation in an intrinsically disorder region (IDR).

21. The method of claim 19 or 20, wherein the mutant RBM20 polypeptide comprises a mutation in an RS-rich region.

22. The method of any one of claims 19-21, wherein the mutant RBM20 polypeptide comprises a mutation in one or more of the following positions: arginine 634, serine 635, arginine 636, serine 637, and proline 638.

23. The method of claim 22, wherein the mutant RBM20 polypeptide comprises one or more of the following mutations: R636S, R636C, R636H, R634Q, S637G, P638L, S635A, S635E, and S637E.

24. The method of claim 19, wherein the mutant RBM20 polypeptide comprises one or more of the following mutations: E913K, R716Q, and V535L.

25. The method of any one of claims 1-24, wherein the RBM20 polypeptide is heterologously expressed in the cell.

26. The method of any one of claims 1-24, wherein the RBM20 polypeptide is homologously expressed in the cell.

27. The method of any one of claims 1-26, wherein the cell is a model of a cardiac cell type.

28. The method of any one of claims 1-27, wherein the cell is a cardiomyocyte.

29. The method of claim 27 or 28, wherein the cell is a Rat H9C2 cell.

30. The method of claim 27 or 28, wherein the cell is a human AC-16 cell, a patient-derived cardiomyocyte, a human induced pluripotent stem cell differentiated to a cardiomyocyte, or a stem cell differentiated to a cardiomyocyte.

31. The method of any one of claims 1-26, wherein the cell is selected from the group consisting of: a HeLa cell, a U2OS cell, a human embryonic kidney cell, a human induced pluripotent stem cell, and a stem cell.

32. The method of any one of claims 1-31, wherein the cell is homozygous for alleles encoding the RBM20 polypeptide.

33. The method of any one of claims 1-31, wherein the cell is heterozygous for alleles encoding the RBM20 polypeptide.

34. The method of any one of claims 1-33, wherein the reference comprises an aliquot of the composition comprising the cell admixed with a control agent.

35. The method of any one of claims 1-34, further comprising imaging at least a portion of the composition or the cell.

36. The method of any one of claims 1-35, wherein the method further comprises determining one or more cellular features of the cell.

37. The method of any one of claims 1-36, further comprising contacting at least a portion of the composition or the cell with a fixative.

38. The method of any one of claims 1-37, further comprising contacting at least a portion of the composition or the cell with a stain.

39. The method of any one of claims 1-38, further comprising assessing the identified compound using a second cell-based assay.

40. The method of any one of claim 1-39, further comprising assessing the identified compound using an in vitro assay.

41. A method of identifying a compound that reduces the size and/or number of condensates comprising a RBM20 polypeptide (“RBM20 condensates”) in the cytoplasm of a cell, the method comprising:
(a) determining the size and/or number of the RBM20 condensates in at least a portion of the cytoplasm of the cell subjected to the compound; and
(b) comparing the size and/or number of the RBM20 condensates with a reference, thereby identifying the compound that reduces the size and/or number of the RBM20 condensates in the cytoplasm of the cell.

42. The method of claim 41, wherein the compound reduces the number of RBM20 condensates.

43. The method of claim 41 or 42, wherein the compound reduces the size of RBM20 condensates.

44. A method of identifying a compound that prevents formation or growth of one or more condensates comprising a RBM20 polypeptide (“RBM20 condensates”) in the cytoplasm of a cell, the method comprising:
(a) combining the compound and a composition comprising the cell,
wherein (i) the cell comprises the one or more RBM20 condensates, and/or (ii) the one or more RBM20 condensates form after the compound contacts the composition; and
(b) obtaining a first measurement of the size and/or number of the one or more RBM20 condensates in at least a portion of the cytoplasm of the cell; and
(c) comparing the first measurement to a reference, thereby identifying a compound that prevents formation or growth of the one or more RBM20 condensates in the cytoplasm of the cell.

45. The method of claim 44, wherein the reference comprises an aliquot of the composition comprising the cell admixed with a control agent.

46. The method of claim 44, wherein the reference is a second measurement of the size and/or number of the one or more RBM20 condensates in at least a portion of the cytoplasm of the cell, and wherein the second measurement is taken at a different time than the first measurement.

47. The method of any one of claims 44-46, further comprising subjecting the cell to a condition that promotes formation of the one or more RBM20 condensates.

48. A method of identifying a compound that decreases the amount of a RBM20 polypeptide in the cytoplasm of a cell, the method comprising:
(a) combining the compound and a composition comprising the cell; and
(b) obtaining a first measurement of the amount of the RBM20 polypeptide in at least a portion of the cytoplasm of the cell; and
(c) comparing the first measurement to a reference, thereby identifying a compound that decreases the amount of the RBM20 polypeptide in the cytoplasm of the cell.

49. The method of claim 48, wherein the reference comprises an aliquot of the composition comprising the cell admixed with a control agent.

50. The method of claim 49, wherein the reference is a second measurement of the amount of the RBM20 polypeptide in at least a portion of the cytoplasm of the cell, and wherein the second measurement is taken at a different time than the first measurement.

51. A method of identifying a compound that modulates a characteristic associated with one or more condensates comprising a RBM20 polypeptide (“RBM20 condensates”), the method comprising:
(a) admixing the compound and a solution comprising the one or more RBM20 condensates and an extra-condensate solution; and
(b) determining the characteristic associated with the one or more RBM20 condensates,
wherein a modulation in the characteristic, as compared to a reference, indicates that the compound modulates the characteristic associated with the one or more RBM20 condensates.

52. A method of identifying a compound that modulates a characteristic associated with one or more condensates comprising a RBM20 polypeptide (“RBM20 condensates”) and/or the RBM20 polypeptide, the method comprising:
(a) combining an agent and a solution comprising the RBM20 polypeptide in the presence of the compound,
wherein the agent is capable of causing the formation of the one or more RBM20 condensates and the one or more RBM20 condensates form after the agent contacts the solution; and
(b) determining the characteristic associated with the one or more RBM20 condensates and/or the RBM20 polypeptide,
wherein a modulation in the characteristic, as compared to a reference, indicates that the compound modulates the characteristic associated with the one or more RBM20 condensates and/or the RBM20 polypeptide.

53. The method of claim 51 or 52, wherein the characteristic associated with the one or more RBM20 condensates and/or the RBM20 polypeptide is based on any one or more of the following:
(i) number of the one or more RBM20 condensates;
(ii) composition of the one or more RBM20 condensates;
(iii) size of the one or more RBM20 condensates;
(iv) stability of the one or more RBM20 condensates;
(v) dissolution or reduction in size of the one or more RBM20 condensates;
(vi) surface area of the one or more RBM20 condensates;
(vii) sphericity of the one or more RBM20 condensates;
(viii) liquidity of the one or more RBM20 condensates;
(ix) solidification of the one or more RBM20 condensates;
(x) amount of the RBM20 polypeptide not in the one or more RBM20 condensates;
(xi) partitioning of the RBM20 polypeptide into the one or more RBM20 condensates; and
(xii) aggregation of the RBM20 polypeptide.

54. A method of identifying a compound that modulates the partitioning of a biomolecule for a condensate comprising a RBM20 polypeptide (“RBM20 condensate”), the method comprising:
(a) admixing the compound and a composition comprising a cell,
wherein (i) the cell comprises the RBM20 condensate, and/or (ii) the RBM20 condensate forms in the cell after the compound contacts the composition; and
(b) determining the partitioning of the biomolecule for the RBM20 condensates.

55. The method of claim 54, wherein the biomolecule is a non-RBM20 polypeptide.

56. The method of claim 54, wherein the biomolecule is a wild type RBM20 polypeptide.

57. The method of any one of claims 54-56, wherein the RBM20 condensate comprising the RBM20 polypeptide comprises a mutant RBM20 polypeptide.

58. A method of identifying a compound useful for treating a RBM20-associated disease, the method comprising identifying a compound according to any one of the methods of claims 1-57.

59. The method of claim 58, wherein the RBM20-associated disease is a cardiomyopathy.

60. The method of claim 59, wherein the cardiomyopathy is dilated cardiomyopathy.


SEQUENCE LISTING (3 sequences)

SEQ ID NO: 1 - protein, 1227 aa, Homo sapiens
MVLAAAMSQDADPSGPEQPDRVACSVPGARASPAPSGPRGMQQPPPPPQPPPPPQAGLPQIIQNAAKLLDKNPFSVSNPNPLLPSPASLQLAQLQAQLTLHRLKLAQTAVTNNTAAATVLNQVLSKVAMSQPLFNQLRHPSVITGPHGHAGVPQHAAAIPSTRFPSNAIAFSPPSQTRGPGPSMNLPNQPPSAMVMHPFTGVMPQTPGQPAVILGIGKTGPAPATAGFYEYGKASSGQTYGPETDGQPGFLPSSASTSGSVTYEGHYSHTGQDGQAAFSKDFYGPNSQGSHVASGFPAEQAGGLKSEVGPLLQGTNSQWESPHGFSGQSKPDLTAGPMWPPPHNQPYELYDPEEPTSDRTPPSFGGRLNNSKQGFIGAGRRAKEDQALLSVRPLQAHELNDFHGVAPLHLPHICSICDKKVFDLKDWELHVKGKLHAQKCLVFSENAGIRCILGSAEGTLCASPNSTAVYNPAGNEDYASNLGTSYVPIPARSFTQSSPTFPLASVGTTFAQRKGAGRVVHICNLPEGSCTENDVINLGLPFGKVTNYILMKSTNQAFLEMAYTEAAQAMVQYYQEKSAVINGEKLLIRMSKRYKELQLKKPGKAVAAIIQDIHSQRERDMFREADRYGPERPRSRSPVSRSLSPRSHTPSFTSCSSSHSPPGPSRADWGNGRDSWEHSPYARREEERDPAPWRDNGDDKRDRMDPWAHDRKHHPRQLDKAELDERPEGGRPHREKYPRSGSPNLPHSVSSYKSREDGYYRKEPKAKWDKYLKQQQDAPGRSRRKDEARLRESRHPHPDDSGKEDGLGPKVTRAPEGAKAKQNEKNKTKRTDRDQEGADDRKENTMAENEAGKEEQEGMEESPQSVGRQEKEAEFSDPENTRTKKEQDWESESEAEGESWYPTNMEELVTVDEVGEEEDFIVEPDIPELEEIVPIDQKDKICPETCLCVTTTLDLDLAQDFPKEGVKAVGNGAAEISLKSPRELPSASTSCPSDMDVEMPGLNLDAERKPAESETGLSLEDSDCYEKEAKGVESSDVHPAPTVQQMSSPKPAEERARQPSPFVDDCKTRGTPEDGACEGSPLEEKASPPIETDLQNQACQEVLTPENSRYVEMKSLEVRSPEYTEVELKQPLSLPSWEPEDVFSELSIPLGVEFVVPRTGFYCKLCGLFYTSEETAKMSHCRSAVHYRNLQKYLSQLAEEGLKETEGADSPRPEDSGIVPRFERKKL

SEQ ID NO: 2 - DNA, 27 bp, Artificial Sequence
  Synthetic Construct
ctgccccccctggagcgcctgaccctg

SEQ ID NO: 3 - protein, 9 aa, Artificial Sequence
  Synthetic Construct
LPPLERLTL